SEQUENCE LISTING (3 sequences)

SEQ ID NO: 1 - protein, 1035 aa, synthetic construct
MPEQERQITAREGASRKILSKLSLPTRAWEPAMKKSFAFDNVGYEGGLDGLGPSSQPQKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 2 - protein, 1325 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDQPDLAPGLTTIGASPTQTVTLVTQPVVTKETAISKLEMPSSLMLEVPTHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMET

SEQ ID NO: 3 - protein, 497 aa, synthetic construct
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

(19) United States
(12) Patent Application Publication
(10) Pub. No.: US 2024/0287470 A1
(43) Pub. Date: Aug. 29, 2024

(54) CETYLTRIMETHYLAMMONIUM BROMIDE (CTAB) AS A LYSIS AND FLOCCULATION REAGENT IN GENE THERAPY DOWNSTREAM PROCESSES

(71) Applicant: Pfizer Inc., New York, NY (US)

(72) Inventors: Hannah Kim BARE, Manchester, MO (US); Alexander BERRILL, Wildwood, MO (US); Yulia M IVANOVA, Ballwin, MO (US)

(21) Appl. No.: 18/586,896

(22) Filed: Feb. 26, 2024

Related U.S. Application Data
(60) Provisional application No. 63/487,300, filed on Feb. 28, 2023.

(51) Int. Cl. C12N 7/00; C07K 14/005; C12N 5/07; C12N 5/071

(52) U.S. Cl. CPC C12N 7/00; C07K 14/005; C12N 5/0601; C12N 5/0602; C12N 2750/14122; C12N 2750/14151

(57) ABSTRACT

The present disclosure describes improved methods for use in purifying biological products made by host cells. In some embodiments, the improved methods comprise one or more steps of lysing host cells to release the biological product and precipitating host cell DNA, using a detergent such as cetyltrimethylammonium bromide (CTAB). In some embodiments, the biological product is a vaccine, or a viral vector for gene therapy, such as an AAV vector or a lentiviral vector.

### REFERENCE TO SEQUENCE LISTING

This application is being filed electronically via EFS-Web and includes an electronically submitted sequence listing in .xml format. The .xml file contains a sequence listing entitled “PC072863A Sequence Listing.xml” created on Feb. 1, 2024 and having a size of 6.35 KB. The sequence listing contained in this .xml file is part of the specification and is herein incorporated by reference in its entirety.

### BACKGROUND

Various types of cultured cells may be used to make therapeutically desirable molecules. Such molecules include naturally occurring compounds made by unmodified cells, however cells may also be modified using genetic engineering technology to produce simple and more complex molecules and even supramolecular structures like viruses. For example, bacteria may be transfected with plasmids to express relatively simple proteins, such as human growth hormone or insulin. Eukaryotic cells, such as yeast, mammalian, or insect cells, may be transfected with foreign genetic material to enable the cells to produce more complicated proteins, such as enzymes, clotting factors, antibodies and recombinant adeno-associated viral (ray) vectors. This technology has enabled the efficient production of biological products with important medical and industrial applications that would not otherwise be possible.

Lysis of cells that make therapeutically desirable molecules is one of the first steps in the process of purifying a molecule from cell culture. Cell lysis is a process whereby the outer boundary or the cell membrane is damaged or removed to allow release of inter-cellular materials, such as DNA, RNA, proteins or organelles. Cell lysis may occur either by chemical lysis (e.g. pH, detergent, high osmolality solution) or physical lysis (e.g. multiple freeze-thaw cycles, microfluidization, sonication). While physical lysis processes are common and very effective, these techniques require specialized equipment and can be challenging to scale as compared to chemical lysis processes.

In the gene therapy field, work continues in order to understand the extra- and intracellular distribution of AAV capsids and whether or not this depends on capsid serotype and cell culture conditions. However, host cell lysis is still required to ensure complete release of AAV capsids from the cells and for their subsequent recovery (Vandenberghe et al., Human Gene Therapy, 2010, 21:1251-1257). Octoxynol-9 (also known as Triton X-100) is a common cell lysis detergent and has been shown to be effective in the release of rAAV vectors from cells. While cell lysis using octoxynol-9 is relatively inexpensive, use of the detergent is concentration and time dependent, with optimal concentrations ranging from 0.1% to 0.5% w/v (Dias Florencio et al., Methods and Clinical Development, 2015, 2:15024). Also, concerns about environmental impacts of octoxynol-9 degradation products have been raised and it is expected that inclusion of the chemical on the Registration, Evaluation, Authorization and Restriction of Chemicals (REACH) list of banned substances may result in limitation of its use by the European Union (EU). Alternative chemical lysis agents such as polysorbate 20 (PS20) (also known as Tween 20) have been shown to release comparable levels of rAAV vectors as compared to octoxynol-9, however PS20 may lower the efficacy of chromatographic processes during purification (Application note, KA875220218AN, Cytiva). Acidic buffers, such as citrate, pH 4.2, may be used for cell lysis without negatively impacting downstream chromatography and a low pH approach was shown to generate clarified lysates with lower contaminant levels (Kimura et. al., Scientific Reports, 2019, 9:13601).

After the cellular lysis step, removal, or degradation of released nucleic acids is required prior to proceeding to subsequent purification steps. By degrading nucleic acid into smaller fragments, the resulting solution is less viscous which improves filterability. In addition, degradation decreases the risk of carrying longer DNA fragments through the purification process and into the final product. A common approach for DNA degradation is the use of a DNAse enzyme such as Benzonase® (MilliporeSigma). A disadvantage of Benzonase is its low tolerance to high salt concentrations that are often needed for optimal capsid recovery, as well as its relatively high cost.

An alternative method for removing cellular contaminants from a cell lysate is treatment with a detergent which causes flocculation of the contaminants. A commonly used flocculant is domiphen bromide (DB), a positively charged quaternary ammonium compound that binds negatively charged DNA. The hydrophobic tail of DB is thought to drive precipitation of DNA-DB complexes, most likely via formation of micelles. Its use as a DNA flocculation agent in adenovirus and adeno-associated viral purification trains is well documented (Goerke et al., Wiley InterScience 2004). During flocculation, host cell DNA (HCDNA) and host cell protein (HCP) precipitate and settle out of solution as a flocculant mass. The supernatant, which contains the molecules of interest (e.g., rAAV vectors), is more efficiently filtered to produce a clarified lysate solution following the flocculation. Depletion of HCDNA and HCP from a clarified lysate often leads to better performance across chromatography steps and longer column lifetimes.

Various methods for the production of a clarified lysate have been developed and conditions selected may be driven by the feasibility of purification at a manufacturing site, nature of a target molecule, cost, safety and quality requirements for the final product. For example, not all rAAV vectors are readily purified from cells by lysis and flocculation, or at the same level of recovery (e.g., total vg recovered). Some AAV capsids (e.g., AAV3B) possess positively charged surface residues (for example arginine and lysine) and have a high affinity for negatively charged heparan sulfate proteoglycan (HSPG) receptors on the surface of cells and cell debris. (Zhang et al., Biochemistry, 2013, 52(6):6275-6285; Lerch and Chapman, Virology, 2012, 423(1):6-13). This binding of capsids to cell material reduces recovery of total vector genomes through the drug substance purification process. Accordingly, there is a need in the art for the development of purification methods for rAAV vectors (e.g., AAV2, AAV3 (including AAV3A and AAV3B), AAV6, AAV13, AAV-DJ) with surface charges that increase binding to lysed cell material and thus decrease recovery of rAAV vector genomes.

### SUMMARY OF THE INVENTION

The present disclosure addresses a need in the art by providing novel methods, compositions and systems for the preparation of a cell lysate comprising rAAV vectors, and in particular rAAV vectors with an increased affinity for negatively charged cell surface moieties (e.g., HSPG receptors). According to certain non-limiting embodiments, such rAAV vectors include, for example, those with an AAV2, AAV3 (including AAV3A and AAV3B), AAV6, AAV13 or AAV-DJ capsid serotype. However, the methods disclosed herein are also suitable for purification of rAAV vectors comprising other AAV capsid serotypes, such as AAV9. The improved methods for production of a cell lysate disclosed herein also advantageously use a single reagent (cetyltrimethylammonium bromide (CTAB)) for both lysis and flocculation (as opposed to two reagents, for example, DB and octoxynol-9), use a lower quantity of reagent as compared to other methods (e.g., 0.05% CTAB as compared to 0.5% octoxynol-9) and do not require settling of the flocculant mass which allows for faster processing and reduces loss of lysate and vector in the liquid trapped within the mass.

In some aspects, the disclosure provides a method of preparing a cell lysate, the method comprising lysing host cells and precipitating DNA released from the host cells to produce a lysate comprising a flocculant and a supernatant. In some embodiments, the host cells are suspended in a physiologically compatible fluid, forming a cell suspension, and are lysed by adding to the cell suspension a solution comprising a detergent in a concentration sufficient to cause cell lysis. In some embodiments, the concentration of the detergent is sufficient to precipitate the host cell DNA from the lysate. In some embodiments, the detergent is a long chain quaternary ammonium salt. In some embodiments, the long chain quaternary ammonium salt is cetyltrimethylammonium bromide (CTAB).

In some embodiments, the method comprises adding a solution comprising a modifier to the fluid. In some embodiments, the solution comprising the modifier is added to the fluid before the solution comprising the detergent is added to the fluid. In some embodiments, the modifier is a salt selected from the group consisting of ZnSO₄, MgSO₄, MgCl₂, Na₂SO₄, NaCl, sodium citrate, sodium acetate, ammonium acetate and a combination thereof. In some embodiments, the modifier is a salt and the salt is MgSO₄.

In some embodiments, the method further comprises mixing the cell suspension with the solution comprising the detergent, the solution comprising the modifier or both. In some embodiments, the mixing occurs for at least 5 minutes. In some embodiments, the cell suspension is mixed with the solution comprising the modifier for at least 5 minutes followed by addition of the solution comprising the detergent followed by mixing for at least 5 minutes. In some embodiments, prior to being suspended in a physiologically compatible fluid, the host cells are grown or maintained as an adherent cell culture on a substrate, or in suspension cell culture. In some embodiments, prior to lysis, the viable cell density of the host cells suspended in the physiologically compatible fluid is at least about 10×10⁶ viable cells (vc)/mL. In some embodiments, the viable cell density of the host cells suspended in the physiologically compatible fluid ranges from about 10×10⁶ to 30×10⁶ vc/mL, or from about 15×10⁶ to 25×10⁶ vc/mL. In some embodiments, the host cells are mammalian cells or insect cells. In some embodiments, the host cells are selected from the group of cells consisting of HEK293 cells, CHO cells, HeLa cells, Sf9 cells, and Sf1 cells.

In some embodiments, the final concentration of detergent in the lysate is at least 0.01% (w/v). In some embodiments, the final concentration of detergent in the lysate ranges from about 0.01% to 0.1% (w/v), from about 0.025% to 0.075% (w/v) or from about 0.04% to 0.06% (w/v). In some embodiments, the final concentration of detergent in the lysate is about 0.05% (w/v). In some embodiments, prior to lysis, the viable cell density of the host cells in the physiologically compatible fluid is at least about 10×10⁶ vc/mL and the final concentration of CTAB in the lysate is at least about 0.05% (w/v).

In some embodiments, the final concentration of modifier in the lysate is at least 10 mM. In some embodiments, the final concentration of modifier in the lysate ranges from about 10 mM to 90 mM, from about 25 mM to 75 mM, or from about 40 mM to 60 mM. In some embodiments, the final concentration of modifier in the lysate is about 50 mM. In some embodiments, prior to lysis, the viable cell density of the host cells in the physiologically compatible fluid is at least about 10×10⁶ vc/mL and the final concentration of MgSO₄ in the lysate is at least about 50 mM.

In some embodiments, the viable cell density of the host cells in the physiologically compatible fluid ranges from about 10×10⁶ vc/mL to 30×10⁶ vc/mL, the detergent is CTAB, the final concentration of CTAB in the lysate ranges from about 0.01% to 0.1% (w/v), from about 0.025% to 0.075% (w/v) or from about 0.04% to 0.06% (w/v); and the final concentration of MgSO₄ in the lysate ranges from about 10 mM to 90 mM, from about 25 mM to 75 mM or from about 40 mM to 60 mM.

In some embodiments, the viable cell density of the host cells in the physiologically compatible fluid ranges from about 15×10⁶ vc/mL to 25×10⁶ vc/mL, the detergent is CTAB, the final concentration of CTAB in the lysate ranges from about 0.01% to 0.1% (w/v), from about 0.025% to 0.075% (w/v), or from about 0.04% to 0.06% (w/v); and the final concentration of MgSO₄ in the lysate ranges from about 10 mM to 90 mM, from about 25 mM to 75 mM, or from about 40 mM to 60 mM. In some embodiments, the final concentration of CTAB is about 0.05% (w/v), and the final concentration of MgSO₄ is about 50 mM.

In some embodiments, the supernatant is separated from the flocculant by filtration.

In some embodiments, prior to lysis, the viable cell density of the host cells in the physiologically compatible fluid is at least about 10×10⁶ vc/mL, the final concentration of CTAB in the lysate is at least about 0.01% (w/v) and the final concentration of MgSO₄ in the lysate is at least about 10 mM. In some embodiments, prior to lysis, the viable cell density of the host cells in the physiologically compatible fluid is at least about 10×10⁶ vc/mL, the final concentration of CTAB in the lysate is about 0.05% (w/v) and the final concentration of the MgSO₄ in the supernatant is about 50 mM.

In some embodiments, the final concentration of CTAB in the lysate relative to the viable cell density prior to lysis is not less than 0.001% per 1×10⁶ vc/mL. In some embodiments, the viable cell density of the host cells in the physiologically compatible fluid ranges from about 10×10⁶ vc/mL to 30×10⁶ vc/mL, the final concentration of CTAB in the lysate is at least about 0.01% (w/v), at least about 0.025% (w/v) or ranges from about 0.01% to 0.1% (w/v) and the final concentration of MgSO₄ in the lysate is at least about 10 mM, at least about 25 mM, or ranges from about 25 mM to about 75 mM.

In some embodiments, the viable cell density of the host cells in the physiologically compatible fluid ranges from about 15×10⁶ vc/mL to 25×10⁶, the final concentration of CTAB in the lysate is at least about 0.01% (w/v), at least about 0.025% (w/v) or ranges from about 0.01% to 0.1% (w/v) and the final concentration of MgSO₄ in the lysate is at least about 10 mM, at least about 25 mM, or ranges from about 25 mM to about 75 mM. In some embodiments, the final concentration of CTAB is about 0.05% (w/v), and the final concentration of MgSO₄ is about 50 mM.

In some embodiments, the method further comprises filtering the supernatant and flocculant to produce a clarified lysate. In some embodiments, the clarified lysate comprises about 1.5 fold to 10 fold more, 1.5 fold to 7 fold more, or 1.5 fold to 5 fold more vector genomes (vg) as compared to a clarified lysate prepared using octoxynol-9 and domiphen bromide to lyse the host cells and precipitate the host cell DNA. In some embodiments, the clarified lysate comprises about or at least 1×10¹³ vg/liter host cell culture (vg/L), or a range from about 1×10¹³ to 5×10¹⁴ vg/L. In some embodiments, the clarified lysate comprises about or at least 1.0×10² ng host cell DNA (HCD)/1×10⁹ vector genomes (vgs), or a range from about 1.0×10² ng HCD/1×10⁹ vg to about 5.0×10⁴ ng HCD/1×10⁹ vg.

In some embodiments, the method further comprises purifying a biological product from the clarified lysate by performing a downstream purification processing step. In some embodiments, the biological product is a recombinant viral vector for expressing a heterologous gene. In some embodiments, the recombinant viral vector is an adenovirus vector, adeno-associated virus (AAV) vector, retrovirus vector, or lentivirus vector. In some embodiments, the recombinant viral vector is an AAV vector. In some embodiments, the AAV vector comprises a capsid that binds more strongly to HSPG as compared to sialic acid or galactose. In some embodiments, the AAV vector comprises an AAV2, AAV3 (including AAV3A or AAV3B), AAV6, AAV13 or AAV-DJ capsid.

In some embodiments, the downstream purification processing step comprises chromatography. In some embodiments, the chromatography is affinity chromatography, pseudoaffinity chromatography, anion exchange chromatography, cation exchange chromatography, hydrophobic interaction chromatography, or size exclusion chromatography.

In some embodiments, the downstream purification process is affinity chromatography to produce an affinity chromatography pool and wherein the affinity chromatography pool comprises at least 1×10¹³ vg/liter host cell culture (vg/L) or a range from about 1×10¹³ vg/L to about 5×10¹⁴ vg/L or more.

In some embodiments, the downstream purification process is anion exchange chromatography to produce an anion exchange chromatography pool and wherein the anion exchange chromatography pool comprises at least 1×10¹³ vg/liter host cell culture (vg/L) or a range from about 1×10¹³ vg/L to about 5×10¹⁴ vg/L or more.

In some embodiments, no endonuclease is added to the lysate. In some embodiments, the volume of the cell suspension prior to lysis is at least 100 L.

In some aspects, a biological product is produced by a method of the disclosure. In some embodiments, the biological product is a recombinant viral vector for expressing a heterologous gene selected from the group consisting of: an adenovirus vector, an adeno-associated virus (AAV) vector, a retrovirus vector, or a lentivirus vector. In some embodiments, the biological product is an AAV vector.

In some aspects, a composition comprises an AAV vector produced by a method of the disclosure. In some embodiments, the capsids in said AAV vector composition are at least 20%, 30%, 40%, 50%, 60%, 70%, 80%, or 90% full capsids. In some embodiments, the composition comprises not more than about 200, 150, 100, 90, 80, 70, 60, 50, 45, 40, 35, 30, 25, or 20 pg/1×10⁹ vg of host cell DNA.

### DETAILED DESCRIPTION OF THE INVENTION

Described in detail below are exemplary non-limiting embodiments of various methods, compositions, and systems which can usefully be employed to remove host cell DNA from crude host cell lysates without resorting to use of endonucleases or chemicals such as octoxynol-9 or domiphen bromide. Although an advantage of the methods described herein is effective removal of host cell DNA without use of octoxynol-9 or domiphen bromide, use of such chemicals if desired is not foreclosed in certain embodiments of the methods.

According to certain embodiments, the disclosure provides methods of lysing a preparation or sample of host cells which have produced a desired biological product, precipitating at least a portion of the host cell DNA released from the cells to form a flocculant and a supernatant, and in some embodiments, filtration of the flocculant and supernatant to produce a clarified lysate. In some embodiments, before host cells are lysed, they are grown or maintained in culture for a time and under conditions sufficient to produce the desired biological product. In other embodiments, after filtration to produce the clarified lysate, the desired biological product is at least partially purified from the supernatant.

### Definitions

As used herein, the term “eluate” refers to fluid exiting from a chromatography stationary phase (e.g., a monolith, membrane, resin, media) (e.g., “eluting from the stationary phase”) comprised of mobile phase and material that passed through the stationary phase or was displaced from the stationary phase. In some embodiments, a stationary phase includes, for example, a monolith, a membrane, a resin or a media. The mobile phase may be a solution that has been loaded onto a column and has flowed through the column (i.e., “flow-through fraction”); an equilibration solution (e.g. an equilibration buffer); an isocratic elution solution; a gradient elution solution; a solution for regenerating a stationary phase; a solution for sanitizing a stationary phase; a solution for washing; and combinations thereof.

As used herein, the term “flocculation” refers to the process by which fine particulates are caused to clump together into a “floc” or a “flocculant”. The fine particles may include proteins, nucleic acids, cellular fragments resulting from lysis of host cells. In some embodiments, a floc that forms in a liquid phase may float to the top of the liquid (creaming), settle to the bottom (sedimentation) of the liquid or be filtered from the liquid phase.

rAAV vectors are referred to as “full,” a “full capsid,” “full vector” or a “fully packaged vector” when the capsid contains a complete, or essentially complete, vector genome, including a transgene. During production of rAAV vectors by host cells, vectors may be produced that have less packaged nucleic acid than the full capsids and contain, for example a partial or truncated vector genome. These vectors are referred to as “intermediates,” an “intermediate capsid,” a “partial” or a “partially packaged vector.” An intermediate capsid may also be a capsid with an intermediate sedimentation rate, that is a sedimentation rate between that of full capsids and empty capsids, when analyzed by analytical ultracentrifugation. Host cells may also produce viral capsids that do not contain any detectable nucleic acid material. These capsids are referred to as “empty(s),” or “empty capsids.” Full capsids may be distinguished from empty capsids based on A260/A280 ratios determined by SEC-HPLC, whereby the A260/A280 ratios have been previously calibrated against capsids (i.e., full, intermediate and empty) isolated by preparative ultracentrifugation. Other methods known in the art for the characterization of capsids include CryoTEM, capillary isoelectric focusing and charge detection mass spectrometry. Calculated isoelectric points of ˜6.2 and ˜5.8 for empty and full AAV9 capsids, respectively have been reported (Venkatakrishnan et al., J. Virology (2013) 87.9:4974-4984).”

As used herein, the term “host cell DNA,” “HCD” or “HCDNA” refers to residual DNA, derived from a host cell culture which produced a rAAV vector, and present in a cell lysate, clarified lysate, chromatography fraction (e.g., an affinity eluate, an AEX eluate, a wash) or a chromatography load (e.g., an affinity load, an AEX load). Host cell DNA may be measured by methods know in the art such as qPCR to detect a sequence unique to the host cells. General DNA concentrations may be estimated using fluorescence dyes (e.g. PicoGreen® or SYBR® Green), absorbance measurement (e.g. at 260 nm, or 254 nm) or electrophoretic techniques (e.g. agarose gel electrophoresis, or capillary electrophoresis). An amount of HCDNA present in an eluate may be expressed relative to the amount of vg present in a solution, for example, ng HCDNA/1×10¹⁴ vg or pg HCDNA/1×10⁹ vg. An amount of HCDNA present in a solution may be expressed relative to the amount of vg present in a volume of the solution, for example, pg HCDNA/mL.

As used herein, the term “host cell protein” or “HCP” refers to residual protein, derived from a host cell culture which produced a rAAV vector, present in a cell lysate, clarified lysate, chromatography fraction (e.g., an affinity eluate, an AEX eluate, a wash) or a chromatography load (e.g., an affinity load, an AEX load). Host cell protein may be measured by methods known in the art, such as ELISA. Host cell protein can be semi-quantitatively measured by various electrophoretic staining methods (e.g., silver stain SDS-PAGE, SYPRO® Ruby stain SDS-PAGE, and/or Western blot). An amount of HCP present in a solution may be expressed relative to the amount of vg present, for example, ng HCP/1×10¹⁴ vg or pg HCP/1×10⁹ vg.

As used herein, the term “therapeutic polypeptide” is a peptide, polypeptide or protein (e.g., enzyme, structural protein, transmembrane protein, transport protein) that may alleviate or reduce symptoms that result from an absence or defect in a protein in a target cell (e.g., an isolated cell) or organism (e.g., a subject). A therapeutic polypeptide or protein encoded by a transgene is one that confers a benefit to a subject, e.g., to correct a genetic defect, to correct a deficiency in a gene related to expression or function. Similarly, a “therapeutic transgene” is the transgene that encodes the therapeutic polypeptide. In some embodiments, a therapeutic polypeptide, expressed in a host cell, is an enzyme expressed from a transgene (i.e., an exogenous nucleic acid that has been introduced into the host cell). In some embodiments, a therapeutic polypeptide is a copper-transporting ATPase 2 protein, or fragment thereof, expressed from a therapeutic transgene transduced into a liver cell.

As used herein, the term “transgene” is used to mean any heterologous polynucleotide for delivery to and/or expression in a host cell, target cell or organism (e.g., a subject). Such “transgene” may be delivered to a host cell, target cell or organism using a vector (e.g., rAAV vector). A transgene may be operably linked to a control sequence, such as a promoter. It will be appreciated by those of skill in the art that expression control sequences can be selected based on an ability to promote expression of the transgene in a host cell, target cell or organism. Generally, a transgene may be operably linked to an endogenous promoter associated with the transgene in nature, but more typically, the transgene is operably linked to a promoter with which the transgene is not associated in nature. An example of a transgene is a nucleic acid encoding a therapeutic polypeptide, for example a copper-transporting ATPase 2 polypeptide or fragment thereof, and an exemplary promoter is one not operable linked to a nucleotide encoding copper-transporting ATPase 2 in nature. Such a non-endogenous promoter can include an alpha-1-antitrypsin promoter or a liver specific promoter, among many others known in the art.

As used herein, the term “vector genome” refers to a nucleic acid that is packaged/encapsidated in an AAV capsid to form a rAAV vector. Typically, a vector genome includes a heterologous polynucleotide sequence (e.g., a transgene, regulatory elements, etc.) and at least one ITR. In cases where a recombinant plasmid is used to construct or manufacture a recombinant vector (e.g., rAAV vector), the vector genome does not include the entire plasmid but rather only the sequence intended for delivery by the viral vector. This non-vector genome portion of the recombinant plasmid is referred to as the “plasmid backbone,” which is important for cloning, selection and amplification of the plasmid, a process that is needed for propagation of recombinant viral vector production, but which is not itself packaged or encapsidated into a rAAV vector. Typically, the heterologous sequence to be packaged into the capsid is flanked by the ITRs such that when cleaved from the plasmid backbone, it is packaged into the capsid.

### Host Cells

As used herein, “host cells” means cells suitable for or adapted to in vitro production of desired biological products. Host cells are often clonal cell lines capable of dividing for multiple generations before senescence stops growth, or may even be immortal. For use in the methods of the disclosure, host cells can be modified, transiently or non-transiently, through the introduction of exogenous genetic information designed to direct biosynthesis in host cells of specific biological products (e.g. a rAAV vector). For example, host cells can be transfected with nucleic acid containing a nucleobase sequence encoding a protein or regulatory RNA (such as IncRNA, miRNA, or siRNA). In some embodiments, the nucleic acid is DNA, such as a plasmid in which the coding sequence is under the control of a transcriptional regulatory element, such as a promoter and enhancer, that can be acted on by the cellular transcription and splicing machinery to produce mRNA. In other embodiments, a nucleic acid can be RNA, such as mRNA, capable of being directly translated into protein.

Various ways are known in the art for transfecting host cells with DNA or RNA. These include, without limitation, mixing DNA or RNA with certain compounds that can complex with nucleic acids and then be taken up into the cells, including calcium phosphate or cationic organic compounds, such as DEAE-dextran, polyethylenimine (PEI), polylysine, polyornithine, polybrene, cyclodextrin, cationic lipids, and others known in the art. Transfection can also be performed non-chemically via electroporation and more exotic technologies, such as biolistic particle delivery. As known in the art, transfection can be transient or stable. With transient transfection, the transfected DNA or RNA exists in the cell for a limited period of time and, in the case of DNA, does not integrate into the genome. With stable transfection, DNA introduced into the cell can persist for long periods either as an episomal plasmid, or integrated into a chromosome. Usually, to produce stably transfected cells, a plasmid containing a selection marker, as well as the gene or genes for expressing the desired biological product, is transfected into the cells which are then grown and maintained under selective pressure, i.e., conditions that kill non-transfected cells or transfected cells from which the exogenous DNA, including its selection marker, are lost for some reason. For example, plasmids can contain an antibiotic resistance gene and transfected cells can be selected for by adding the antibiotic to the media in which the cells are grown. In some embodiments, the gene for producing the biological product introduced into stably transfected host cells is under the control of an inducible promoter and is not expressed, or only at a low level, unless an environmental factor, such as a drug, metal ion, or temperature increase, which induces the promoter, is introduced as the cells are grown.

In other embodiments, host cell genomes can be modified in a non-transient and targeted fashion using genetic engineering methods, such as knock-in, or gene editing methods, to direct host cells to produce desired biological products, components thereof, or other gene products necessary for the biosynthesis of such products. The invention is not limited by the manner in which host cells are generated. Foreign genes can also be introduced into host cells for purposes of directing production of desired biological products by transduction, in which host cells are infected with modified viruses (i.e., vectors) containing such genes. Examples of viral vectors useful for such purposes include adenovirus, retroviruses (including lentiviruses), baculoviruses, vaccinia virus, and herpes simplex virus, with others being possible.

Host cells can be any type of cell known in the art to be useful for the purpose of biosynthesizing desired biological products. Host cells can be prokaryotic cells, such as bacteria, such as E. coli, or eukaryotic cells, such as fungal cells, such as yeast cells, such as plant cells, or such as animal cells, such as insect cells or mammalian cells, including rat, mouse, or human cells. In some embodiments, host cells useful in the methods of the disclosure are mammalian host cells, examples of which include HeLa cells, COS cells, HEK293 cells (and variants of HEK293 cells, such as HEK293E, HEK293F, HEK293H, HEK293T or HEK293FT cells), A549 cells, BHK cells, Vero cells, NIH 3T3 cells, HT-1080 cells, Sp2/0 cells, NS0 cells, C127 cells, AGE1.HN cells, CAP cells, HKB-11 cells, or PER.C6 cells, with many others being possible. In some embodiments, host cells useful in the methods of the disclosure are insect host cells, examples of which include Sf9 cells, ExpiSf9, Sf21 cells, S2 cells, D.Mel2 cells, Tn-368 cells, or BTI-Tn-5B1-4 cells, with many others being possible.

For purposes of producing biological products, host cells are often grown or maintained in culture under controlled conditions conducive to their growth to relatively high density and the biosynthesis of the desired biological product. For example, host cells can be grown in liquid media of defined chemical composition that provides all the nutrients necessary for cell growth and biosynthesis. Exemplary media includes DMEM, DMEM/F12, MEM, RPMI 1640, for mammalian host cells, and Express Five SFM, Sf-900 II SFM, Sf-900 III, or ExpiSf CD, for certain insect cells. Such media may be supplemented with antibiotics, growth factors or cytokines (produced recombinantly or present in animal serum, such as FBS) known to stimulate growth of the particular type of cells in use, as well as other ingredients that may be required for optimal biosynthesis and/or activity of a desired biological product, but that would otherwise be in limiting supply. Exemplary supplements include essential amino acids, glutamine, vitamin K, insulin, BSA, or transferrin. In addition to the growth media, other culture conditions may be controlled to optimize growth and/or productivity of the cells, such as pH, temperature and CO₂ and oxygen concentration.

Host cells in culture can be grown or maintained in many containers known in the art, such as stirred tank bioreactors, wave bags, spinner flasks, hollow fiber bioreactors, or roller bottle, some of which can be designed and configured for single use or multiple use. Depending on the characteristics of the host cells in question, host cells can be grown in adherent cell culture, where the cells attach to and grow while in contact with a physical substrate, or in suspension cell culture, either where single cells float free in the media that sustains them, or while attached to bead microcarriers, which are suspended in the media. As known in the art, various technologies have been developed and can be used to grow host cells to high cell density, such as perfusion culture.

As known in the art, samples of host cells are often maintained in frozen cell banks, such as master cell banks and working cell banks, which facilitate production of biological products in many batches over time, while ensuring consistent performance by the host cells. Before a campaign to produce a biological product, a frozen sample of host cells from a cell bank would typically be thawed, seeded into a small culture volume, and grown to ever higher densities or numbers in cultures of increasing volume. When host cells have reached a desired cell density and/or volume in culture, exogenous genetic material can be introduced, such as by transfection with plasmid DNA or infection with viral vectors, to cause them to begin producing the desired biological product. Or, if using non-transiently modified host cells in which the genes for the biological product are under inducible control, the environmental factor necessary to induce expression can be introduced. Host cells can then be grown or maintained in culture for time and under conditions sufficient for them to produce a desired amount of the biological product (e.g., a rAAV vector). In some embodiments, HEK293 cells are grown, maintained or both, in culture for a time and under conditions sufficient for them to produce a desired amount of rAAV vector for the treatment of a disease such as Wilson disease, Gaucher disease, Duchenne muscular dystrophy, Hemophilia A or Hemophilia B.

### Biological Products

The methods of the disclosure can usefully be employed in the production of any biological product capable of being made by a host cell, a significant portion of which is retained within host cells with intact cell membranes. Non-limiting examples include recombinant proteins of any kind, including monoclonal antibodies of any type and specificity, clotting factors, enzymes (whether for use as therapeutics or in industrial applications), growth factors, hormones, cytokines, antigenic proteins to serve as vaccines, and any naturally occurring or non-naturally occurring versions or variants of any of the foregoing, including versions that are fused with heterologous protein regions or domains, such as fusion of a clotting factor with albumin, or an Fc region from an immunoglobulin protein. Such proteins can include any post-translational modification known to those of skill in the art, such as covalent addition of carbohydrate groups, lipid molecules, and non-standard amino acids. Such proteins can also comprise a plurality of polypeptide chains, which can be covalently or non-covalently bound to each other. In other embodiments, biological products can be supramolecular assemblies, such as viruses, or modified viruses engineered to kill cancer cells (oncolytic viruses) or to serve as vectors of heterologous genes, for example as vectors to be used in gene therapy (e.g., rAAV vectors). Non-limiting examples include adenovirus, vaccinia virus, lentiviruses, and adeno-associated viruses, or vectors made using such viruses.

### Adeno-Associated Viral (AAV) Vectors

According to some embodiments, the methods of the disclosure are useful in the production of adeno-associated virus (AAV) which has been recombinantly modified to function as a viral vector for gene therapy (thus, an AAV vector or a rAAV vector). So modified, AAV vectors are capable of delivering gene cassettes, often including regulatory elements for the appropriate initiation and termination of gene transcription, into targeted cells via transduction. In this way, AAV vectors can supply a functional copy of a gene to a target cell in which the endogenous version is missing or mutated.

As is well known in the art, AAV is a small non-enveloped, apparently non-pathogenic virus that depends on certain other viruses to supply gene products, known as helper factors, essential to its own replication, a quirk of biology that has made AAV well-suited to serve as a recombinant vector. For example, adenovirus (AdV) can serve as a helper virus by providing certain adenoviral factors, such as the E1A, E1B55K, E2A, and E4ORF6 proteins, and the VA RNA, in cells co-infected by adenovirus and AAV. Numerous types of AAV have been discovered which are restricted in their ability to infect certain animals (such as mammal and bird) and species (such as human and rhesus monkey), and having a tendency within species to infect certain tissues (such as liver or muscle) more so than others, a phenomenon called tropism, based on specific binding to different cell surface receptors. One type of AAV that infects humans, called AAV2, is particularly well characterized biologically, although many other types have found utility in creating gene therapy vectors.

In nature, the AAV genome is a single strand of DNA, about 4.7 kilobases long in AAV2, which contains two genes called rep and capsid (cap). By virtue of alternative splicing of the transcripts from two promoters, the rep gene produces four related multifunctional proteins called Rep (Rep 78, Rep 68, Rep 52 and Rep 40 in AAV2) which are involved in genome replication and packaging, and gene expression. Alternative splicing of the transcript from the single promoter controlling the cap gene produces three related structural proteins, VP1, VP2, and VP3, a total of 60 of which self-assemble to form the virus's icosahedral capsid in a ratio of approximately 1:1:10, respectively. VP1 is the longest of the three VP proteins, and contains amino acids in its amino terminal region that are not present in VP2, which in turn is longer than VP3 and contains amino acids in its amino terminal region that are not present in VP3. The capsid protects the AAV genome, and also is responsible for binding specifically to receptors on the surface of target cells.

In addition to the rep and cap genes, intact AAV genomes have a relatively short (145 nucleotides in AAV2) sequence element positioned at each of their 5′ and 3′ ends called an inverted terminal repeat (ITR). ITRs contain nested palindromic sequences that can self-anneal through Watson-Crick base pairing to form a T-shaped, or hairpin secondary structure. In AAV2, ITRs have important functions required for the viral life cycle, including converting the single stranded DNA genome into double stranded form required for gene expression, as well as packaging by Rep proteins of single stranded AAV genomes into capsid assemblies.

After an AAV2 virion binds its cognate receptor on a cell surface, the viral particle enters the cell via endocytosis. Upon reaching the low pH of lysosomes, capsid proteins undergo a conformational change which allows the capsid to escape into the cytosol and then be transported into the nucleus. Once there, the capsid disassembles, releasing the genome which is acted on by cellular DNA polymerases to synthesize the second DNA strand starting at the ITR at the 3′ end, which functions as a primer after self-annealing. Expression of the rep and cap genes can then commence, followed by formation of new viral particles.

The relative simplicity of AAV structure and life cycle, and the fact that it is not known to be pathogenic in humans, inspired investigators to engineer AAV and convert it from a virus to a recombinant vector for gene therapy. Briefly, this was done by cloning the entire genome of AAV2, including both ITRs, into a plasmid, removing the rep and cap genes into a separate plasmid, and replacing them with a gene expression cassette comprising a heterologous transcription control region (promoter and optionally an enhancer) and gene of interest (which is sometimes referred to as a transgene). Thus, the only viral genome sequences retained in the vector genome are the ITRs due to their critical function in packaging and gene expression, without which AAV vectors could not be produced or function to express the gene of interest after transduction of target cells. Finally, to avoid the need for co-infection with a helper virus, genes for the so-called helper factors (such as, in the case of AdV, the early region 1A (E1A), E1B55K, E2A, E4orf6, and viral associated (VA) RNA helper factors) were cloned into a third plasmid. When the three plasmids are replicated to high number in bacteria, purified and transfected together into mammalian cells, such as HEK293 cells, Rep and VP proteins, and the AdV helper factors are expressed from their respective plasmids and function in the cells to assemble capsids, and package into them single stranded vector genomes replicated from the plasmids on which its sequence resides. Because the rep and cap genes exist in trans on a different plasmid, outside their usual context flanked by ITRs, they are not packaged into the vectors. Consequently, while vectors are able to bind to target cells and convey the expression cassette within their genomes into the cells, they cannot replicate and create new vector particles. For this reason, the term “transduction” is often used to refer to this process in place of the term “infection.” If the vector functions as intended, the expression cassette will be transcriptionally active and produce the gene product encoded by the gene of interest.

For use in connection with the methods of the disclosure, an AAV vector can include any gene of interest within an AAV vector genome of any sequence, structure, arrangement of functional sub-elements, and configuration known in the art to be suitable for its intended use, such as use in gene therapy. As AAV vectors are typically designed, choice of the gene of interest is limited only by the packaging capacity of the capsid, so that the gene's length when combined with all other elements in the genome required for vector function, such as the transcriptional regulatory region and the ITRs, does not exceed approximately 5 kilobases in the case of AAV2, although experimental strategies have been developed to surpass the packaging limit.

For purposes of gene therapy, the gene of interest can be any gene, the product of which would be understood to prevent or treat, but not necessarily cure, any disease or condition. In some embodiments, gene therapy is intended to prevent or treat a disease or condition characterized by an abnormally low amount or even absence of a product produced by a naturally occurring gene, such as might occur due to a loss of function mutation. Relating to such embodiments, the gene of interest can be one intended to compensate for the defective gene by providing the same or similar gene product when expressed. A non-limiting example would be a vector designed to express a functional version of clotting factor IX for use in gene therapy of hemophilia B, which is caused by a loss of function mutation in the native factor IX gene. In another embodiment, a vector may be designed to express a functional version of a copper transporting ATPase 2 polypeptide for use in gene therapy of Wilson disease (see, WO2016/097219, WO2016//097218). In another embodiment, a vector may be designed to express a functional version of a dystrophin polypeptide for use in gene therapy of Duchenne muscular dystrophy (see, WO 2017/221145).

In other embodiments, however, the gene of interest could be one intended to counteract the effects of a deleterious gain of function mutation in targeted cells. In some embodiments, the gene of interest can encode a transcriptional activator to increase the activity of an endogenous gene which produces a desirable gene product, or conversely a transcriptional repressor to decrease the activity of an endogenous gene which produces an undesirable gene product. In some embodiments, the gene of interest can encode for a protein, or an RNA molecule with a function distinct from encoding protein, such as a regulatory non-coding RNA molecule (e.g., micro RNA, small interfering RNA, piwi-acting RNA, enhancer RNA, or long non-coding RNA). Protein coding sequences in a gene of interest can be codon-optimized, and translation start sites (e.g., Kozak sequence) can be modified to increase or decrease their tendency to initiate translation. In some embodiments, the gene of interest can contain one or more open reading frames. In other embodiments, a vector genome can comprise more than one gene of interest, each part of its own separate transcriptional unit, or different products can be produced from a single transcriptional unit by inclusion of alternative splice sites.

Apart from the gene of interest, many other aspects of AAV vector genomes are amenable to design choice and optimization depending on the intended use of the vector. Without limitation, the transcriptional control region can be constitutively active, tissue specific, or inducible, and can include a promoter as well as one or more enhancer elements. A transcriptional control region can comprise the same nucleotide sequence as would occur in a gene naturally, or be modified to improve its function and/or reduce its length by changing, adding or removing nucleotides relative to a sequence found in nature, or even be entirely synthetic. In other embodiments, vector genomes can further comprise untranslated regions from the 5′ and/or 3′ end of genes, non-coding exons, introns, transcriptional termination signals (e.g., polyA signal sequence), elements that stabilize RNA transcripts, splice donor and acceptor sites, lox sites, binding sites for regulatory miRNAs, elements that enhance nuclear export of mRNAs, such as the woodchuck hepatitis virus post-transcriptional regulatory element (WPRE), and any other element demonstrated empirically to improve expression of the gene of interest, even if the mechanism may be uncertain.

In some embodiments, a vector genome can be designed for purposes of editing or otherwise modifying the genome of a target cell. For example, a vector genome can include a gene of interest flanked by homology arms intended to promote homologous recombination between the vector genome and the target cell genome. In another example, a vector genome can be designed to carry out CRISPR gene editing by expressing a guide RNA (gRNA) and/or an endonuclease, such as Cas9 or related endonucleases, such as SaCas9, capable of binding the gRNA and cleaving a DNA sequence targeted by the gRNA.

As known in the art, the ITRs typically used in AAV vectors originate from AAV2, but ITRs derived from other serotypes and naturally occurring AAV isolates, or hybrid, or even entirely synthetic ITRs, may be used as well. In some embodiments, vector genomes include two intact ITRs, one at each end of the single stranded DNA genome. In other embodiments, however, a third mutated ITR lacking a terminal resolution site can be positioned in the center of the genome, such as occurs in so-called self-complementary AAV (scAAV) genomes, which can self-anneal after capsid uncoating into double stranded form, permitting gene expression to proceed immediately without need for second strand synthesis, as is the case with conventional single stranded AAV genomes. ITRs from one type of AAV may be used in a genome that is contained in a capsid from the same type of AAV, or in a capsid from a different type of AAV, which are sometimes known as pseudotyped vectors. For example, AAV2 ITRs may be used in a genome that is encapsidated by an AAV2 capsid, or an AAV5 capsid (which is sometimes denoted AAV2/5) or another AAV capsid different from AAV2.

Just as there is wide latitude in the design of vector genomes, AAV vectors can be made using many different naturally occurring and modified AAV capsids. At one time, only six types of primate AAV had been isolated from biological samples (AAV1, AAV2, AAV3, AAV4, AAV5, and AAV6), the first five of which were sufficiently distinct structurally to be classified as different serotypes based on antibody cross reactivity experiments. Later, two novel AAVs, called AAV7 and AAV8 were discovered by PCR amplification of DNA from rhesus monkeys using primers targeting highly conserved regions in the cap genes of the previously discovered AAVs (Gao, G, et al., PNAS (USA) (2002) 99(18):11854-11859). Subsequently, a similar approach was used to clone numerous novel AAVs from human and non-human primate tissues, vastly expanding the scope of known AAV cap protein sequences (Gao, G, et al., J Virol. (2004) 78(12):6381-6388). Many AAV cap protein sequences are highly similar to each other, or previously identified AAVs, and while often referred to as distinct AAV “serotypes,” not all such capsids would necessarily be expected to be immunologically distinguishable if tested by antibody cross reactivity.

Research has established that different AAV capsids have different tissue tropisms, as well as other properties that may make one capsid preferable over another for particular applications. For example, depending on which population is being tested, humans may have high neutralizing antibody titers as a result of exposure to naturally occurring AAVs, which can interfere with the ability of AAV vectors with the same or similar capsids to transduce target cells. Thus, in designing a vector for gene therapy, choice of capsid may in some cases be guided by the immunogenicity of the capsid, and/or the seroprevalence of the patients to be treated.

For use in connection with the methods of the disclosure, an AAV vector can include any capsid known in the art to be suitable for its intended use, such as use in gene therapy. Such capsids include those from naturally occurring AAVs, as well as modified or engineered capsids. For example, naturally occurring capsids can be modified by inserting peptides, or making amino acid substitutions, in the cap protein sequence intended to improve capsid function in some way, such as tissue tropism, immunogenicity, stability, or manufacturability. Other examples include novel capsids with improved properties created by swapping amino acids or domains from one known capsid to another (which are sometimes known as mosaic or chimeric capsids), or which are generated and selected employing DNA shuffling and directed evolution methods. In some embodiments, AAV vectors that can usefully be produced by host cells and purified with the methods of the disclosure include those that use any of the following capsids: AAV1, AAV2, AAV3 (including AAV3A and AAV3B), AAV4, AAV5, AAV6, AAV7, AAV8, AAV9, AAV10, AAV12, AAV13, AAVrh8, AAVRh10, AAV rh32.22, AAVRh39, AAVRh43, Rh74, AAV-DJ, AAV-PHP.B, Anc80, AAV1.1, AAV2.5, AAV6.1, AAV6.2, AAV6.3.1, AAV9.45, AAV2i8, AAVShH10, HSC15/17, RHM4-1, RHM15-1, RHM15-2, RHM15-3/RHM15-5, RHM15-4, RHM15-6, AAVhu.26, AAV29G, AAV-LK03, AAV2-TT, AAV2-TT-S312N, AAV3B-S312N, avian AAV, bovine AAV, canine AAV, equine AAV, primate AAV, non-primate AAV, and ovine AAV, NP4, NP22, NP66, AAVDJ/8, AAVDJ/9 with many others being possible.

As noted above, AAV infection begins when a virion's capsid binds specifically to a receptor on the surface of a target cell. The virion is then taken into the cell via endocytosis, and trafficked to the nucleus, where the capsid uncoats to reveal the genome. Different capsids have been shown to bind specifically to different cellular receptors, which may explain, at least in part, the different tissue tropisms that have been observed among different AAV serotypes and variants. Initial attachment by AAV to cells in many cases appears to involve binding by capsids to glycan moieties displayed on cell surface proteins, with other cell surface proteins playing an important role as co-receptors involved with viral entry. For example, AAV1, AAV5, and AAV6 have been shown to bind to N-linked sialic acid, AAV4 to O-linked sialic acid, and AAV9 to terminal N-linked galactose. Other capsids have been shown to bind specifically to heparan-sulfate proteoglycan (HSPG), including AAV2, AAV3 (including AAV3A and AAV3B), AAV6, AAV13, and AAV-DJ. The cell surface protein known as AAVR is apparently required for entry by a number of AAV serotypes, but other proteins, such as certain integrins and laminin receptor may also be involved depending on the capsid serotype (Huang, LY, Curr. Opin. Virol. (2014) 7:108-118; Zhang, R, et al., Nat. Comms. (2019) 10:3760; Havlik, LP, J. Virol. (2021) 95(19):e00587-21).

In some embodiments, AAV vectors comprising any known or as yet uncharacterized capsid can be purified with the methods of the disclosure. In other embodiments, AAV vectors that can be purified with the methods of the disclosure comprise a capsid that binds more strongly HSPG. Thus, in some embodiments, AAV vectors comprising capsids from AAV2, AAV3 (including AAB3A and AAV3B), AAV6, AAV13, and AAV-DJ can be purified with the methods of the disclosure, whereas in other embodiments, AAV vectors comprising capsids from AAV1, AAV4, AAV5, and AAV9 can be purified with the methods of the disclosure. Specific binding affinity or avidity of a capsid to receptors of any kind can be determined using any technique familiar to those of ordinary skill in the art, such as surface plasmon resonance, or other methods. In some embodiments, AAV vectors that can be purified with the methods of the disclosure comprise a capsid that binds more strongly to HSPG as compared to sialic acid or galactose by a factor of at least or about 2, 3, 4, 5, 6, 7, 8, 9, 10, 20, 50, 100, 250, 500, 1000 times or more, or some other factor.

As known in the art, viral vectors can be produced, including at large scale (e.g., in a bioreactor with a volume of 2000 L or more), in a number of ways. AAV vectors, for example, can be made in mammalian or insect cells and then purified. The traditional approach that does not rely on coinfection with a helper virus involves use of three plasmids, as discussed above. One plasmid contains genes for helper virus factors, a second contains the AAV genome sequence in double stranded form, and the third contains AAV rep and cap genes. The rep/cap plasmid often contains a rep gene from AAV2, although this is not a requirement, and the cap gene sequence is chosen based on which AAV cap protein is desired to constitute the capsid. In practice, the three plasmids are often separately replicated in bacteria, purified, mixed in solution together in predetermined proportions, and then mixed with a transfection agent. The transfection mixture is then used to transfect suitable mammalian host cells (in adherent or suspension cell culture) which are incubated for sufficient time (e.g., 48 to 72 hours, etc.) and under conditions sufficient for the host cells to express the helper factors and the rep and cap genes, and for AAV vector genome to be replicated from its plasmid template and packaged into capsids. In some embodiments, the host cells are HEK293 cells, which constitutively express AdV helper factors E1A and E1B, such that the helper plasmid only need contain the AdV E2A, E4ORF6, and VA RNA genes. Use of other mammalian host cells that do not produce AdV or other viral helper factor on their own would necessitate use of a helper plasmid containing whichever helper factors are missing or are otherwise required. Although the so-called triple transfection method described above is commonly employed, there is no requirement that the helper factor, and rep and cap genes, be provided on separate plasmids. In principle all these genes could be housed in one plasmid, for example, in which case two plasmids can be used in the transfection.

Seeking more efficient methods of producing AAV vector at large scale, stable cell lines have been created that contain some but not all the components that would otherwise need to be introduced into cells by transient transfection. Packaging cell lines contain stably integrated AAV rep and cap genes. Production of AAV in packaging cells requires them to be transiently transfected with a plasmid containing an AAV vector genome and infected with a helper virus. It is also possible to produce AAV vectors in packaging cells without transfection by first infecting them with an AdV (either wild type or in which the E2b gene is deleted) which supplies AdV E1 gene products, which induce rep and cap expression in the cells, as well as helper factors required for AAV replication, followed by infection with a replication deficient hybrid AdV in which an AAV vector genome replaces the E1 gene in the genome of the hybrid virus. Producer cell lines contain stably integrated AAV rep and cap genes, and also an AAV vector genome. Production of AAV in producer cells requires them to be infected with a helper virus. Packaging and producer cells are described further in, e.g., Martin, J, et al., Hum. Gene Methods (2013), 24:253-269; Gao, G P, et al., Hum. Gene Ther. (1998) 9:2353-62); Martin, J, et al., Hum. Gene Ther. Meth. (2013) 24:253-69; Clement, N and JC Grieger, Mol. Ther. Meth. & Clin. Dev. (2016) 3, 16002 (doi:10.1038/mtm.2016.2). Other cellular systems for producing AAV vectors in mammalian cells, including at commercial scale, are possible.

The baculovirus system has also been employed to produce AAV vector. In this system, Sf9 insect cells are infected with recombinant baculovirus vectors that variously contain the AAV rep and cap genes and the AAV genome. The exogenous genes are expressed, followed by genome packaging into vector particles within the cells. In early versions of the system, each component, rep, cap, and genome, were carried by three separate baculoviruses. Later, modifications were made, such as combining rep and cap into a single baculovirus, so that only two types of baculovirus were required, as well as producing Sf9 cell lines containing stably integrated AAV rep and cap genes, which only require infection with a single type of recombinant baculovirus containing an AAV vector genome. Use of the baculovirus system to produce AAV vector is described further in, e.g., Urabe, M, et al., Hum. Gene Ther. (2002) 13:1935-43; Virag, T, et al., Hum. Gene Ther. (2009) 20:807-17; Smith, R H, et al., Mol. Ther. (2009) 17:1888-96; Mietzsch, M, et al., Hum. Gene. Ther. (2014) 25(3):212-22. Other cellular systems for producing AAV vectors in insect cells, including at commercial scale, are possible.

### Lysing Host Cells and Precipitating Host Cell DNA

A host cell comprising a desired biological product can be lysed by disrupting the cell's plasma membrane and nuclear membrane, permitting the cell's internal contents to make contact with the surrounding medium, while not denaturing the biological product sought to be purified. Lysing host cells produces a crude host cell lysate comprising host cell DNA and biological product (e.g., AAV vector), among other cellular components, dispersed in the fluid in which the cells had been suspended, or which had otherwise surrounded the cells immediately before lysis.

In some embodiments of the disclosure, host cell lysis is effected by contacting the host cells with a detergent in sufficient concentration to cause disruption, dissolution, or lysis of the cells' plasma membrane and nuclear membrane. In some embodiments, host cell lysis can be effected by contacting the host cells with a detergent and mixing (e.g., stirring, rocking) the solution comprising the cells and the solution comprising the detergent. In some embodiments, host cell lysis can be effected by contacting the host cells with a detergent and by use of another method such a mechanical method (e.g., a high pressure homogenizer or bead mill) or a non-mechanical method (e.g., exposing cells to heating, freeze-thaw cycles, osmotic shock, sonication or cavitation) (Islam, M S, et al., Micromachines (2017) 8(83):1-27).

Lysis of host cells releases host cell DNA into the surrounding fluid, for example a physiologically compatible fluid, in which the cells are suspended. A significant proportion of the host cell DNA is genomic DNA, but can include any DNA released from a lysed host cell, for example mitochondrial DNA and/or plasmid DNA. While a goal of the present methods is to reduce the amount of host cell DNA in a sample of lysed host cells, the methods may also be effective, in some embodiments, to at least partially remove RNA from a lysate, as well as proteins, such as histones, complexed with host cell DNA as chromatin. As used herein, the term removal of host cell DNA from lysates of host cells does not require removal of all such DNA, but merely reduction in the amount of host cell DNA in a portion of the lysate. In some embodiments, host cell DNA in a lysate can be precipitated, or flocculated to produce a flocculant.

Methods for lysis of host cells and for precipitation of the released host cell DNA are known in the art and generally comprise the use of at least two reagents, for example a detergent such as octoxynol-9 to lyse the cells and a cationic compound such as domiphen bromide to precipitate the DNA. Advantageously, the methods disclosed herein use a single reagent, CTAB, which lyses host cells and causes precipitation of the released host cell DNA. Without wishing to be bound by theory, these two effects of CTAB occur, if not simultaneously, in rapid succession such that they appear to occur essentially simultaneously. In some embodiments, a second reagent is used, such as a modifier, which increases the efficiency of the cell lysis and DNA precipitation. In some embodiments, an increase in efficiency of cell lysis and DNA precipitation is demonstrated by an increase in vector genome recovery in a clarified lysate or other downstream solution (e.g., affinity pool, AEX pool).

As noted above, host cells may be grown and maintained in adherent cell culture or suspension cell culture. In some embodiments, lysis of host cells grown in adherent cell culture can conveniently be performed in several ways. The growth medium can be removed and then at least a solution comprising a detergent (e.g., CTAB), and optionally a solution comprising another component such as a modifier (e.g., a salt, e.g., MgSO₄), at a final concentration sufficient to cause cell lysis and precipitation of host cell DNA can be added to the container in which the cells are grown and then be caused to make contact with the cells until cell lysis results. The container may be agitated, rocked, etc. to cause effective distribution of the solution(s) over the cells and mixing of the lysed cells and their contents with the solution(s).

Alternatively, a concentrated stock solution comprising a detergent, and optionally another solution comprising a component such as a modifier (e.g., a salt, e.g., MgSO₄), can be prepared and added directly to the growth media (or other physiologically compatible fluid in which the cells are being maintained, such as phosphate buffered saline (PBS), or the like) to a desired final detergent concentration, and optionally a final modifier concentration (e.g., as a % weight by volume, % weight by weight, or molarity) sufficient to cause cell lysis. The media and solution(s) comprising the detergent (e.g., CTAB), and optionally the modifier, can then be mixed and caused to make contact with the adherent cells in the container until cell lysis results.

In other embodiments, host cells grown in adherent cell culture can be chemically or enzymatically detached from their substrate, after which a solution comprising a detergent (e.g., CTAB), and optionally a solution comprising another component such as a modifier (e.g., a salt, e.g., MgSO₄), as described above, can be added to the container and allowed to make contact with cells in suspension until cell lysis and precipitation of the host cell DNA results.

Alternatively, the detached cells can be removed from their growth container and transferred in suspension to a new container where lysis and precipitation of the host cell DNA would be performed, as described above. In some embodiments of the processes described above, the cells are contacted with a solution comprising a detergent followed by an incubation period of at least 1 minute during which the container comprising the cells and detergent solution are mixed (e.g., by rocking), followed by contact of the cells with a solution comprising another component, such as a modifier. In some embodiments of the processes described above, the cells are contacted with a solution comprising a modifier (e.g., a salt) followed by an incubation period of at least 1 minute during which the container comprising the cells and modifier solution are mixed (e.g., by rocking), followed by contact of the cells with a solution comprising a detergent.

In some embodiments, host cells grown or maintained in suspension cell culture can be lysed by adding to the growth media (or other physiologically compatible fluid in which the cells are maintained, such as phosphate buffered saline (PBS), or the like) a concentrated stock solution comprising a detergent, and optionally other components such as a modifier (e.g., a salt), to a desired final detergent and modifier concentration (e.g., as a % weight by volume, % weight by weight, or molarity) sufficient to cause cell lysis. In some embodiments, other components, such as a modifier (e.g., a salt) are added to the growth media before the detergent is added to the growth media. In some embodiments, other components, such as a modifier (e.g., a salt) are added to the growth media after the detergent is added to the growth media. The media and solution(s) can then be mixed to evenly distribute the detergent (and any other components) throughout the culture volume, and allowed to contact the cells until cell lysis and precipitation of the host cell DNA results.

In some embodiments, the host cells are grown or maintained in bioreactors of any desired volume to which a solution comprising a detergent, a solution comprising another component such as a modifier, or both is added as one or more boluses, or continually until the entire desired volume of lysis solution has been added. A detergent solution, a modifier solution or both can be added to a bioreactor, or any container in which host cells are to be lysed, in any way that is known in the art, for example, from above, such as through a tube positioned above the fluid in which the host cells are suspended, or from below the surface of such fluid at any desired level of the bioreactor, such as through subsurface addition lines or tubes. Mixing of the media in which the cells are suspended and the solution(s) can proceed over the entire period during which cells are lysed, or for a shorter period followed by an incubation period in which cells are allowed to lyse without mixing or agitation. Mixing can be performed in any way that is known the art, such as using impellers, pumps or by rocking. In some embodiments, the host cells and the media in which they were grown in suspension culture can be separated, such as by allowing host cells to settle out, and the media removed and replaced with a different fluid, such as fresh media of the same or different kind, or some other physiologically compatible fluid, to which a detergent solution, a modifier solution or both is then added followed by mixing and cell lysis. This can occur in the same container in which the cells were grown or in a new container of any suitable size. The modifier solution may be added to a physiologically compatible fluid comprising host cells followed by a period of mixing (e.g., rocking for at least 1 minute) followed by addition of the detergent solution followed by a period of mixing (e.g, rocking for at least 1 minute) and allowed to contact the cells until cell lysis and precipitation of the host cell DNA results. The detergent solution may be added to a physiologically compatible fluid comprising host cells followed by a period of mixing (e.g., rocking for at least 1 minute) followed by addition of the modifier solution followed by a period of mixing (e.g, rocking for at least 1 minute) and allowed to contact the cells until cell lysis and precipitation of the host cell DNA results.

In some embodiments, a solution comprising MgSO₄ may be added to a physiologically compatible fluid comprising host cells followed by a period of mixing (e.g., rocking for at least 1 minute) followed by addition of a solution comprising the detergent cetyltrimethylammonium bromide (CTAB) followed by a period of mixing (e.g., rocking for at least 1 minute) and allowed to contact the cells until cell lysis and precipitation of the host cell DNA results.

In some aspects, methods of host cell lysis is effected by contacting the host cells with CTAB in sufficient concentration to cause disruption, dissolution, or lysis of the cells' plasma membrane and nuclear membrane and precipitation of host cell DNA. CTAB is a quaternary amine compound and is used in industrial and laboratory applications for DNA purification and polysaccharide vaccine production (Lever et al., Front. Microbiol. 19 May 2015 (doi:10.3389/fmicb.2015.00476); Shi, L., Internat. J. Biol. Macromol. (2016) 92: 37-48).

Belonging to the same chemical class as domiphen bromide, CTAB is capable of precipitating DNA in the presence of low amounts of salt (e.g., <0.5 M NaCl). Protocols have been developed to achieve high quality DNA preparations of nuclear and plasmid DNA from plant sources (Nadia Aboul-Ftooh Aboul-Maaty et al., Bulletin of the National Research Center (2019) 43:25).

CTAB has also been used as a purification agent in the downstream processing of polysaccharide vaccines. Long chain quaternary ammonium salts such as CTAB, can form coordination compounds with acidic polysaccharides or long chain high molecular weight polysaccharides. This type of coordination compound is not soluble in aqueous solutions of low ionic strength (Karamanos et. al., Carbohydrate Res (1992) 231:197-204) but can be gradually dissociated by increasing ionic strength of solution. Vaccines have been purified by precipitation with CTAB at 1% w/v final concentration followed by extraction with ethanol or dissolution in saline. Sodium deoxycholate (DOC) precipitation was used to remove host cell protein impurities, followed by ethanol precipitation and chromatography to remove any additional impurities (Riza et al., Proceeding International Seminar Chem. (2008) 294-296). CTAB may also be used in the downstream processing of viral vaccines.

Affinity of CTAB to polysaccharides has also lead to development of several analytical methods for detection of the highly sulfated and negatively charged acidic linear polysaccharide heparin in various matrices, including human plasma and has relied on the competitive binding of CTAB (Faham et al., 2017, J Applied Spectroscopy (2017) 84(3):425).

CTAB has been used to precipitate cellular DNA from adenoviral containing cell lysates, but was shown to drive down Ad5 titer significantly alongside host cell DNA concentration (Goerke et al., Biotechnology Bioengineering (2005) 91(1):12-21). In this study, cell lysis relied upon multiple freeze-thaw cycles or 0.1% Triton X-100 and 0.05% polysorbate 80 as lysis detergents. Goerke et al. found that domiphen bromide more selectively precipitated the host cell DNA and was the preferred precipitant for use in AD5 purification.

As disclosed herein, the inventors recognized that use of cell lysis detergents such as octoxynol-9 (i.e., Triton X-100) to produce a cell lysate for purification of some AAV vectors often resulted in reduced vector recovery as the majority of the octoxynol-9 released AAV vector bound (via the capsid) to host cell debris and was not recovered in the clarified harvest filtrate. The inventors also recognized that positively charged CTAB, and its ability to precipitate polysaccharides, could be used to simultaneously lyse host cells, precipitate host cell DNA and reduce or block non-specific binding of host cell debris comprising negatively charged HSPG to AAV vectors (e.g., rAAV3B vectors) produced by the host cells, such that the vectors could be recovered in the clarified harvest filtrate.

In some embodiments, particularly when host cells are grown or maintained in suspension culture, the step of host cell lysis and DNA precipitation can be performed at a predetermined viable cell density, meaning the number of viable host cells in a defined volume of media, or other physiologically compatible fluid in which they are suspended, for example viable cells per milliliter (vc/mL). Cell viability can be determined using any technique known in the art. For example, a sample of cells can be withdrawn from the culture in which they are grown or maintained, mixed with a vital dye such as trypan blue, and then the total number of cells excluding the dye counted on a hemocytometer from which the number of viable cells per mL (or any other volume) can readily be calculated. Alternatively, viable cell density can be monitored in real time using sensors, such as permittivity sensors, more information about which can be found, e.g., in Metze, S, et al., Bioprocess Biosys. Eng. (2020) 43:193-205 (2020).

In some embodiments, host cells producing a desired biological product, such as an AAV vector, are lysed (harvested) at a certain viable cell density, where such viable cell density can be at least or about 0.01×10⁶ vc/mL, 0.1×10⁶ vc/mL, 1×10⁶ vc/mL, 2×10⁶ vc/mL, 3×10⁶ vc/mL, 4×10⁶ vc/mL, 5×10⁶ vc/mL, 6×10⁶ vc/mL, 7×10⁶ vc/mL, 8×10⁶ vc/mL, 9×10⁶ vc/mL, 10×10⁶ vc/mL, 11×10⁶ vc/mL, 12×10⁶ vc/mL, 13×10⁶ vc/mL, 14×10⁶ vc/mL, 15×10⁶ vc/mL, 16×10⁶ vc/mL, 17×10⁶ vc/mL, 18×10⁶ vc/mL, 19×10⁶ vc/mL, 20×10⁶ vc/mL, 21×10⁶ vc/mL, 22×10⁶ vc/mL, 23×10⁶ vc/mL, 24×10⁶ vc/mL, 25×10⁶ vc/mL, 26×10⁶ vc/mL, 27×10⁶ vc/mL, 28×10⁶ vc/mL, 29×10⁶ vc/mL, 30×10⁶ vc/mL, or higher, or a range of viable cell density including and between any two of the foregoing values, such as about 0.01×10⁶ vc/mL to 30×10⁶ vc/mL; 2×10⁶ to 25×10⁶ vc/mL; 5×10⁶ to 25×10⁶ vc/mL; 2×10⁶ to 30×10⁶ vc/mL; 5×10⁶ to 30×10⁶ vc/mL; 10×10⁶ to 20×10⁶ vc/mL; 11×10⁶ to 20×10⁶ vc/mL; 12×10⁶ to 20×10⁶ vc/mL; 13×10⁶ to 20×10⁶ vc/mL; 14×10⁶ to 20×10⁶ vc/mL; 15×10⁶ to 20×10⁶ vc/mL; 16×10⁶ to 20×10⁶ vc/mL; 17×10⁶ to 20×10⁶ vc/mL; 18×10⁶ to 20×10⁶ vc/mL; 19×10⁶ to 20×10⁶ vc/mL; 10×10⁶ to 21×10⁶ vc/mL; 11×10⁶ to 21×10⁶ vc/mL; 12×10⁶ to 21×10⁶ vc/mL; 13×10⁶ to 21×10⁶ vc/mL; 14×10⁶ to 21×10⁶ vc/mL; 15×10⁶ to 21×10⁶ vc/mL; 16×10⁶ to 21×10⁶ vc/mL; 17×10⁶ to 21×10⁶ vc/mL; 18×10⁶ to 21×10⁶ vc/mL; 19×10⁶ to 21×10⁶ vc/mL; 20×10⁶ to 21×10⁶ vc/mL; 10×10⁶ to 22×10⁶ vc/mL; 11×10⁶ to 22×10⁶ vc/mL; 12×10⁶ to 22×10⁶ vc/mL; 13×10⁶ to 22×10⁶ vc/mL; 14×10⁶ to 22×10⁶ vc/mL; 15×10⁶ to 22×10⁶ vc/mL; 16×10⁶ to 22×10⁶ vc/mL; 17×10⁶ to 22×10⁶ vc/mL; 18×10⁶ to 22×10⁶ vc/mL; 19×10⁶ to 22×10⁶ vc/mL; 20×10⁶ to 22×10⁶ vc/mL; 21×10⁶ to 22×10⁶ vc/mL; 10×10⁶ to 23×10⁶ vc/mL; 11×10⁶ to 23×10⁶ vc/mL; 12×10⁶ to 23×10⁶ vc/mL; 13×10⁶ to 23×10⁶ vc/mL; 14×10⁶ to 23×10⁶ vc/mL; 15×10⁶ to 23×10⁶ vc/mL; 16×10⁶ to 23×10⁶ vc/mL; 17×10⁶ to 23×10⁶ vc/mL; 18×10⁶ to 23×10⁶ vc/mL; 19×10⁶ to 23×10⁶ vc/mL; 20×10⁶ to 23×10⁶ vc/mL; 21×10⁶ to 23×10⁶ vc/mL; 10×10⁶ to 24×10⁶ vc/mL; 11×10⁶ to 24×10⁶ vc/mL; 12×10⁶ to 24×10⁶ vc/mL; 13×10⁶ to 24×10⁶ vc/mL; 14×10⁶ to 24×10⁶ vc/mL; 15×10⁶ to 24×10⁶ vc/mL; 16×10⁶ to 24×10⁶ vc/mL; 17×10⁶ to 24×10⁶ vc/mL; 18×10⁶ to 24×10⁶ vc/mL; 19×10⁶ to 24×10⁶ vc/mL; 20×10⁶ to 24×10⁶ vc/mL; 21×10⁶ to 24×10⁶ vc/mL; 22×10⁶ to 24×10⁶ vc/mL; 23×10⁶ to 24×10⁶ vc/mL; 10×10⁶ to 25×10⁶ vc/mL; 11×10⁶ to 25×10⁶ vc/mL; 12×10⁶ to 25×10⁶ vc/mL; 13×10⁶ to 25×10⁶ vc/mL; 14×10⁶ to 25×10⁶ vc/mL; 15×10⁶ to 25×10⁶ vc/mL; 16×10⁶ to 25×10⁶ vc/mL; 17×10⁶ to 25×10⁶ vc/mL; 18×10⁶ to 25×10⁶ vc/mL; 19×10⁶ to 25×10⁶ vc/mL; 20×10⁶ to 25×10⁶ vc/mL; 21×10⁶ to 25×10⁶ vc/mL; 22×10⁶ to 25×10⁶ vc/mL; 23×10⁶ to 25×10⁶ vc/mL; 24×10⁶ to 25×10⁶ vc/mL; 10×10⁶ to 26×10⁶ vc/mL; 11×10⁶ to 26×10⁶ vc/mL; 12×10⁶ to 26×10⁶ vc/mL; 13×10⁶ to 26×10⁶ vc/mL; 14×10⁶ to 26×10⁶ vc/mL; 15×10⁶ to 26×10⁶ vc/mL; 16×10⁶ to 26×10⁶ vc/mL; 17×10⁶ to 26×10⁶ vc/mL; 18×10⁶ to 26×10⁶ vc/mL; 19×10⁶ to 26×10⁶ vc/mL; 20×10⁶ to 26×10⁶ vc/mL; 21×10⁶ to 26×10⁶ vc/mL; 22×10⁶ to 26×10⁶ vc/mL; 23×10⁶ to 26×10⁶ vc/mL; 24×10⁶ to 26×10⁶ vc/mL; 25×10⁶ to 26×10⁶ vc/mL; 10×10⁶ to 27×10⁶ vc/mL; 11×10⁶ to 27×10⁶ vc/mL; 12×10⁶ to 27×10⁶ vc/mL; 13×10⁶ to 27×10⁶ vc/mL; 14×10⁶ to 27×10⁶ vc/mL; 15×10⁶ to 27×10⁶ vc/mL; 16×10⁶ to 27×10⁶ vc/mL; 17×10⁶ to 27×10⁶ vc/mL; 18×10⁶ to 27×10⁶ vc/mL; 19×10⁶ to 27×10⁶ vc/mL; 20×10⁶ to 27×10⁶ vc/mL; 21×10⁶ to 27×10⁶ vc/mL; 22×10⁶ to 27×10⁶ vc/mL; 23×10⁶ to 27×10⁶ vc/mL; 24×10⁶ to 27×10⁶ vc/mL; 25×10⁶ to 27×10⁶ vc/mL; 26×10⁶ to 27×10⁶ vc/mL; 10×10⁶ to 28×10⁶ vc/mL; 11×10⁶ to 28×10⁶ vc/mL; 12×10⁶ to 28×10⁶ vc/mL; 13×10⁶ to 28×10⁶ vc/mL; 14×10⁶ to 28×10⁶ vc/mL; 15×10⁶ to 28×10⁶ vc/mL; 16×10⁶ to 28×10⁶ vc/mL; 17×10⁶ to 28×10⁶ vc/mL; 18×10⁶ to 28×10⁶ vc/mL; 19×10⁶ to 28×10⁶ vc/mL; 20×10⁶ to 28×10⁶ vc/mL; 21×10⁶ to 28×10⁶ vc/mL; 22×10⁶ to 28×10⁶ vc/mL; 23×10⁶ to 28×10⁶ vc/mL; 24×10⁶ to 28×10⁶ vc/mL; 25×10⁶ to 28×10⁶ vc/mL; 26×10⁶ to 28×10⁶ vc/mL; 27×10⁶ to 28×10⁶ vc/mL; 10×10⁶ to 29×10⁶ vc/mL; 11×10⁶ to 29×10⁶ vc/mL; 12×10⁶ to 29×10⁶ vc/mL; 13×10⁶ to 29×10⁶ vc/mL; 14×10⁶ to 29×10⁶ vc/mL; 15×10⁶ to 29×10⁶ vc/mL; 16×10⁶ to 29×10⁶ vc/mL; 17×10⁶ to 29×10⁶ vc/mL; 18×10⁶ to 29×10⁶ vc/mL; 19×10⁶ to 29×10⁶ vc/mL; 20×10⁶ to 29×10⁶ vc/mL; 21×10⁶ to 29×10⁶ vc/mL; 22×10⁶ to 29×10⁶ vc/mL; 23×10⁶ to 29×10⁶ vc/mL; 24×10⁶ to 29×10⁶ vc/mL; 25×10⁶ to 29×10⁶ vc/mL; 26×10⁶ to 29×10⁶ vc/mL; 27×10⁶ to 29×10⁶ vc/mL; 28×10⁶ to 29×10⁶ vc/mL; 10×10⁶ to 30×10⁶ vc/mL; 11×10⁶ to 30×10⁶ vc/mL; 12×10⁶ to 30×10⁶ vc/mL; 13×10⁶ to 30×10⁶ vc/mL; 14×10⁶ to 30×10⁶ vc/mL; 15×10⁶ to 30×10⁶ vc/mL; 16×10⁶ to 30×10⁶ vc/mL; 17×10⁶ to 30×10⁶ vc/mL; 18×10⁶ to 30×10⁶ vc/mL; 19×10⁶ to 30×10⁶ vc/mL; 20×10⁶ to 30×10⁶ vc/mL; 21×10⁶ to 30×10⁶ vc/mL; 22×10⁶ to 30×10⁶ vc/mL; 23×10⁶ to 30×10⁶ vc/mL; 24×10⁶ to 30×10⁶ vc/mL; 25×10⁶ to 30×10⁶ vc/mL; 26×10⁶ to 30×10⁶ vc/mL; 27×10⁶ to 30×10⁶ vc/mL; 28×10⁶ to 30×10⁶ vc/mL; or 29×10⁶ to 30×10⁶ vc/mL, or some other range. In some embodiments, the host cells are HEK293 cells in suspension culture.

In some embodiments, host cells producing a desired biological product, such as an AAV vector, are lysed while suspended in a volume of a physiologically compatible fluid, such as the media in which they were grown, or in some embodiments the media in which they were transfected (which can be the same media as that in which they were grown), where such volume can be at least or about 10 mL, 50 mL, 100 mL, 250 mL, 500 mL, 750 mL, 1 liter (L), 2 L, 5 L, 10 L, 20 L, 25 L, 50 L, 75 L, 100 L, 150 L, 200 L, 250 L, 300 L, 400 L, 500 L, 600 L, 700 L, 750 L, 800 L, 900 L, 1000 L, 1250 L, 1500 L, 1750 L, 2000 L, 3000 L, 4000 L, 5000 L, or a greater volume, or a range including and between any of the foregoing volumes, such as 10 mL to 5 L, 10 mL to 10 L, 10 mL to 50 L, 10 mL to 100 L, 10 ml to 5000 L, 100 mL to 5 L, 100 mL to 10 L, 100 mL to 50 L, 100 mL to 100 L, 1 L to 5 L, 1 L to 10 L, 1 L to 50 L, 1 L to 100 L, 2 L to 5 L, 2 L to 10 L, 2 L to 50 L, 2 L to 100 L, 5 L to 10 L, 5 L to 50 L, 5 L to 100 L, 10 L to 50 L, 10 L to 100 L, 50 L to 250 L, 50 L to 500 L, 100 L to 250 L, 100 L to 500 L, 100 L to 1000 L, 250 L to 500 L, 250 L to 1000 L, 250 L to 2000 L, 500 L to 1000 L, 500 L to 2000 L, 500 L to 5000 L, or some other range. In some embodiments, the volume is that of a sample of the host cells, which sample can comprise the entire volume of a suspension cell culture in a bioreactor, for example. In some embodiments, the host cells are HEK293 cells in suspension culture. In other embodiments, before lysis, host cells can be concentrated into a volume of physiologically compatible fluid that is smaller compared to the volume of media in which they were grown.

The final concentration of a detergent such as CTAB, a modifier such as MgSO₄, or both, added to a suspension of host cells, to a mixture thereof or to a culture of adherent cells can be any concentration effective to lyse the host cells and precipitate the host cell DNA while not damaging or denaturing a desired biological product, such as an AAV vector, released from the cells after lysis. Concentrations can be expressed in terms of percentage or molarity, and if expressed as a percentage, can be calculated as % weight by volume or % weight by weight, the values of which will not be significantly different for dilute aqueous solutions.

In some embodiments, a final concentration of a detergent (e.g., a quaternary amine compound) can be at least or about 0.005%, 0.0075%, 0.01%, 0.015%, 0.02%, 0.025%, 0.03%, 0.035%, 0.04%, 0.045%, 0.05%, 0.055%, 0.06%, 0.065%, 0.07%, 0.075%, 0.08%, 0.085%, 0.09%, 0.095%, 0.10%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1.00%, 2.00%, 3.00%, 4.00%, or 5.00% (w/v) or a range of concentrations including and between any two of the foregoing values, such as from about 0.005% to 5.00%, 0.005% to 1.0%, 0.01% to 5.0%, 0.01% to 1.0%, 0.01% to 0.075%, 0.02% to 0.06%, 0.045% to 0.065%, 0.05% to 5.00%, 0.10% to 2.50%, 0.20% to 1.25%, 0.20% to 0.75%, 0.25% to 0.75%, 0.25% to 0.65%, 0.20% to 0.70%, 0.30% to 0.70%, 0.35% to 0.65%, 0.40% to 0.60%, or 0.45% to 0.55% (w/v), or some other range. In some embodiments, the final concentration of a detergent (e.g., a quaternary amine compound) can be at least or about 0.01% to 0.1% (w/v), optionally 0.05% (w/v). In some embodiments, the final concentration of a detergent can be at least or about 0.1% to 1.0% (w/v), optionally 0.5% (w/v).

In some embodiments, a detergent is a quaternary amine detergent, for example CTAB, which can be used to lyse host cells and precipitate host cell DNA at a final concentration of at least or about 0.005%, 0.0075%, 0.01%, 0.015%, 0.02%, 0.025%, 0.03%, 0.035%, 0.04%, 0.045%, 0.05%, 0.055%, 0.06%, 0.065%, 0.07%, 0.075%, 0.08%, 0.085%, 0.09%, 0.095%, 0.10%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1.00%, 2.00%, 3.00%, 4.00%, or 5.00% (w/v), or a range of concentrations including and between any two of the foregoing values, such as from about 0.005% to 5.00%, 0.005% to 1.0%, 0.01% to 5.0%, 0.01% to 1.0%, 0.01% to 0.075%, 0.02% to 0.06%, 0.045% to 0.065%, 0.05% to 5.00%, 0.10% to 2.50%, 0.20% to 1.25%, 0.20% to 0.75%, 0.25% to 0.75%, 0.25% to 0.65%, 0.20% to 0.70%, 0.30% to 0.70%, 0.35% to 0.65%, 0.40% to 0.60%, or 0.45% to 0.55% (w/v), or some other range. In some embodiments, the final concentration of a quaternary amine compound (e.g., CTAB) can be at least or about 0.01% to 0.1% (w/v), optionally 0.05% (w/v). In some embodiments, the final concentration of a quaternary amine compound (e.g., CTAB) can be at least or about 0.1% to 1.0% (w/v), optionally 0.5% (w/v).

In some embodiments, a concentration of a modifier (e.g., a salt) can be at least or about 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 110 mM, 120 mM, 130 mM, 140 mM, 150 mM, 160 mM, 170 mM, 180 mM, 190 mM, 200 mM, 225 mM, 250 mM, 275 mM, 300 mM, 325 mM, 350 mM, 375 mM, 400 mM, 425 mM, 450 mM, 475 mM, 500 mM, 600 mM, 700 mM, 800 mM, 900 mM, 1 M or a range of concentrations including and between any two of the foregoing values, such as from about 1 mM to 500 mM, 10 mM to 400 mM, 25 mM to 300 mM, 25 mM to 200 mM, 40 mM to 300 mM, 40 mM to 100 mM, 40 mM to 60 mM, 45 mM to 55 mM, 50 mM to 75 mM, 50 mM to 100 mM, 50 mM to 150 mM or 50 mM to 60 mM or some other range. In some embodiments, the final concentration of a modifier (e.g., a salt) can be at least or about 25 mM to 75 mM or about 40 mM to 60 mM, optionally 50 mM.

In some embodiments, a modifier is a salt, for example MgSO₄, which can be used with a detergent to lyse host cells and precipitate host cell DNA at a final concentration of at least or about 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 110 mM, 120 mM, 130 mM, 140 mM, 150 mM, 160 mM, 170 mM, 180 mM, 190 mM, 200 mM, 225 mM, 250 mM, 275 mM, 300 mM, 325 mM, 350 mM, 375 mM, 400 mM, 425 mM, 450 mM, 475 mM, 500 mM, 600 mM, 700 mM, 800 mM or 900 mM or a range of concentrations including and between any two of the foregoing values, such as from about 1 mM to 500 mM, 10 mM to 400 mM, 25 mM to 300 mM, 25 mM to 200 mM, 40 mM to 300 mM, 40 mM to 100 mM, 40 mM to 60 mM, 45 mM to 55 mM, 50 mM to 75 mM, 50 mM to 100 mM, 50 mM to 150 mM or 50 mM to 60 mM or some other range. In some embodiments, the final concentration of a salt (e.g., a MgSO₄) can be at least or about 25 mM to 75 mM or about 40 mM to 60 mM, optionally 50 mM.

In some embodiments, a sample of host cells suspended in a physiologically compatible fluid is lysed by adding to the fluid a solution comprising CTAB, wherein the final concentration of CTAB in the mixture is about at least or about 0.01% to 0.1% (w/v), optionally 0.05% (w/v). In some embodiments, a sample of host cells suspended in a physiologically compatible fluid is lysed by adding to the fluid a solution comprising CTAB, wherein the final concentration of CTAB in the mixture is about at least or about 0.01% to 0.1% (w/v), optionally 0.05% (w/v), and a solution comprising MgSO₄, wherein the final concentration of MgSO₄ in the mixture is about 25 mM to 75 mM, optionally 50 mM. In some embodiments, host cell DNA released from the lysed cells is precipitated to produce and flocculant and a supernatant. In some embodiments, the host cells are HEK293 cells in suspension culture. In some embodiments, the host cells produce a AAV vector. In some embodiments, the host cells produce a rAAV3B vector.

In some embodiments, a sample of host cells suspended in a physiologically compatible fluid comprising MgSO₄ (optionally at a concentration of about 25 mM to 75 mM) is lysed by adding to the fluid a solution comprising CTAB, wherein the final concentration of CTAB in the mixture is about at least or about 0.01% to 0.1% (w/v), optionally 0.05% (w/v). In some embodiments, host cell DNA released from the lysed cells is precipitated to produce and flocculant and a supernatant. In some embodiments, the host cells are HEK293 cells in suspension culture. In some embodiments, the host cells produce a AAV vector. In some embodiments, the host cells produce a rAAV3B vector.

In some embodiments, a sample of host cells suspended in a physiologically compatible fluid is lysed by adding to the fluid a solution comprising CTAB, wherein the final concentration of CTAB in the mixture is about at least or about 0.1% to 1.0% (w/v), optionally 0.5% (w/v). In some embodiments, a sample of host cells suspended in a physiologically compatible fluid is lysed by adding to the fluid a solution comprising CTAB, wherein the final concentration of CTAB in the mixture is about at least or about 0.1% to 1.0% (w/v), optionally 0.5% (w/v), and a solution comprising MgSO₄, wherein the final concentration of MgSO₄ in the mixture is about 25 mM to 75 mM, optionally 50 mM. In some embodiments, host cell DNA released from the lysed cells is precipitated to produce and flocculant and a supernatant. In some embodiments, the host cells are HEK293 cells in suspension culture. In some embodiments, the host cells produce a AAV vector. In some embodiments, the host cells produce a rAAV3B vector.

In some embodiments, a sample of host cells suspended in a physiologically compatible fluid comprising MgSO₄ (optionally at a concentration of about 25 mM to 75 mM) is lysed by adding to the fluid a solution comprising CTAB, wherein the final concentration of CTAB in the mixture is about at least or about 0.1% to 1.0% (w/v), optionally 0.5% (w/v). In some embodiments, host cell DNA released from the lysed cells is precipitated to produce and flocculant and a supernatant. In some embodiments, the host cells are HEK293 cells in suspension culture. In some embodiments, the host cells produce a AAV vector. In some embodiments, the host cells produce a rAAV3B vector.

In some embodiments, CTAB at a final concentration of about 0.05% and MgSO₄ at a final concentration of about 50 mM is effective to lyse host cells even at high viable cell densities at the time of lysis (harvest), such as at least or about 9×10⁶ vc/mL, 10×10⁶ vc/mL, 11×10⁶ vc/mL, 12×10⁶ vc/mL, 13×10⁶ vc/mL, 14×10⁶ vc/mL, 15×10⁶ vc/mL, 16×10⁶ vc/mL, 17×10⁶ vc/mL, 18×10⁶ vc/mL, 19×10⁶ vc/mL, 20×10⁶ vc/mL, 21×10⁶ vc/mL, 22×10⁶ vc/mL, 23×10⁶ vc/mL, 24×10⁶ vc/mL, 25×10⁶ vc/mL, 26×10⁶ vc/mL, 27×10⁶ vc/mL, 28×10⁶ vc/mL, 29×10⁶ vc/mL, 30×10⁶ vc/mL, or a range of viable cell density including and between any two of the foregoing values, such as from about 9×10⁶ vc/mL to 15×10⁶ vc/mL, 10×10⁶ vc/mL to 14×10⁶ vc/mL, 18×10⁶ vc/mL to 24×10⁶ vc/mL, 10×10⁶ vc/mL to 30×10⁶ vc/mL, or 15×10⁶ vc/mL to 25×10⁶ vc/mL. In the latter two exemplary ranges, a final concentration of 0.05% CTAB is equivalent to 0.005% to 0.0017% CTAB per 10⁶ viable host cells per mL, or to 0.0033% to 0.002% CTAB per 10⁶ viable host cells per mL, respectively. In the latter two exemplary ranges, a final concentration of 50 mM MgSO₄ is equivalent to 5.0 mM to 1.7 mM MgSO₄ per 10⁶ viable host cells per mL, or to 3.3 mM to 2.0 mM MgSO₄ per 10⁶ viable host cells per mL, respectively. In some non-limiting embodiments, the host cells are HEK293 cells grown in suspension culture which produce an AAV vector. In some embodiments, an AAV vector is a rAAV3B vector.

In some embodiments, host cells are suspended in a physiologically compatible fluid and a solution comprising CTAB, a solution comprising MgSO₄ or both are mixed for a period of time during the addition of the solution comprising CTAB, the solution comprising MgSO₄ or both, and/or after all of the solution comprising CTAB, the solution comprising MgSO₄ or both has been added, to effect thorough mixing of the two or more solutions. In some embodiments, such mixing can be performed for at least or about 1 min, about 5 mins, 10 mins, 15 mins, 20 mins, 30 mins, 40 mins, 50 mins, 60 mins, 70 mins, 75 mins, 80 mins, 90 mins, 100 mins, 115 mins, 120 mins, or more, or a range including and between any two of the foregoing times, such as 5 mins to 30 min, such as 15 mins to 90 mins, such as 20 mins to 40 mins, or some other range of time.

In some embodiments, the host cells are suspended in a physiologically compatible fluid and a solution comprising MgSO₄ is mixed for at least 5 minutes during the addition of the solution comprising MgSO₄ and/or after all of the solution comprising MgSO₄ has been added, to effect thorough mixing of the two solutions.

In some embodiments, the host cells are suspended in a physiologically compatible fluid and a solution comprising CTAB is mixed for at least 5 minutes during the addition of the solution comprising CTAB and/or after all of the solution comprising CTAB has been added, to effect thorough mixing of the two solutions.

In some embodiments, the host cells are suspended in a physiologically compatible fluid comprising MgSO₄ (optionally at a concentration of about 25 mM to 75 mM) and a solution comprising CTAB is mixed for at least 5 minutes during the addition of the solution comprising CTAB and/or after all of the solution comprising CTAB has been added, to effect thorough mixing of the two solutions.

In some embodiments, after mixing, the mixture of the host cells suspended in the physiologically compatible fluid and the solution comprising CTAB is held, or incubated, for a period of time without active mixing. In some embodiments, the hold period can be at least or about 1 min, about 5 mins, 10 mins, 15 mins, 20 mins, 30 mins, 40 mins, 50 mins, 60 mins, 70 mins, 75 mins, 80 mins, 90 mins, 100 mins, 115 mins, 120 mins, 150 mins, 3 hrs, 4 hrs, 5 hrs, 6 hrs, or more, or a range including and between any two of the foregoing times, such as 5 min to 30 min, such as 15 mins to 90 mins, or some other range of time. In some embodiments, the mixing and/or holding are performed at about room temperature, for example, 20 to 22° C., or some other temperature, such as about 2° C. to 8° C., 4° C., or 37° C.

In some embodiments, a method of preparing a cell lysate comprises lysing host cells and precipitating DNA released from the host cells to produce a lysate comprising a flocculant and a supernatant. In some embodiments, the host cells are suspended in a physiologically compatible fluid and are lysed chemically by adding to the fluid a solution comprising a detergent in a concentration sufficient to cause cell lysis and flocculation of host cell DNA. In some embodiments, the detergent is CTAB, optionally at a concentration of about 0.01% to 0.1% (w/v) or about 0.05% (w/v). In some embodiments, the method further comprises adding to the fluid a solution comprising a modifier. In some embodiments, the modifier is MgSO₄, optionally at a concentration of about 25 mM to 75 mM or about 50 mM. In some embodiments, the solution comprising the modifier is added to the fluid before the solution comprising the detergent is added to the fluid. In some embodiments, the solution comprising the modifier is added to the fluid followed by a mixing for at least 5 minutes (e.g., 6 minutes, 7 minutes, 8 minutes, 9 minutes, 10 minutes, 15 minutes, 20 minutes, 25 minutes, 30 minutes, 35 minutes, 40 minutes, 45 minutes, 50 minutes, 55 minutes, 60 minutes, 1.5 hours, 2 hours) to produce a mixture. In some embodiments, the solution comprising the detergent is added to the mixture followed by mixing for at least 5 minutes to produce the flocculant and the supernatant. In some embodiments, the physiologically compatible fluid, the solution comprising the detergent and the solution comprising the modifier are mixed by rocking or stirring for 5 minutes to 60 minutes, optionally for 30 minutes at room temperature of 18° C. to 25° C. In some embodiments, the method further comprises filtration of the supernatant and flocculant to produce a clarified lysate. In some embodiments, the clarified lysate comprises rAAV vectors, optionally rAAV3B vectors.

Separating Flocculated Host Cell DNA from Supernatant

Flocculated host cell DNA can be separated from a supernatant by any technique known in the art to be effective to separate flocculated host cell DNA and a supernatant. In some embodiments, flocculated host cell DNA can be separated from a supernatant by allowing flocs to settle under the influence of gravity for a period of time to the bottom of a container in which host cell lysate and DNA precipitation solution were mixed, usually without mixing while settling is occurring. Alternatively, the flocculated host cell DNA can be separated from the supernatant by centrifugation, such as by continuous flow centrifugation. Flocs can also be removed from the mixture through one or more depth filters.

In some embodiments, a supernatant is filtered to remove a flocculant for example by filtering through one or more depth filters and/or membrane filters. Filtering a supernatant produces in a clarified lysate (may also be referred to as a clarified supernatant or a clarified harvest). In some embodiments, one or more of the filters has a nominal retention rating, or average pore size, of less than or equal to about 100 μm, 50 μm, 40 μm, 30 μm, 25 μm, 20 μm, 15 μm, 10 μm, 5 μm, 2 μm, 1 μm, or 0.5 μm.

In some embodiments, a partially clarified supernatant that has formed above a layer of flocculated host cell DNA that has been allowed to settle under the influence of gravity may be removed by pumping. For example, the supernatant can be pumped out through a tube inserted from above, the end of which is immersed in the supernatant but positioned above the flocculant layer, or through a port inserted through a wall of the container located above the layer of flocculant. Alternatively, the flocculant can be pumped out through a tube inserted from above, the end of which is immersed in the flocculant, or through a port at the bottom of the container or inserted through a wall of the container located below the supernatant. A combination of these methods can also be used. Typically, after being removed or separated from the flocculated host cell DNA, the supernatant is transferred to a new container.

In some embodiments, a partially clarified supernatant, after having been removed or separated from the flocculated host cell DNA, such as by pumping, is filtered to remove any flocs that may have been carried along during the removal process, for example by filtering through one or more depth filters and/or membrane filters. Filtering the partially clarified supernatant (lysate) results in a clarified supernatant (lysate). In some embodiments, one or more of the filters has a nominal retention rating, or average pore size, of less than or equal to about 100 μm, 50 μm, 40 μm, 30 μm, 25 μm, 20 μm, 15 μm, 10 μm, 5 μm, 2 μm, 1 μm, or 0.5 μm.

### Downstream Purification Steps

As used herein, the terms “purify,” “purified,” “purification,” and the like, when used in connection with a biological product, or sample or preparation thereof, indicate a relative increase or improvement in purity compared with a starting material from which the biological product is derived, and/or a prior intermediate purification step in some scheme of sequential purification steps intended to purify the biological product, and does not require a particular qualitative or quantitative degree of purity, unless otherwise specified.

In some embodiments, after separating a flocculant and a supernatant by filtration to produce a clarified lysate, a biological product in the lysate can be further purified in at least one downstream processing step known in the art to be effective to purify such biological product. For example, if the product is a monoclonal antibody, then the product could be purified by pumping the mixture through an affinity chromatography column in which the resin or matrix used to fill the column contains protein A. In other embodiments, depending on the nature of the biological product, the downstream processing step can comprise precipitation in a lyotropic salt, such as ammonium sulfate. In other embodiments the downstream processing step can comprise performing at least one type of chromatography. Many types of chromatography useful in the methods of the disclosure are known in the art including, without limitation, size exclusion chromatography (SEC); affinity chromatography, using any affinity ligand attached to the chromatography resin or matrix capable of specific binding to the biological product, such as an antibody, or antigen binding fragment thereof, lectin, protein A, protein G, protein L, or glycan, etc.; immobilized metal chelate chromatography (IMAC); thiophilic adsorption chromatography; hydrophobic interaction chromatography (HIC); multimodal chromatography (MMC); pseudo-affinity chromatography; and ion exchange chromatography (IEX or IEC), such as anion exchange chromatography (AEX) or cation exchange chromatography (CEX). In other embodiments, the downstream processing step can comprise desalting or buffer exchange, filtering, such as ultrafiltration, nanofiltration, and/or diafiltration, or concentrating the biological product, for example using tangential flow filtration (TFF). Use of more than one downstream processing step is possible, and the plurality of downstream processing steps can be performed in any order according to the knowledge of those ordinarily skilled in the art.

In some embodiments, the biological product is an AAV vector, and the downstream step useful for further purifying the vector can comprise, without limitation, performing at least one chromatography step. In some embodiments, the chromatography step comprises antibody-based affinity ligand purification in which an antibody, or antibody fragment thereof, is attached to a stationary phase matrix or resin loaded into a chromatography column which is then equilibrated with a suitable buffer, followed by pumping the supernatant and salt solution mixture containing the vector through the column, and then eluting the vector that specifically bound to the ligand. In some embodiments, the antibody bound to the solid phase can be an IgG, or fragment thereof, or a single-chain camelid antibody (such as a heavy chain variable region camelid antibody). Non-limiting examples of such resins include Sepharose AVB, POROS CaptureSelect AAVX, POROS CaptureSelect AAV8, and POROS CaptureSelect AAV9. See, e.g., Terova, O, et al., Affinity Chromatography Accelerates Viral Vector Purification for Gene Therapies, BioPharm Intl. eBook pp. 27-35 (2017); Mietzsch, M, et al., Characterization of AAV-Specific Affinity Ligands: Consequences for Vector Purification and Development Strategies, Mol. Ther. Meth. & Clin. Dev., 19:362-73 (2020); Rieser, R, et al., Comparison of Different Liquid Chromatography-Based Purification Strategies for Adeno-Associated Virus Vectors, Pharmaceutics 13, 748 (2021) (doi.org/10.3390/pharmaceutics13050748).

In other embodiments, the chromatography step comprises use of a stationary phase to which is bound the same type of ligand that certain AAV serotypes use in binding to cells, such as a glycan, such as sialic acid (e.g., an O-linked or N-linked sialic acid), galactose, heparin, or heparan sulfate, or a proteoglycan, such as a heparan or heparin sulfate proteoglycan (HSPG). For example, an affinity matrix containing sialic acid residues can be used to purify AAV vectors with capsids that specifically bind to sialic acid (e.g., AAV1, AAV4, AAV5, or AAV6); an affinity matrix containing galactose can be used to purify AAV vectors with capsids that specifically bind to galactose (e.g., AAV9); and an affinity matrix containing heparin, heparan, or HSPG can be used to purify AAV vectors with capsids that specifically bind to HSPG (e.g., AAV2, AAV3 (including AAV3A and AAV3B), AAV6, AAV13, AAV-DJ). In yet other exemplary non-limiting embodiments, depending on the physicochemical characteristics of the vector, such as the charge on the capsid, AAV vectors can be further purified by performing anion exchange, cation exchange, or hydrophobic interaction chromatography. Any other downstream process step useful for purifying AAV vectors known in the art may be used as well.

In some embodiments, the methods of the disclosure are effective to improve the performance of at least one downstream processing step. In some embodiments, the downstream processing step is affinity chromatography and improved performance is measured as a number of purification cycles before yield of a biological product, for example, an AAV vector, falls below a certain threshold, such as less than 80%, 70%, 60%, or 50%. As known in the art, chromatography is typically performed by packing a chromatography column of any suitable size with fresh, unused stationary phase resin or matrix suitable for the type of chromatography being performed, such as an affinity resin, and then washing and/or equilibrating the resin with suitable equilibration solution(s) in preparation for loading the column with the sample to be purified. The column is then ready for the first purification cycle in which a liquid sample containing the biological product to be purified is loaded onto and pumped through the column. Once the entire sample has been pumped through, the column may be washed with any suitable non-denaturing wash solution(s) to remove contaminants while the biological product is retained, usually non-covalently, on the stationary phase. The product can thereafter be eluted by pumping through the column any suitable elution solution and collecting the eluate, which may be collected in elution fractions which are thereafter tested to determine the amount of biological product in each, after which fractions containing a significant amount of the biological product can be pooled. In some embodiments, after elution, the stationary phase can be cleaned in place with any suitable clean in place solution(s) containing chemicals, such as acids, bases or chaotropic salts, to remove any residual product or contaminants, and then re-equilibrated with the equilibration solution in preparation for a subsequent purification cycle. Thus, as used herein, a purification cycle comprises running a sample through a chromatography column and then eluting the desired biological product, such as an AAV vector, retained on the stationary phase.

In the context of a downstream process step involving chromatography, “yield” of a biological product means the total amount of the product in the eluate pool expressed as a percentage of the total amount of the product in a sample before the chromatography step. The amount of the biological product can be determined using any method known in the art. For example, if the product is an AAV vector, the amount of the vector can be quantified using quantitative PCR (pPCR) using primers against the ITRs, or sequences in the transgene or other parts of the expression cassette, or using digital droplet PCR (ddPCR), and expressed as a titer in terms of vector genomes per unit volume, such as milliliters (vg/mL). See, e.g., Dobnik, D, et al., Accurate Quantification and Characterization of Adeno-Associated Viral Vectors, Front. Microbiol., Vol. 10, Art. 1570, pp. 1-13 (2019); Wang, Y, et al., A qPCR Method for AAV Genome Titer with ddPCR-Level of Accuracy and Precision, Mol. Ther.: Methods & Clin. Devel., 19:341-6 (2020); Werling, N J, et al., Systematic Comparison and Validation of Quantitative Real-Time PCR Methods for the Quantitation of Adeno-Associated Viral Product, Hum. Gene Ther. Meth. 26:82-92 (2015).

In some embodiments, the methods of the disclosure are effective to purify an AAV vector by performing affinity chromatography, where the number of affinity chromatography purification cycles that can be performed before yield of an AAV vector falls below 80%, 70%, 60%, or 50% is at least 5, 6, 7, 8, 9, 10, 11, 12, 13, 14, 15, or more cycles. In some of these embodiments, the chromatography is immunoaffinity chromatography, and the vector contains a capsid that binds more strongly to sialic acid or galactose than to HSPG, or does not bind specifically, or only weakly binds to HSPG, for example, AAV1, AAV4, AAV5, or AAV9. In some of these embodiments, the chromatography is immunoaffinity chromatography, and the vector contains a capsid that binds more strongly to HSPG, for example AAV2, AAV3 (including AAV3A and AAV3B), AAV6, AAV13, and AAV-DJ. In other embodiments, the methods of the disclosure are effective to purify an AAV vector by performing affinity chromatography, where the number of affinity chromatography purification cycles that can be performed before yield of an AAV vector falls below 50% is at least 5, 6, 7, 8, 9, 10, 11, 12, 13, 14, 15, or more cycles. In some of these embodiments, the chromatography is immunoaffinity chromatography, and the vector contains a capsid that binds more strongly to sialic acid or galactose than to HSPG, for example, AAV1, AAV4, AAV5, AAV6, or AAV9. In some of these embodiments, the chromatography is immunoaffinity chromatography, and the vector contains a capsid that binds more strongly to HSPG, for example AAV2, AAV3 (including AAV3A and AAV3B), AAV6, AAV13, and AAV-DJ. In other embodiments, the methods of the disclosure are effective to purify an AAV vector by performing affinity chromatography, where the number of affinity chromatography purification cycles that can be performed before yield of an AAV vector falls below 60% is at least 5, 6, 7, 8, 9, 10, 11, 12, 13, 14, 15, or more cycles. In some of these embodiments, the chromatography is immunoaffinity chromatography, and the vector contains a capsid that binds more strongly to sialic acid or galactose than to HSPG, or does not specifically bind or weakly binds to HSPG, for example, AAV1, AAV4, AAV5, or AAV9. In some of these embodiments, the chromatography is immunoaffinity chromatography, and the vector contains a capsid that binds more strongly to HSPG, for example AAV2, AAV3 (including AAV3A and AAV3B), AAV6, AAV13, and AAV-DJ. In other embodiments, the methods of the disclosure are effective to purify an AAV vector by performing affinity chromatography, where the number of affinity chromatography purification cycles that can be performed before yield of an AAV vector falls below 70% is at least 5, 6, 7, 8, 9, 10, 11, 12, 13, 14, 15, or more cycles. In some of these embodiments, the chromatography is immunoaffinity chromatography, and the vector contains a capsid that binds more strongly to sialic acid or galactose than to HSPG, or does not specifically bind or weakly binds to HSPG, for example, AAV1, AAV4, AAV5, or AAV9. In some of these embodiments, the chromatography is immunoaffinity chromatography, and the vector contains a capsid that binds more strongly to HSPG, for example AAV2, AAV3 (including AAV3A and AAV3B), AAV6, AAV13, and AAV-DJ.

In some embodiments, the methods of the disclosure are effective to achieve a high yield and/or purity of an active ingredient, such as a desired biological product, such as an AAV vector, in drug substance or drug product. As used in this context, “yield” means the amount of an active ingredient in drug substance compared to the amount of the same compound or substance, or a precursor thereof, in a starting material used in the synthesis or production of the active ingredient. As used herein, “drug substance” means a preparation comprising a substantially purified active ingredient resulting from a complete process intended to purify the active ingredient where the process is complete if, as designed or used in practice, it does not include or require any further steps intended to remove contaminants or further purify the active ingredient. For clarity, such further steps would not include buffer exchange, volume reduction, or addition of excipients, or like, which are merely intended to prepare drug product from drug substance, where “drug product” is the finished dosage form of the active ingredient as it would be marketed or used for administration to patients. As used herein, an “active ingredient” is any compound or substance, including a biologically derived substance, intended to furnish pharmacological activity or other direct effect in the diagnosis, cure, mitigation, treatment, or prevention of disease, or to affect the structure or any function of the human body. Non-limiting examples of active ingredients include viruses, vaccines, and virally derived vectors, such as AAV vectors and lentiviral vectors, and the like.

Purity of AAV vectors in a sample or preparation of such vectors can be determined and expressed in a variety of ways known in the art. For example, vector preparations can be analyzed on denaturing polyacrylamide gels and silver stained to detect proportions of the different viral proteins, VP1, VP2, and VP3, relative to contaminating cellular proteins. Different techniques can also be used to detect the proportion of full compared to empty capsids, with a greater percentage of full capsids indicating higher purity. As used herein, a “full capsid” is one that is concluded to contain a vector genome, and an “empty capsid” is a one that is concluded to contain either no or little nucleic acid. For example, capsids in vector preparations can be visualized using transmission electron microscopy, including cryoEM, and the numbers of full and empty capsids counted manually or using computerized image recognition algorithms. Even greater resolution can be achieved using analytical ultracentrifugation, which can discriminate between full, partially full and empty capsids. A convenient method for estimating AAV vector purity in terms of amount of contaminating empty capsids is to measure the UV light absorbance of a vector preparation at 260 nm and 280 nm, deriving the A260/A280 ratio. By calculating the theoretical extinction coefficients for a particular vector's capsid and genome, the relative concentrations of its capsid and genome in a preparation can be calculated from the A260/A280 ratio, with higher A260/A280 values indicating a greater proportion of full capsids. Additional information about methods for testing vector purity are described in Burnham B, et al., Analytical ultracentrifugation as an approach to characterize recombinant adeno-associated viral vectors, Hum. Gene Ther. Meth., 26(6):228-242 (2015); Subramanian, S, et al., Filling Adeno-Associated Virus Capsids: Estimating Success by Cryo-Electron Microscopy, Hum. Gene Ther., 30(12):1449-60 (2019); McIntosh, N L, et al., Comprehensive characterization and quantification of adeno associated vectors by size exclusion chromatography and multi angle light scattering, Nat. Sci. Reports, 11:3012, pp. 1-12 (2021); Sommer, J M, et al., Quantification of Adeno-Associated Virus Particles and Empty Capsids by Optical Density Measurement, Mol. Ther., 7(1):122-8 (2003); Wu, D, et al., Rapid Characterization of AAV gene therapy vectors by Mass Photometry, bioRxiv 2021.02.18.431916 (doi.org/10.1101/2021.02.18.431916).

In some embodiments, the methods of the disclosure are effective to achieve an acceptably low burden of host cell DNA in drug substance or drug product containing a desired biological product, such as an AAV vector, produced by host cells. The amount of host cell DNA in a sample or preparation, such as a detergent lysate of such cells, or a composition comprising biological product (such as drug substance or drug product) purified from such cells, such as an AAV vector, can be determined in any way known in the art. For example, sensitive qPCR assays have been developed designed to specifically detect repetitive sequence elements unique to the human genome (e.g., Alu repeats), and that of other species whose host cells are commonly used in manufacturing. See, e.g., Zhang, W, et al., Development and qualification of a high sensitivity, high throughput Q-PCR assay for quantitation of residual host cell DNA in purification process intermediate and drug substance samples, J. Pharma Biomed Anal 100:145-9 (2014); Wang, Y, et al., A Digestion-free Method for Quantification of Residual Host Cell DNA in rAAV Gene Therapy Products, Mol. Ther. 13:526-31 (2019). The amount of host cell DNA can be quantified and expressed as an absolute amount, such as mass in picograms (pg) or nanograms (ng), etc., in a volume, such as milliliter, or other unit, such as dose. Amounts of host cell DNA can also be normalized relative to another variable, such as the amount of AAV vector in a sample, which in some embodiments can be quantified and expressed as the number of vector genomes per milliliter, dose, etc.

What constitutes an acceptably low burden of host cell DNA in drug substance or drug product will be apparent to those of ordinary skill in the art and may depend on the type of biological product in question, as well as expectations of industry, patients, and/or regulatory authorities, such as the US FDA or the EMA, which may change with time. See, e.g., Gombold, J, et al., Lot Release and Characterization Testing of Live-Virus-Based Vaccines and Gene Therapy Products, Part 2, Bioprocess Intl. 4:46-56 (2006); Wright, J F, Product-Related Impurities in Clinical-Grade Recombinant AAV Vectors: Characterization and Risk Assessment, Biomedicines 2(1):80-97 (2014); Wang, X, et al., Residual DNA Analysis in Biologics Development: Review of Measurement and Quantitation Technologies and Future Directions, Biotech. Bioeng. 109(2):307-17 (2012).

In some embodiments, the methods of the disclosure are effective to achieve a high yield and/or purity of an AAV vector and an acceptably low burden of host cell DNA in drug substance or drug product. In some embodiments, the yield of AAV vector produced using methods of the disclosure (including as well, in some embodiments, additional downstream purification steps) can be at least or about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, or 80% or more, or any percentage yield between and including any of the foregoing values. In some embodiments, the purity of AAV vector produced using methods of the disclosure (including as well, in some embodiments, additional downstream purification steps) can be expressed as the ratio of the UV absorbance measured at 260 nm and 280 nm (i.e., A₂₆₀/A₂₃₀) which, in some embodiments, can be at least or about 0.4, 0.5, 0.6, 0.7, 0.8, 0.9, 1.0, 1.1, 1.2, 1.3, 1.4, 1.5, 1.6, 1.7, or 1.8, or more, or an A₂₆₀/A₂₃₀ between and including any of the foregoing values. In some embodiments, the purity of AAV vector produced using methods of the disclosure (including as well, in some embodiments, additional downstream purification steps) can be expressed as the percentage of full capsids in a vector preparation which, in some embodiments, can be at least or about 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, or any percentage of full capsids between and including any of the foregoing values.

In some embodiments, an acceptably low burden of host cell DNA in drug substance or drug product is one that is less than or about 100 ng, 90 ng, 80 ng, 70 ng, 60 ng, 50 ng, 40 ng, 30 ng, 20 ng, 10 ng, 5 ng, 2 ng, or 1 ng per dose, or less, or any value between and including any of the foregoing values. In other embodiments, an acceptably low burden of host cell DNA in drug substance or drug product is one that is less than or about 50 ng, 40 ng, 30 ng, 20 ng, 10 ng, 5 ng, 2 ng, 1 ng, 0.9 ng, 0.8 ng, 0.7 ng, 0.6 ng, 0.5 ng, 0.4 ng, 0.3 ng, 0.2 ng, 0.1 ng, 0.09 ng, 0.08 ng, 0.07 ng, 0.06 ng, 0.05 ng, 0.04 ng, 0.03 ng, 0.02 ng, 0.01 ng, 0.009 ng, 0.008 ng, 0.007 ng, 0.006 ng, 0.005 ng, 0.004 ng, 0.003 ng, 0.002 ng, 0.001 ng per milliliter drug substance or drug product, or less, or any value between and including any of the foregoing values. In yet other embodiments, an acceptably low burden of host cell DNA in drug substance or drug product containing an AAV vector is one that is less than or about 1000, 500, 250, 200, 150, 100, 90, 80, 70, 60, 50, 45, 40, 35, 30, 25, 20, 15, 10, 5, 2, or 1 picograms per 1×10⁹ vector genomes (pg/1×10⁹ vg), or less, or any value between and including any of the foregoing values.

Other objects, features and advantages of the present invention will be apparent from the foregoing detailed description. It should be understood, however, that the detailed description and the specific examples that follow, while indicating specific embodiments of the invention, are given by way of illustration only, since various changes, modifications and equivalents within the spirit and scope of the invention will be apparent from the detailed description and examples to those of ordinary skill in the art, and fall within the scope of the appended claims.

Unless otherwise indicated, use of the term “or” in reference to one or more members of a set of embodiments is equivalent in meaning to “and/or,” and does not require that they be mutually exclusive of each other. Unless otherwise indicated, a plurality of expressly recited numeric ranges also describes a range the lower bound of which is derived from the lower or upper bound of any one of the expressly recited ranges, and the upper bound of which is derived from the lower or upper bound of any other of the expressly recited ranges. Thus, for example, the series of expressly recited ranges “10-20, 20-30, 30-40, 40-50, 100-150, 200-250, 275-300,” also describes the ranges 10-50, 50-100, 100-200, and 150-250, among many others. Unless otherwise indicated, use of the term “about” before a series of numerical values or ranges is intended to modify not only the value or range appearing immediately after it but also each and every value or range appearing thereafter in the same series. Thus, for example, the phrase “about 1, 2, or 3,” is equivalent to “about 1, about 2, or about 3.”

All publications and references, including but not limited to articles, abstracts, patents, patent applications (whether published or unpublished), and biological sequences (including, but not limited to those identified by specific database reference numbers) cited herein are hereby incorporated herein by reference in their entirety for all purposes to the same extent as if each individual publication or reference were specifically and individually indicated to be so incorporated by reference. Any patent application to which this application claims priority directly or indirectly is also incorporated herein by reference in its entirety.

Unless otherwise indicated, the examples below describe experiments that were or are performed using standard techniques well known and routine to those of ordinary skill in the art. The examples are illustrative, but do not limit the invention.

### EMBODIMENTS

Those skilled in the art will recognize, or be able to ascertain using no more than routine experimentation, many equivalents of the specific embodiments of the disclosure described herein. Such equivalents are intended to be encompassed by the following embodiments (E).

E1. A method of preparing a cell lysate, the method comprising lysing host cells and precipitating host cell DNA released from the lysed host cells to produce a lysate comprising a flocculant and a supernatant.

E2. The method of E1, wherein the host cells are lysed chemically.

E3. The method of E1 or E2, wherein the host cell membrane and nuclear membrane are lysed.

E4. The method of any one of E1-E3, wherein the host cells are suspended in a physiologically compatible fluid and are lysed chemically by adding to the fluid a solution comprising a detergent in a concentration sufficient to cause cell lysis.

E5. The method of E4, wherein the fluid and solution comprising the detergent are mixed at room temperature (18° C. to 25° C.).

E6. The method of E5, wherein mixing occurs by rocking or stirring.

E7. The method of any one of E4-E6, wherein the concentration of the detergent is sufficient to precipitate the host cell DNA.

E8. The method of any one of E4-E7, further comprising adding a solution comprising a modifier to the fluid, and optionally wherein the modifier is a salt.

E9. The method of E8, wherein the modifier is ZnSO₄, MgSO₄, MgCl₂, NaCl, sodium citrate, sodium acetate, ammonium acetate, arginine, low molecular weight dextran, sucrose octasulfate (SO) or chondroitin sulfate E (CSE).

E10. The method of E8 or E9, wherein the fluid and solution comprising the modifier are mixed at room temperature (18° C. to 25° C.) E11. The method of E10, wherein mixing occurs by rocking or stirring.

E12. The method of any one of E8-E11, wherein the solution comprising the detergent is added to the fluid before a solution comprising a modifier is added to the fluid.

E13. The method of any one of E8-E11, wherein the solution comprising the modifier is added to the fluid before the solution comprising the detergent is added to the fluid.

E14. The method of any one of E8-E13, wherein the period of time between the step of adding the solution comprising the modifier to the fluid and the step of adding the solution comprising the detergent to the fluid is 10 minutes or less.

E15. The method of any one of E4-E14, wherein the physiologically compatible fluid is growth medium.

E16. The method of any one of E4-E15, wherein the host cells are grown or maintained as an adherent cell culture on a substrate and are lysed chemically by contacting them with the solution comprising the detergent in a concentration sufficient to cause lysis, or are first detached from their substrate and suspended in the physiologically compatible fluid to which the solution comprising the detergent in a concentration sufficient to cause lysis is added.

E17. The method of any one of E1-E16, wherein the host cells are grown in suspension.

E18. The method of any one of E1-E17, wherein the host cells are mammalian cells, such as HEK293 cells, CHO cells or HeLa cells, or insect cells, such as Sf9 cells or Sf1 cells.

E19. The method of any one of E1-E18, wherein the host cells are HEK293 cells.

E20. The method of any one of E1-E19, wherein prior to lysis, the host cells have a viable cell density in the physiologically compatible fluid of at least or about 5×10⁶, 6×10⁶, 7×10⁶, 8×10⁶, 9×10⁶, 10×10⁶, 11×10⁶, 12×10⁶, 13×10⁶, 14×10⁶, 15×10⁶, 16×10⁶, 17×10⁶, 18×10⁶, 19×10⁶, 20×10⁶, 21×10⁶, 22×10⁶, 23×10⁶, 24×10⁶, 25×10⁶, 26×10⁶, 27×10⁶, 28×10⁶, 29×10⁶, or 30×10⁶ viable cells per mL (vc/mL).

E21. The method of any one of E1-E20, wherein prior to lysis, the host cells have a viable cell density in the physiologically compatible fluid of a range including and between any two of the values of E20, such as about 8×10⁶ to 15×10⁶ vc/mL, 10×10⁶ to 30×10⁶ vc/mL, 10×10⁶ vc/mL to 20×10⁶ vc/mL, 15×10⁶ to 25×10⁶ vc/mL, or 18×10⁶ to 22×10⁶ vc/mL.

E22. The method of any one of E4-E21, wherein the final concentration of detergent in the lysate is at least or about 0.005%, 0.0075%, 0.01%, 0.015%, 0.02%, 0.025%, 0.03%, 0.04%, 0.05%, 0.06%, 0.07%, 0.075%, 0.08%, 0.09%, 0.10%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1.00%, 2.00%, 3.00%, 4.00%, or 5.00% (w/v).

E23. The method of any one of E4-E22, wherein the final concentration of detergent in the lysate has a concentration range including and between any two of the values of E22, such as from about 0.005% to 5.00%, 0.005% to 1.0%, 0.01% to 1.0%, 0.01% to 0.075%, 0.02% to 0.06%, 0.025% to 0.075%, 0.04% to 0.06%, 0.045% to 0.065%, 0.05% to 5.00%, 0.10% to 2.50%, 0.20% to 1.25%, 0.20% to 0.75%, 0.25% to 0.75%, 0.25% to 0.65%, 0.20% to 0.70%, 0.30% to 0.70%, 0.35% to 0.65%, 0.40% to 0.60%, or 0.45% to 0.55% (w/v).

E24. The method of any one of E4-E23, wherein the detergent is cetyltrimethylammonium bromide (CTAB).

E25. The method of any one of E4-E24, wherein the final concentration of CTAB in the lysate ranges from 0.01% to 0.1% (w/v), from 0.025% to 0.075% (w/v), or from 0.04% to 0.06% (w/v) and optionally 0.05% (w/v) or from 0.1% to 1.0% (w/v), from 0.25% to 0.75% (w/v), or from 0.4% to 0.6% (w/v) and optionally 0.5% (w/v).

E26. The method of E24 or E25, wherein prior to lysis, the viable cell density of the host cells in the physiologically compatible fluid is at least about 10×10⁶ vc/mL and the final concentration of CTAB in the lysate is at least about 0.01% (w/v).

E27. The method of any one of E24-E26, wherein prior to lysis, the viable cell density of the host cells in the physiologically compatible fluid is at least about 10×10⁶ vc/mL, and the final concentration of CTAB in the lysate relative to the viable cell density prior to lysis is at least about 0.001% per 1×10⁶ vc/mL, and optionally about 0.005% per 1×10⁶ vc/mL.

E28. The method of any one of E24-E27, wherein the final concentration of CTAB in the lysate relative to the viable cell density prior to lysis is about 0.0005% to 0.5% per 1×10⁶ vc/mL, 0.0005% to 0.1% per 1×10⁶ vc/mL, 0.001% to 0.1%, per 1×10⁶ vc/mL, 0.001% to 0.0075% per 1×10⁶ vc/mL, 0.002% to 0.006% per 1×10⁶ vc/mL, 0.0025% to 0.0075% per 1×10⁶ vc/mL, 0.004% to 0.006% per 1×10⁶ vc/mL, 0.0045% to 0.0065% per 1×10⁶ vc/mL, 0.005% to 0.5% per 1×10⁶ vc/mL, 0.01% to 0.25% per 1×10⁶ vc/mL, 0.02% to 0.125% per 1×10⁶ vc/mL, 0.02% to 0.075% per 1×10⁶ vc/mL, 0.025% to 0.075% per 1×10⁶ vc/mL, 0.025% to 0.065% per 1×10⁶ vc/mL, 0.02% to 0.07% per 1×10⁶ vc/mL, 0.03% to 0.07% per 1×10⁶ vc/mL, 0.035% to 0.065% per 1×10⁶ vc/mL, 0.04% to 0.06% per 1×10⁶ vc/mL, or about 0.045% to 0.055 per 1×10⁶ vc/mL.

E29. The method of any one of E8-E28, wherein the final concentration of modifier in the lysate is at least or about 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 110 mM, 120 mM, 130 mM, 140 mM, 150 mM, 160 mM, 170 mM, 180 mM, 190 mM, 200 mM, 225 mM, 250 mM, 275 mM, 300 mM, 325 mM, 350 mM, 375 mM, 400 mM, 425 mM, 450 mM, 475 mM, 500 mM, 600 mM, 700 mM, 800 mM or 900 mM.

E30. The method of any one of E8-E29, wherein the final concentration of modifier in the lysate has a concentration range including and between any two of the values of E29, such as from about 1 mM to 500 mM, 10 mM to 400 mM, 10 mM to 200 mM, 10 mM to 90 mM, 25 mM to 300 mM, 25 mM to 200 mM, 25 mM to 100 mM, 25 mM to 75 mM, 40 mM to 300 mM, 40 mM to 100 mM, 40 mM to 60 mM, 45 mM to 55 mM, 50 mM to 75 mM, 50 mM to 100 mM, 50 mM to 150 mM or 50 mM to 60 mM.

E31. The method of any one of E8-E30, wherein the modifier is MgSO₄.

E32. The method E31, wherein the final concentration of MgSO₄ in the lysate ranges from 10 mM to 90 mM, from 40 mM to 60 mM, and optionally is 50 mM.

E33. The method of E31 or E32, wherein prior to lysis, the viable cell density of the host cells in the physiologically compatible fluid is at least about 10×10⁶ vc/mL and the final concentration of MgSO₄ in the lysate is at least about 50 mM.

E34. The method of any one of E31-E33, wherein prior to lysis, the viable cell density of the host cells in the physiologically compatible fluid is at least about 10×10⁶ vc/mL, and the final concentration of MgSO₄ in the lysate relative to the viable cell density prior to lysis is at least about 2.5 mM per 1×10⁶ vc/mL, and optionally about 5.0 mM per 1×10⁶ vc/mL.

E35. The method of any one of E31-E34, wherein the final concentration of MgSO₄ in the lysate relative to the viable cell density prior to lysis is about 0.1 mM to 50 mM per 1×10⁶ vc/mL, 1 mM to 40 mM per 1×10⁶ vc/mL, 1 mM to 20 mM per 1×10⁶ vc/mL, 1 mM to 9 mM per 1×10⁶ vc/mL, 2.5 mM to 20 mM per 1×10⁶ vc/mL, 2.5 mM to 20 mM per 1×10⁶ vc/mL, 2.5 mM to 10 mM per 1×10⁶ vc/mL, 2.5 mM to 7.5 mM per 1×10⁶ vc/mL, 4.0 mM to 30 mM per 1×10⁶ vc/mL, 4.0 mM to 10 mM per 1×10⁶ vc/mL, 4.0 mM to 6.0 mM per 1×10⁶ vc/mL, 4.5 mM to 5.5 mM per 1×10⁶ vc/mL, 5.0 to 7.5 mM per 1×10⁶ vc/mL, 5.0 mM to 10 mM per 1×10⁶ vc/mL, 5.0 mM to 15 mM per 1×10⁶ vc/mL or about 5.0 mM to 6.0 mM per 1×10⁶ vc/mL.

E36. The method of any one of E31-E35, wherein prior to lysis, the viable cell density of the host cells in the physiologically compatible fluid is at least about 10×10⁶ vc/mL, the final concentration of CTAB in the lysate is at least about 0.05% (w/v) and the final concentration of MgSO₄ in the lysate is at least about 50 mM.

E37. The method of any one of E31-E36, wherein prior to lysis, the viable cell density in the physiologically compatible fluid ranges from about 10×10⁶ vc/mL to 30×10⁶ vc/mL or 10×10⁶ vc/mL to 20×10⁶ vc/mL, the final concentration of CTAB in the lysate ranges from about 0.01% to 0.1%, from about 0.01% to 0.5% or from about 0.1% to 1.0%, and the final concentration of MgSO₄ in the lysate ranges from about 10 mM to 90 mM, from about 25 mM to 75 mM or about 40 mM to 60 mM.

E38. The method of any one of E31-E37, wherein prior to lysis, the viable cell density in the physiologically compatible fluid ranges from about 10×10⁶ vc/mL to 20×10⁶ vc/mL, the final concentration of CTAB in the lysate ranges from about 0.01% to 0.1%, and the final concentration of MgSO₄ in the lysate ranges from about 40 mM to 60 mM.

E39. The method of any one of E31-E38, wherein prior to lysis, the viable cell density in the physiologically compatible fluid ranges from about 10×10⁶ vc/mL to 20×10⁶ vc/mL, the final concentration of CTAB in the lysate ranges from about 0.1% to 1.0%, and the final concentration of MgSO₄ in the lysate ranges from about 40 mM to 60 mM.

E40. The method of any one of E31-E39, wherein prior to lysis, the viable cell density of the host cells in the physiologically compatible fluid is at least about 10×10⁶ vc/mL, the final concentration of CTAB in the lysate relative to the viable cell density prior to lysis is at least about 0.001% per 1×10⁶ vc/mL, and the final concentration of MgSO₄ in the lysate relative to the viable cell density prior to lysis is at least 2.5 mM per 1×10⁶ vc/mL.

E41. The method of any one of E31-E40, wherein prior to lysis, the viable cell density of the host cells in the physiologically compatible fluid is at least about 10×10⁶ vc/mL, the final concentration of CTAB in the lysate relative to the viable cell density prior to lysis is at least about 0.005% per 1×10⁶ vc/mL, and the final concentration of MgSO₄ in the lysate relative to the viable cell density prior to lysis is at least 5.0 mM per 1×10⁶ vc/mL.

E42. The method of any one of E31-E41, wherein prior to lysis, the viable cell density of the host cells in the physiologically compatible fluid is at least about 10×10⁶ vc/mL, the final concentration of CTAB in the lysate relative to the viable cell density prior to lysis is at least about 0.05% per 1×10⁶ vc/mL, and the final concentration of MgSO₄ in the lysate relative to the viable cell density prior to lysis is at least 5.0 mM per 1×10⁶ vc/mL.

E43. The method of any one of E8-E42, further comprising adding a solution comprising octoxynol-9 to the fluid followed by mixing.

E44. The method of E43, wherein the final concentration of the octoxynol-9 in the lysate ranges from 0.1% to 1.0% (w/v), and optionally 0.5% (w/v).

E45. The method of any one of E8-E44, further comprising adding a solution comprising domiphen bromide to the fluid followed by mixing.

E46. The method of E45, wherein the final concentration of the domiphen bromide in the lysate ranges from 0.07% to 0.7% (w/v), and optionally 0.2% (w/v).

E47. The method of any one of E1-E46, wherein the host cells are in a cell suspension and wherein the volume of the cell suspension at the time of lysis is at least or about 1 liter (L), 2 L, 5 L, 10 L, 20 L, 50 L, 100 L, 200 L, 250 L, 500 L, 1000 L, 1500 L, 2000 L, or more, or a range including and between any of the foregoing values, such as 1 L to 100 L, 50 L to 500 L, 500 L to 1000 L, 500 L to 1500 L, 1000 L to 1500 L, or 500 L to 2000 L which, in some embodiments, is enclosed within a container, such as a tank, bag, or bioreactor.

E48. The method of any one of E8-E47, wherein the fluid comprising i) the solution comprising a modifier, ii) the solution comprising the detergent or iii) both is mixed for at least or about 5 minutes (mins), 10 mins, 15 mins, 20 mins, 30 mins, 40 mins, 50 mins, 60 mins, 70 mins, 75 mins, 80 mins, 90 mins, 100 mins, 115 mins, 120 mins, 150 mins, 180 mins, or a range of time including and between any two of the foregoing values

E49. The method of any one of E48, wherein mixing occurs by rocking or stirring.

E50. The method of any one of E1-E49, further comprising separation of the supernatant from the flocculant by filtration to produce a clarified lysate.

E51. The method of E50, wherein during filtration the supernatant and flocculant are mixed by stirring.

E52. The method of E50 or E51, wherein the filtration is performed using a membrane filter having an average pore size of less than or equal to about 100 μm, 50 μm, 40 μm, 30 μm, 25 μm, 20 μm, 15 μm 10 μm, 5 μm, 2 μm, 1 μm, 0.5 μm, 0.2 μm, or 0.1 μm.

E53. The method of any one of E50-E52, wherein the filtration is depth filtration, ultrafiltration, diafiltration of a combination thereof.

E54. The method of any one of E1-E53, wherein the host cells produce a biological product.

E55. The method of E54, further comprising purifying the biological product from the supernatant by at least one downstream processing step.

E56. The method of E55, wherein the downstream processing step comprises chromatography.

E57. The method of E56, wherein the downstream processing step is selected from the group consisting of affinity chromatography (AF), anion-exchange chromatography (AEX), cation exchange chromatography (CEX), hydrophobic interaction chromatography (HIC), immunoaffinity chromatography, pseudoaffinity chromatography, size exclusion chromatography (SEC) and a combination thereof.

E58. The method of any one of E54-E57, wherein the biological product produced by the host cells is recombinant.

E59. The method of any one of E54-E58, wherein the biological product produced by the host cells is a virus particle.

E60. The method of E59, wherein the virus particle is an adenovirus particle, an adeno-associated virus (AAV) particle, a retrovirus particle or a lentivirus particle.

E61. The method of any one of E59 or E60, wherein the virus particle is modified to express a heterologous gene from a viral vector.

E62. The method of E61, wherein the viral vector is an AAV vector or a recombinant AAV (rAAV) vector.

E63. The method of E62, wherein the AAV vector or rAAV vector comprise a capsid serotype selected from the group consisting of AAV1, AAV2, AAV3 (including AAV3A and AAV3B), AAV4, AAV5, AAV6, AAV7, AAV8, AAV9, AAV10, AAV12, AAV13, AAVrh8, AAVRh10, AAV rh32.22, AAVRh39, AAVRh43, Rh74, AAV-DJ, AAV-PHP.B, Anc80, AAV1.1, AAV2.5, AAV6.1, AAV6.2, AAV6.3.1, AAV9.45, AAV2i8, AAVShH10, HSC15/17, RHM4-1, RHM15-1, RHM15-2, RHM15-3/RHM15-5, RHM15-4, RHM15-6, AAVhu.26, AAV29G, AAV-LK03, AAV2-TT, AAV2-TT-S312N, AAV3B-S312N, avian AAV, bovine AAV, canine AAV, equine AAV, primate AAV, non-primate AAV, and ovine AAV, NP4, NP22, NP66, AAVDJ/8, and AAVDJ/9.

E64. The method of E62 or E63, wherein the AAV vector or rAAV vector comprise a capsid that binds to heparan sulfate proteoglycan (HSPG) receptors.

E65. The method of E65, wherein the AAV vector or rAAV vector comprise an AAV2, AAV3 (including AAV3A and AAV3B), AAV6, AAV13 or AAV-DJ capsid.

E66. The method of E63, wherein the AAV vector or the rAAV vector comprise an AAV9 capsid.

E67. The method of any one of E62-E66 wherein the rAAV vector comprises a vector genome (vg).

E68. The method of E67, wherein the vector genome comprises a heterologous transgene, optionally for the expression of a therapeutic polypeptide.

E69. The method E68, wherein the heterologous transgene encodes a therapeutic polypeptide for the treatment of Duchenne muscular dystrophy, Gaucher disease, Wilson disease, Hemophilia A or Hemophilia B.

E70. The method of E69, wherein the heterologous transgene encodes a therapeutic polypeptide comprising the amino acid sequence of any one of SEQ ID NO:1, SEQ ID NO:2 or SEQ ID NO:3.

E71. The method of any one of E67-E70, wherein the amount of rAAV vector in the clarified harvest, in an affinity chromatography pool, in an AEX chromatography pool, in a drug substance, in a drug product or a combination thereof is measured by quantitative PCR (QPCR) analysis of the heterologous transgene within the vector genome (vg).

E72. The method E71, wherein the clarified lysate comprises about 1.5 fold to 10 fold more, 1.5 fold to 7 fold more or 1.5 fold to 5 fold more vg as compared to a clarified lysate prepared using octoxynol-9 and domiphen bromide to lyse the host cells and precipitate the host cell DNA.

E73. The method of E71, wherein the clarified lysate comprises about or at least 1×10¹³ vg/liter host cell culture (vg/L), 1.5×10¹³ vg/L, 2×10¹³ vg/L, 2.5×10¹³ vg/L, 3.0×10¹³ vg/L, 3.5×10¹³ vg/L, 4.0×10¹³ vg/L, 4.5×10¹³ vg/L, 5.0×10¹³ vg/L, 5.5×10¹³ vg/L, 6.0×10¹³ vg/L, 6.5×10¹³ vg/L, 7.0×10¹³ vg/L, 7.5×10¹³ vg/L, 8.0×10¹³ vg/L, 8.5×10¹³ vg/L, 9.0×10¹³ vg/L, 9.5×10¹³ vg/L, 1×10¹⁴ vg/L, 2×10¹⁴ vg/L, 3×10¹⁴ vg/L, 4×10¹⁴ vg/L or 5×10¹⁴ vg/L or more, or a range including and between any two of the foregoing values, such as about 1×10¹³ to 5×10¹³ vg/L, 4×10¹³ to 9×10¹³ vg/L, 8×10¹³ to 3×10¹⁴ vg/L or 2×10¹⁴ to 5×10¹⁴ vg/L.

E74. The method of any one of E71-E73, wherein the clarified lysate comprises about or at least 1.0×10² ng host cell DNA (HCD)/1×10⁹ vector genomes (vgs), 2.0×10² ng HCD/1×10⁹ vgs, 3.0×10² ng HCD/1×10⁹ vgs, 4.0×10² ng HCD/1×10⁹ vgs, 5.0×10² ng HCD/1×10⁹ vgs, 6.0×10² ng HCD/1×10⁹ vgs, 7.0×10² ng HCD/1×10⁹ vgs, 8.0×10² ng HCD/1×10⁹ vgs, 9.0×10² ng HCD/1×10⁹ vgs, 1.0×10³ ng HCD/1×10⁹ vgs, 2.0×10³ ng HCD/1×10⁹ vgs, 3.0×10³ ng HCD/1×10⁹ vgs, 4.0×10³ ng HCD/1×10⁹ vgs, 5.0×10³ ng HCD/1×10⁹ vgs, 1.0×10⁴ ng HCD/1×10⁹ vgs, 5×10⁴ ng HCD/1×10⁹ or more, or a range including and between any two of the foregoing values, such as about 1.0×10² ng HCD/1×10⁹ vgs to 5.0×10² ng HCD/1×10⁹ vgs, 4.0×10² ng HCD/1×10⁹ vgs to 9.0×10² ng HCD/1×10⁹ vgs, 8.0×10² ng HCD/1×10⁹ vgs to 3.0×10³ ng HCD/1×10⁹ vgs, 2.0×10³ ng HCD/1×10⁹ vgs to 7.0×10³ ng HCD/1×10⁹ vgs, or 6.0×10³ ng HCD/1×10⁹ vgs to 1.0×10⁴ ng HCD, or 8.0×10³ ng HCD/1×10⁹ to 5.0×10⁴ ng HCD/1×10⁹.

E75. The method of any one of E50-E74, wherein the clarified lysate comprises about or at least 1.0×10⁶ pg host cell DNA (HCD)/mL lysate, 2.0×10⁶ pg HCD/mL lysate, 3.0×10⁶ pg HCD/mL lysate, 4.0×10⁶ pg HCD/mL lysate, 5.0×10⁶ pg HCD/mL lysate, 6.0×10⁶ pg HCD/mL lysate, 7.0×10⁶ pg HCD/mL lysate, 8.0×10⁶ pg HCD/mL lysate, 9.0×10⁶ pg HCD/mL lysate, 1.0×10⁷ pg HCD/mL lysate, 2.0×10⁷ pg HCD/mL lysate, 3.0×10⁷ pg HCD/mL lysate, 4.0×10⁷ pg HCD/mL lysate, 5.0×10⁷ pg HCD/mL lysate, 1.0×10⁸ pg HCD/mL lysate, 5×10⁸ pg HCD/mL lysate or more, or a range including and between any two of the foregoing values, such as about 1.0×10⁶ pg HCD/mL lysate to 5.0×10⁶ pg HCD/mL lysate, 4.0×10⁶ pg HCD/mL lysate to 9.0×10⁶ pg HCD/mL lysate, 8.0×10⁶ pg HCD/mL lysate to 3.0×10⁷ pg HCD/mL lysate or 2.0×10⁷ pg HCD/mL lysate to 5.0×10⁷ pg HCD/mL lysate.

E76. The method of any one of E67-E75, wherein the rAAV vector is further purified from the clarified lysate by affinity chromatography (AF) to produce an AF pool and wherein the affinity chromatography pool comprises 1.5 to 5 fold more, 1.5 fold to 7 fold more or 1.5 fold to 10 fold more vector genomes as compared to an AF pool prepared using octoxynol-9 and domiphen bromide to lyse the host cells and precipitate the host cell DNA.

E77. The method of any one of E67-E75, wherein the rAAV vector is further purified from the clarified lysate by affinity chromatography (AF) to produce an AF pool and wherein the AF pool comprises 50 ug/mL or less of residual CTAB, 1% or less high molecular mass species (HMMS), 30% or more full capsids, or a combination thereof.

E78. The method of any one of E67-E75, wherein the rAAV vector is further purified from the clarified lysate by affinity chromatography (AF) to produce an AF pool and wherein the AF pool comprises about or at least 1×10¹³ vg/liter host cell culture (vg/L), 1.5×10¹³ vg/L, 2×10¹³ vg/L, 2.5×10¹³ vg/L, 3.0×10¹³ vg/L, 3.5×10¹³ vg/L, 4.0×10¹³ vg/L, 4.5×10¹³ vg/L, 5.0×10¹³ vg/L, 5.5×10¹³ vg/L, 6.0×10¹³ vg/L, 6.5×10¹³ vg/L, 7.0×10¹³ vg/L, 7.5×10¹³ vg/L, 8.0×10¹³ vg/L, 8.5×10¹³ vg/L, 9.0×10¹³ vg/L, 9.5×10¹³ vg/L, 1×10¹⁴ vg/L, 2×10¹⁴ vg/L, 3×10¹⁴ vg/L, 4×10¹⁴ vg/L or 5×10¹⁴ vg/L or more, or a range including and between any two of the foregoing values, such as about 1×10¹³ to 5×10¹³ vg/L, 4×10¹³ to 9×10¹³ vg/L, 8×10¹³ to 3×10¹⁴ vg/L or 2×10¹⁴ to 5×10¹⁴ vg/L.

E79. The method of any one of E67-E75, wherein the rAAV vector is further purified from the clarified lysate by affinity chromatography (AF) to produce an AF pool and wherein 96%, 97%, 98%, 99% or 100% or a range including and between 96% to 100% of the rAAV vector in the AF pool in a monomer.

E80. The method of any one of E67-E75, wherein the rAAV vector is further purified from the clarified lysate by affinity chromatography (AF) to produce an AF pool and wherein the AF pool comprises about or less than 5.0 pg host cell DNA (HCD)/1×10⁹ vector genome (vg), 10 pg HCD/1×10⁹ vg, 15 pg HCD/1×10⁹ vg, 20 pg HCD/1×10⁹ vg, 25 pg HCD/1×10⁹ vg, 30 pg HCD/1×10⁹ vg, 35 pg HCD/1×10⁹ vg, 40 pg HCD/1×10⁹ vg, 45 pg HCD/1×10⁹ vg, 50 pg HCD/1×10⁹ vg, 100 pg HCD/1×10⁹ vg, 200 pg HCD/1×10⁹ vg, 300 pg HCD/1×10⁹ vg or 500 pg HCD/1×10⁹ vg.

E81. The method of any one of E76-E80, wherein the rAAV vector is further purified from the AF pool by AEX to produce an AEX pool, and wherein the AEX pool comprises 50 ug/mL or less of residual CTAB, 2% or less high molecular mass species (HMMS), 50% or more full capsids or a combination thereof.

E82. The method of any one of E76-E80, wherein the rAAV vector is further purified from the AF pool by AEX to produce an AEX pool, and wherein the AEX pool comprises about or at least 1×10¹³ vg/liter host cell culture (vg/L), 1.5×10¹³ vg/L, 2×10¹³ vg/L, 2.5×10¹³ vg/L, 3.0×10¹³ vg/L, 3.5×10¹³ vg/L, 4.0×10¹³ vg/L, 4.5×10¹³ vg/L, 5.0×10¹³ vg/L, 5.5×10¹³ vg/L, 6.0×10¹³ vg/L, 6.5×10¹³ vg/L, 7.0×10¹³ vg/L, 7.5×10¹³ vg/L, 8.0×10¹³ vg/L, 8.5×10¹³ vg/L, 9.0×10¹³ vg/L, 9.5×10¹³ vg/L, 1×10¹⁴ vg/L, 2×10¹⁴ vg/L, 3×10¹⁴ vg/L, 4×10¹⁴ vg/L or 5×10¹⁴ vg/L or more, or a range including and between any two of the foregoing values, such as about 1×10¹³ to 5×10¹³ vg/L, 4×10¹³ to 9×10¹³ vg/L, 8×10¹³ to 3×10¹⁴ vg/L or 2×10¹⁴ to 5×10¹⁴ vg/L.

E83. The method of any one of E76-E80, wherein the rAAV vector is further purified from the AF pool by AEX to produce an AEX pool and wherein 96%, 97%, 98%, 99% or 100%, or a range including and between 96% to 100% of the rAAV vector in the AEX pool in a monomer.

E84. The method of any one of E81-E83 wherein the rAAV vector is further purified from the AEX pool by ultrafiltration, diafiltration or both to produce a drug substance, and wherein the drug substance comprises 50 ug/mL or less of residual CTAB, 2% or less high molecular mass species (HMMS), 60% or greater full capsids or a combination thereof.

E85. The method of any one of E81-E83, wherein the rAAV vector is further purified from the AEX pool by ultrafiltration, diafiltration or both to produce a drug substance and wherein the drug substance comprises about or at least 1×10¹³ vg/liter host cell culture (vg/L), 1.5×10¹³ vg/L, 2×10¹³ vg/L, 2.5×10¹³ vg/L, 3.0×10¹³ vg/L, 3.5×10¹³ vg/L, 4.0×10¹³ vg/L, 4.5×10¹³ vg/L, 5.0×10¹³ vg/L, 5.5×10¹³ vg/L, 6.0×10¹³ vg/L, 6.5×10¹³ vg/L, 7.0×10¹³ vg/L, 7.5×10¹³ vg/L, 8.0×10¹³ vg/L, 8.5×10¹³ vg/L, 9.0×10¹³ vg/L, 9.5×10¹³ vg/L, 1×10¹⁴ vg/L, 2×10¹⁴ vg/L, 3×10¹⁴ vg/L, 4×10¹⁴ vg/L or 5×10¹⁴ vg/L or more, or a range including and between any two of the foregoing values, such as about 1×10¹³ to 5×10¹³ vg/L, 4×10¹³ to 9×10¹³ vg/L, 8×10¹³ to 3×10¹⁴ vg/L or 2×10¹⁴ to 5×10¹⁴ vg/L.

E86. A biological product produced by the method of any one of E1-E85.

E87. The biological product of E86, wherein the biological product is a viral vector. E88. The biological product of E86, wherein the biological product is an AAV vector or a rAAV vector.

E89. A composition comprising a biological product produced by the method of E1-E85

E90. The composition of E89, wherein the biological product is a viral vector.

E91. The composition of E89, wherein the biological product is an AAV vector or a rAAV vector.

E92. The composition of E91, wherein the capsids of the AAV vector or rAAV vector in the composition are at least 20%, 30%, 40%, 50%, 60%, 70% or 80% full.

E93. The composition of any one of E89-E92, wherein the composition is a pharmaceutical composition.

E94. The composition of E93, wherein the pharmaceutical composition comprises an excipient or additive.

E95. A composition comprising the drug substance of any one of E84 or E85.

E96. A system for performing the method of E4 or E5, the system comprising: (i) a means for containing the host cells suspended in the physiologically compatible fluid, ii) a means for containing the detergent, and (iii) a means for mixing the fluid and detergent.

E97. A system for performing the method of E8, the system comprising: (i) a means for containing the host cells suspended in the physiologically compatible fluid, ii) a means for containing the detergent, iii) a means for containing the modifier and (iv) a means for mixing the fluid, the detergent and the modifier.

E98. A method of preparing a cell lysate, the method comprising lysing host cells and precipitating DNA released from the host cells to produce a lysate comprising a flocculant and a supernatant,
wherein the host cells are suspended in a physiologically compatible fluid and are lysed chemically by adding to the fluid a solution comprising a detergent in a concentration sufficient to cause cell lysis and flocculation of host cell DNA.

E99. The method of E98, further comprising adding to the fluid a solution comprising a modifier.

E100. The method of E99, wherein the solution comprising the modifier is added to the fluid before the solution comprising the detergent is added to the fluid.

E101. The method of E100, wherein the solution comprising the modifier is added to the fluid followed by a mixing for at least 5 minutes to produce a mixture.

E102. The method of E101, wherein the solution comprising the detergent is added to the mixture followed by mixing for at least 5 minutes to produce the flocculant and the supernatant.

E103. The method of any one of E99-E102, wherein the physiologically compatible fluid, the solution comprising the detergent and the solution comprising the modifier are mixed by rocking or stirring for 5 minutes to 60 minutes, optionally for 30 minutes at room temperature of 18° C. to 25° C.

E104. The method of any one of E98-E103, further comprising filtration of the supernatant and flocculant to produce a clarified lysate.

E105. A method of preparing a cell lysate, the method comprising lysing host cells and precipitating DNA released from the host cells to produce a lysate comprising a flocculant and a supernatant,
wherein the host cells are suspended in a physiologically compatible fluid and are lysed chemically by adding to the fluid a solution comprising a modifier to form a mixture and adding to the mixture a solution comprising a detergent, and
wherein the modifier and the detergent are in concentrations sufficient to cause cell lysis and flocculation of host cell DNA.

E10⁶. The method of E105, wherein the modifier is MgSO₄ and the detergent is CTAB. E107. The method of E10⁶, wherein the concentration of MgSO₄ is about 25 mM to 75 mM, or optionally about 50 mM and wherein the concentration of CTAB is about 0.01% to 0.1% (w/v), and optionally 0.05% (w/v).

E108. The method of any one of E105 to E107, wherein the flocculant and supernatant are separated by filtration to produce a clarified lysate.

E109. The method of any one of E1-E85 and E98-E108, further comprising adding to the fluid a solution comprising octoxynol-9 in a concentration sufficient to cause cell lysis. E110. The method of any one of E1-E85 and E98-E108, further comprising adding to the fluid a solution comprising domiphen bromide in a concentration sufficient to cause flocculation of host cell DNA.

### EXAMPLES

The productivity of an rAAV purification process (Process 1) was evaluated to improve the total productivity (vg/L) as well as the productivity at particular points in the process (e.g., at affinity chromatography elution pool, at anion exchange chromatography elution pool). The affinity chromatography elution pool step was the earliest point in the process where productivity could be assessed. Data from analysis of the clarified lysate (post depth and absolute filter material) was also generated. The Examples below describe development and testing of an improved harvest method (Process 2) that resulted in increased productivity as measured as vector genomes (vg)/L bioreactor and increased percentage of full capsids at the affinity pool and AEX pool steps.

TABLE 1
Process 1 average attribute values and Process 2 target attribute values

| | | Process 1 Average | Process 2 Target |
|---|---|---|---|
| Step | Parameter / Attribute | values | values |
| Harvest | Chemical composition | 0.5% w/v octoxynol-9 / | Area for Development |
| method | | w/v 0.2% domiphen | |
| | | bromide (DB) | |
| Affinity | Productivity (vg/L | 1.6E+13 | >7.0E+13 |
| Pool | bioreactor) | | |
| | % Full capsid | 14% | >22% |
| | Host cell DNA (HCDNA) | 123 ± 70 | In line with Process 1 |
| | (pg/1E+9 vg) | | |
| | Host cell protein (HCP) | 88 | In line with Process 1 |
| | (pg/1E+9 vg) | | |
| AEX | Productivity (vg/L | 1.1E+13 | >5.5E+13 |
| Pool | bioreactor) | | |
| | % Full capsid | 25% | >52% |

### Example 1: Small Scale Screening Evaluation of Cetyltrimethylammonium Bromide (CTAB) as an Alternative to Octoxynol-9 Harvest Method

Based on prior observations using a high salt flush of post lysis cell debris, it was determined that a substantial amount of rAAV3B vector remained bound to cell debris during lysis and flocculation with octoxynol-9 and domiphen bromide (DB) and was subsequently lost during filtration to produce the clarified lysate. This Example demonstrates that a lysis buffer comprising CTAB reduced the amount of rAAV vector that was bound to cell debris and thereby resulted in higher vector titers at both clarified lysate and loose resin affinity elution pool steps.

HEK293 cells were grown in suspension culture and transfected with 2 plasmids to produce a rAAV3B vector using standard methods known in the art. HEK293 cells were harvested, lysed, flocculated, and the resulting lysate was filtered to produce a clarified lysate (also referred to as a clarified cell lysate or a clarified harvest). The rAAV vector contained a vector genome with a transgene encoding a polypeptide comprising the amino acid sequence of SEQ ID NO:1 (copper transporting ATPase 2 with deletion of metal binding sites 1-4).

Initially, a screen was performed to evaluate alternative detergents for cell lysis and to drive higher rAAV vector recovery in the presence or absence of additional modifiers, such as 0.7 M sodium chloride and 0.2M arginine. The combination of detergents, with or without additional modifiers, were tested using low (approximately 12E+6 cells/mL culture) and high (approximately 18E+6 cells/mL culture) cell density harvests (LCD and HCD respectively). Detergents tested included: sodium dodecyl sulfate (SDS), polysorbate 20 (PS20), sarkosyl, deoxycholic acid sodium salt (DOC), CTAB, nonylphenyl ether (NP-40), and 3-[(3-cholamidopropyl)dimethylammonio]-1-propanesulfonate (CHAPS). All detergents except for CTAB were tested in the presence of 0.7M sodium chloride and separately in the presence of 0.2M Arginine. In the follow up screen, CTAB was tested in the presence of the following modifiers including low molecular weight and high molecular weight dextrans, sucrose octasulfate (SOS), chondroitin sulfate E (CSE), and magnesium sulfate (MgSO₄) to see if additional improvement in capsid recovery could be achieved.

All conditions were tested using 10 mL of cell culture and processed at room temperature (18° C.-25° C.) in a 15 mL tube by addition of the modifier first if present, followed by addition of the detergent being testing. If the tested condition included a modifier the cell culture was mixed for 10 minutes with rocking prior to addition of the detergent to a final concentration of 0.5% w/v. The cell culture comprising the modifier and detergent was mixed by rocking for an additional 30 minutes to lyse the cells.

Following lysis of the cells, all reactions were flocculated using 0.2% of domiphen bromide (DB) for 15 minutes while mixing on the rocker. All recoveries were calculated as total vector genomes (vg) generated relative to vg titer of a clarified lysate prepared using 0.5% octoxynol-9 and 0.2% DB (control condition). Table 2 shows the fold improvement of capsid recovery from clarified lysate across specified detergents in the presence or absence of modifiers.

TABLE 2
Fold improvement of capsid recovery in clarified lysate.

| | LCD | | | | | | LCD and HCD |
|---|---|---|---|---|---|---|---|
| Detergent | NP-40 | SDS | PS20 | CHAPS | sarkosyl | DOC | CTAB |
| No modifier | Fold improvement over octoxynol-9 and DB control | | | | | | |
| | 0.9 | 0.1 | 0.5 | 2.1 | 0.3 | 0.1 | 1.6 (LCD) 2.8 (HCD) |
| | Fold improvement over octoxynol-9 and DB control | | | | | | Extended set |
| 0.7M NaCl | 2.8 | 0.2 | 0.7 | 1.6 | n/a | n/a | of modifiers were tested in |
| | Fold improvement over octoxynol-9 and DB control | | | | | | a follow up screen (see |
| 0.2M Arginine | 1.0 | 0.9 | 1.8 | 1.0 | n/a | n/a | Table 3) |

Fold improvement is calculated by dividing total vgs (based on qPCR values) in a clarified lysate produced using a test condition over total vgs in a clarified lysate produced using in 0.5% octoxynol-9 and 0.2% DB control.

Based on the initial screening results, two detergents, CHAPS and CTAB, were selected as potential candidates for use in the production of the clarified cell lysate. Despite the high level of capsid recovery (2.8 fold improvement over control) in the study run using 0.7M sodium chloride as the modifier and 0.5% w/v NP-40 as the lysis detergent, this combination was not considered a potential process candidate due to the observed disadvantageous effects on flocculation, filtration and downstream chromatography processes. The study run using 0.2M arginine as the modifier and polysorbate 20 as the detergent also had an acceptable level of capsid recovery (1.8 fold improvement over control). Based on the positive charge of arginine, this reagent was considered a good modifier candidate. However, the combination of arginine and polysorbate 20 was not pursued further due to potential capsid aggregation issues.

CHAPS was identified as a potential detergent candidate in the small scale screens and was evaluated at a larger scale (1 L bioreactor) at a final concentration of 0.5% w/v. However, the detergent failed to provide the same recovery improvement at large scale as at small scale.

The improved vg recovery observed with CTAB detergent as compared to recovery using octoxynol-9 was demonstrated at small scale (15 mL culture) and showed improvement even at different cell densities. The recovery from a low cell density culture was 1.6 fold higher than an octoxynol-9 and DB control combination. The recovery from a high cell density culture was 2.8 fold higher than an octoxynol-9 and DB control combination. Based on these small scale screens, CTAB was selected as the detergent candidate for further development, including a follow up small scale screen of modifiers to complement CTAB to achieve even higher capsid recovery.

### Example 2: Role of Salts in CTAB Performance

To further demonstrate the potential for improved vg recovery using CTAB during the lysis step, small scale screens were performed in the presence of different modifiers. The modifiers were selected based on their potential to interfere with and inhibit non-specific binding of rAAV3B capsids to heparin sulfate proteoglycan receptors on cells and cell lysis debris. Using 10 mL cell culture aliquots, modifiers were delivered to the culture at the concentrations shown in Table 3 and mixed for 10 min at room temperature (RT) prior to addition of the CTAB lysis detergent. CTAB was added to a final concentration of 0.5% w/v. Fold improvement in productivity was calculated by dividing total vector genomes (vgs) measured by qPCR quantification of copies of the vector transgene in product produced by a test condition over total vgs produced using 0.5% octoxynol-9 and 0.2% DB as the control lysis and flocculation condition. Total vgs were measured in the clarified lysate and in eluate produced by loose resin affinity purification of the clarified lysate. Conditions which produced greater than 2-fold improvement are highlighted in bold in Table 3.

TABLE 3
Product recovery improvement in clarified
lysate and loose affinity resin elution

| | | | fold | fold |
|---|---|---|---|---|
| | | | productivity | productivity |
| | | | over | over |
| | | | octoxynol- | octoxynol- |
| | | | 9/DB | 9/DB |
| | | | control - | control - |
| Cell | Detergent | | clarified | affinity |
| Density | (0.5% w/v) | Modifier | lysate | elution |
| LCD | CTAB | — | 2.81 | 3.81 |
| | CTAB | 50 mM MgSO₄ | 3.21 | 7.77 |
| | CTAB | 6 mM ZnSO₄ | 1.86 | 2.41 |
| | CTAB | 400 ug/mL low | 0.14 | 1.20 |
| | | molecular weight | | |
| | | (LMW) Dextran | | |
| | CTAB | 80 μM SOS | 2.61 | 3.20 |
| | CTAB | 80 μM CSE | 1.22 | 0.05 |

SOS = sucrose octasulfate; CSE = chondroitin sulfate E.

The only identified condition that demonstrated an additional increase in the amount of recovered vector genome in both clarified lysate and affinity elution pool over CTAB alone (no modifier), was the combination of 0.5% w/v CTAB with 50 mM MgSO₄ (3.21 for the clarified lysate and 7.77 for the affinity pool). No other modifiers studied provided any additional increase in recovery. MgSO₄ has been demonstrated to improve harvest vg recovery, most likely by masking the charge of the highly sulfated heparan proteoglycan receptor found on cells with a Mg²⁺ ion. The impact of MgSO₄ as a modifier to a octoxynol-9/DB lysis and flocculation process was also tested to decouple the effect of CTAB from MgSO₄. The study was performed according to Wright et al. (Molecular Therapy (2005) 12(1):171). A ˜2-fold increase in productivity with both low (37 mM) and high (120 mM) concentrations of MgSO₄ was observed. However, this combination of reagents was shown not to be scalable, as addition of MgSO⁴ to the octoxynol-9/DB lysis and flocculation process posed major complications by inhibiting DB driven flocculation and by severely compromising both filtration and affinity chromatography through increased system pressure.

Based in part on these findings, process development was focused on the combination of MgSO₄ and CTAB. Also, reports in the literature indicated that the critical micelle concentration of CTAB is altered in the presence of salts such that MgSO₄ can drive the concentration of CTAB in water from about 1.04 mM to about 0.63 mM. (Adhikari et al., BIBECHANA, 2017, 14:77-85) Thus, the inventors hypothesized that an increased concentration of CTAB micelles would drive more efficient recovery of rAAV3B capsids.

### Example 3: Considerations of Potential Process Impacts from Using CTAB

Since CTAB has not been used previously in processes for the production of gene therapy vectors for clinical use, potential negative effects of the reagent, such as the ability to clear CTAB through the downstream purification processes, as well as its effect on capsid stability and infectivity, were studied (Table 4). A reverse phase-high performance liquid chromatography (RP-HPLC) method was developed to track residual CTAB through the purification process and address any clearance concerns.

Capsid stability was evaluated by testing for % HMMS levels in the affinity elution pool using a size exclusion-high performance liquid chromatography (SE-HPLC) method and found to be not more than 1% of total capsids. The quality attributes (including potency) of drug substance and drug product generated using CTAB as the lysis reagent were evaluated using 250 L scale cell cultures and were found to be comparable to drug product and drug substance obtained using octoxynol-9 as the lysis reagent and DB as the flocculant. In short, CTAB clearance down to non-detectable levels was demonstrated along with comparable % HMMS levels (as an indicator of capsid stability) and % AUC (as an indicator of % full capsids) as compared to Process 1 using octoxynol-9 and DB.

TABLE 4
Attributes of affinity pool, AEX pool and drug substance
from purification of a 250 L cell culture.

| | Residual CTAB, | | % full |
|---|---|---|---|
| Process step | [ug/mL] | % HMMS | capsids (AUC) |
| Affinity pool | 28.6 | NMT 1.0 | 48.2 |
| AEX pool | NMT 3.3 | NMT 1.0 | 78.4 |
| Drug Substance | NMT 3.3 | NMT 1.0 | 80.4 |

NMT = not more than

### Example 4: 1 L, 10 L and 250 L Bioreactor Scale Studies

Due to limited data on host cell DNA clearance and viral inactivation for CTAB alone (or in combination with MgSO₄), further studies were also conducted using CTAB i) with DB to ensure additional impurity removal and ii) with octoxynol-9 to ensure viral inactivation. The resulting chemical composition for the recovery condition was 50 mM MgSO₄, 0.25% w/v CTAB, 0.5% w/v octoxynol-9 and 0.2% w/v DB.

HEK293 cells were grown in 1 L, 10 L and 250 L suspension cultures and transfected with 2 plasmids to produce a rAAV3B vector using standard methods known in the art. The rAAV vector contained a vector genome with a transgene encoding a polypeptide comprising the amino acid of SEQ ID NO:1 (copper transporting ATPase 2 with deletion of metal binding sites 1-4). HEK293 cells were harvested, lysed and flocculated, and the resulting lysate was filtered to produce a clarified lysate. Table 5 shows the details of harvest method and observed productivities in vg/L. Cell cultures treated with 50 mM MgSO₄ and 0.5% CTAB generally demonstrated higher productivity.

TABLE 5
Side by side comparison of bioreactor productivities observed at 1 L, 10 L and 250 L cell culture scale.

| Bioreactor ID | | 1 | | 2 | | 3 | |
|---|---|---|---|---|---|---|---|
| Scale | | 1 L | | 10 L | | 250 L | |
| Clarified | Condition | 0.5% | 50 mM | 0.5% | 50 mM | 0.5% | * CTAB |
| lysate | | oxtoxynol-9, | MgSO₄, | octoxynol-9, | MgSO₄, | octoxynol-9, | combination |
| | | 0.2% DB | 0.5% CTAB | 0.2% DB | 0.5% CTAB | 0.2% DB | |
| | Productivity | 1.76E+13 | 4.29E+13 | 1.15E+13 | 2.36E+13 | 1.5e+13 | 9.39e+13 |
| | (vg/L) | | | | | | |
| Affinity | Productivity | 1.05e+13 | 4.68E+13 | 2.7E+12 | 1.95e+13 | 1.34e+13 | 5.8E+13 |
| elution | (vg/L) | | | | | | |
| Pool | % Monomer | 99.1 | 96.6 | 99.3 | 99.3 | 99.1 | 98 |
| | Affinity resin | cAVB | | | | AAVx | |
| | used | | | | | | |

* CTAB combination: CTAB/octoxynol-9 dual detergent composition comprised 50 mM MgSO₄, 0.25% CTAB, and 0.5% octoxynol-9 with 0.2% DB to ensure complete flocculation.

Chemicals were added in the order shown above. A 20 minute incubation time was allocated to 50 mM MgSO₄, with a 30-minute incubation time for each detergent and a 15-minute incubation time for DB, all with mixing.

1 L and 250 L low cell density cultures treated with MgSO₄ and CTAB (including in combination with octoxynol-9 and DB) demonstrated a ˜4-fold increase in productivity at the affinity chromatography elution pool step as compared to cultures treated with 0.5% octoxynol-9 and 0.2% DB. Product quality measured as % monomer at the affinity elution pool step was comparable for all conditions. A measure of residual CTAB in the affinity elution pool was not available for this dataset.

The 10 L culture used for evaluation was not fully representative of the upstream process as deviations were recorded. Even so, a 2-fold improvement was measurable at the clarified lysate stage. Comparison of productivity levels at the affinity elution pool step of the 10 L scale was compromised by poor performance of the 0.5% octoxynol-9/0.2% DB condition during affinity chromatography (2.7E+12). Only a 23% step yield was observed which was not in line with process history data.

A 4-fold increase in productivity was observed for cell cultures treated with 50 mM MgSO₄ and 0.5% CTAB so that condition was selected for further process development and testing.

### Example 5: 10 L, 250 L and 2000 L Bioreactor Scale Demonstration Using a High Cell Density Upstream Culture

HEK293 cells were grown in 10 L high cell density (HCD) suspension cultures and transfected with 3 plasmids to produce a rAAV3B vector using standard methods known in the art. The rAAV vector contained a vector genome with a transgene encoding a polypeptide comprising the amino acid sequence of SEQ ID NO:1 (copper transporting ATPase 2 with deletion of metal binding sites 1-4). HEK293 cells were harvested, lysed, flocculated, and the resulting lysate was filtered to produce a clarified lysate. Table 6 shows the details of harvest conditions and observed bioreactor productivities in comparison to the control condition comprising 0.5% octoxynol-9 plus 0.2% DB condition without CTAB.

TABLE 6
Summary of process data for 10 L high cell density cultures.

| | Bioreactor ID | | |
|---|---|---|---|
| | 007 | | 008 |
| | Scale | | |
| | 10 L | | 10 L |
| Harvest method | 0.5% octoxynol- | | 0.5% octoxynol- |
| | 9/0.2% DB | | 9/0.2% DB |
| | 50 mM MgSO₄/0.25% | | 50 mM MgSO₄/0.25% |
| | CTAB/0.5% octoxynol- | | CTAB/0.5% octoxynol- |
| | 9/0.2% DB | | 9/0.2% DB |
| productivity at | 4.7E+13 | vg/L | 4.99E+13 vg/L |
| clarified lysate | 9.96E+13 | vg/L | 1.03E+14 vg/L |
| productivity at | 2.26E+13 | vg/L | 2.91E+13 vg/L |
| affinity eluate pool | 1.02E+14 | vg/L | 9.73E+13 vg/L |
| % HMMS affinity | NMT 1.0 | | NMT 1.0 |
| eluate pool | 7.7% | | NMT 1.0 |
| productivity at AEX | 1.6E+13 | vg/L | 4.04E+13 vg/L |
| eluate pool | 4.95E+13 | vg/L | 8.49E+13 vg/L |

| | | |
|---|---|---|
| % HMMS AEX | 1.4% | 1.2% |
| eluate pool | NMT 1.0 | NMT 1.0 |
| AEX pool % full | 71.4% | 74.7% |
| capsid (AUC) | 57.5% | 75.2% |

Productivities observed at each process step are indicated as total vgs recovered in each step divided by volume of the culture that was processed. Full capsid enrichment step had a higher than 100% step yield for Bioreactor 008 using the 0.5% octoxynol-9/0.1% DB condition (due in part to qPCR variability). All chemical percentages were w/v.

Similar to low cell density results, high cell density TAB processing resulted in a ˜4 fold increase in recovered vgs demonstrated by productivities at the affinity chromatography elution step. As shown in Table 6, compatibility of the novel harvest conditions were assessed with an AEX chromatography purification step at the 76 L scale (N=2). A difference between reactor 007 and 008 was a change in the repcap plasmid used for transfection. Once an optimized plasmid was used in bioreactor 008, overall enrichment of percent full capsids at the AEX elution pool step was similar between the CTAB and the octoxynol-9/DB only conditions, with both comprising ˜75% full capsids at the end of purification. Overall, the CTAB condition scaled up very well, demonstrating improved productivity values at affinity and AEX elution pools.

Compatibility and scale-up of the 50 mM MgSO₄/0.25% CTAB/0.5% octoxynol-9/0.2% DB harvest method with the rest of the process (e.g., AEX and UFDF) was further demonstrated at the 250 L scale (N=2). In all cases, performance was as expected with productivities and quality attributes in the same range seen for the 1 L and 10 L cell cultures.

TABLE 7
In-process productivity (vg/L) and quality
attribute data for 250 L cultures.

| | Parameter tested | 250 L - 1 | 250 L - 2 |
|---|---|---|---|
| clarified | vg/L | 1.46E+14 | 7.61E+13 |
| lysate | HCD pg/1E+9 vgs | 1.54E+02 | 1.30E+03 |
| | HCP ng/1E+9 vgs | 1.18E+06 | 2.43E+06 |
| Affinity | vg/L | 1.48E+14 | 8.09E+13 |
| eluate | % full capsids | 48.2% (AUC) | 41.3% (AUC) |
| pool | | 1.22 (SEC | 1.14 (SEC |
| | | A260/A280) | A260/A280) |
| | % HMMS | NMT 1.0 | 1.5 |
| | HCD pg/1E+9 vgs | 1.34E+01 | 1.41E+01 |
| | HCP ng/1E+9 vgs | 1.16E−02 | 2.02E−02 |
| AEX | vg/L | 9.07E+13 | 5.90E+13 |
| eluate | % full capsids | 78.4% (AUC) | 71.7% (AUC) |
| pool | | 1.34 (SEC | 1.31 (SEC |
| | | A260/280) | A260/280) |
| | % HMMS | NMT 1.0 | NMT 1.0 |
| | HCD pg/1E+9 vgs | 9.11E+00 | 1.10E+01 |
| | HCP ng/1E+9 vgs | Below LOQ | Below LOQ |

AUC = analytical ultracentrifugation;
SEC = size exclusion chromatography

Drug substance (DS) product quality as tested for both 250 L runs met acceptance criteria based on the tested residuals level, productivity and quality attributes such as % full capsid and % HMMS.

TABLE 8
Drug substance quality attributes

| DS | | | % full | HCD pg/ | HCP ng/ |
|---|---|---|---|---|---|
| attributes | vg/L | % HMMS | capsid | ml vg | 1E+9 |
| 250 L-1 | 8.13E+13 | NMT 1.0 | 80.4% | 1004450 | NMT 20.0 |
| 250 L-2 | 4.41E+13 | 2.3 | 71.1% | 326800 | NMT20 |

Based on this data, the 0.25% w/v CTAB based process using 50 mM MgSO4, 0.5% w/v octoxynol-9 and 0.2% w/v DB was tested at the 2000 L cell culture scale.

TABLE 9
Analytical data for 2000 L scale culture

| Process step | Parameter tested | 2000 L - 1 | 2000 L - 2 |
|---|---|---|---|
| Clarified | vg/L | 1.75E+14 | 9.84E+13 |
| Lysate | HCD pg/1E+9 | 1075.04 | 331.5 |
| | HCP ng/1E+9 | 1.74E+06 | 2.61E+06 |
| Affinity | vg/L | 1.69E+14 | 8.54E+13 |
| pool | % monomer | 98.5% | 97.1% |
| | % full | 46.6% | 40.2% |
| | [CTAB] ug/mL | 39.1 | NMT 5.0 |
| | HCD pg/1E+9 | 16.25 | 0.005 |
| | HCP ng/1E+9 | 2.52 | NMT 0.47 |
| AEX pool | vg/L | 1.06E+14 | 6.56E+13 |
| | % monomer | 95.3% | 97.7% |
| | % full | 80.2% | 76.7% |
| | [CTAB] ug/mL | NMT 5.0 | NMT 5.0 |
| | HCD pg/1E+9 | 8.78 | 4.89 |
| | HCP ng/1E+9 | 1.1 | NMT 2.2 |

Viral filtration was tested on the 2000 L scale process and shown to have a high step yield of >90% for 2000L-1 process. DS quality attributes (Table 10) were generally unchanged as compared to the AEX pool quality attributes (Table 9).

TABLE 10
Drug substance quality attributes

| DS | | % | % full | Residual CTAB | HCD pg/ | HCP ng/ |
|---|---|---|---|---|---|---|
| attribute | vg/L | monomer | capsid | ug/mL | 1E+9 vg | 1E+9 vg |
| 2000L-1 | 9.19E+13 | 97.8 | 77.4 | NMT 5.0 | 9.78 | Below LOQ |
| 2000L-2 | 5.5E+13* | 98.2 | 75.5% | NMT 5.0 | 3.04 | Below LOQ |

*Estimated. Mass of DS post final filtration was not recorded, pre filtration value was used to arrive at number indicated in a table above. Minimal changes to volume were expected during final filtration step based on process history.

### Example 6: CTAB Operational Range as a Lysis and Flocculation Reagent

Given the results obtained for the MgSO₄ plus CTAB only process (Examples 2 and 4), the CTAB operating concentration range for this combination was determined. In all cases, MgSO₄ was present as an accompanying salt and the concentration was kept constant at 50 mM. The goal was to establish the lowest amount of CTAB that would operate effectively as both a lysis and flocculation reagent, while maintaining product quality and boosting product recovery. It was recognized that if CTAB alone was successful as a lysis and flocculation agent, octoxynol-9 would no longer be needed in the process. This would be beneficial in that certain regulatory regimes have discouraged or eliminated use of octoxynol-9 in manufacturing processes.

A concentration range of 0.01%-0.05% w/v for CTAB and 50 mM MgSO₄ condition was evaluated for productivity. Addition of MgSO₄ and CTAB was done sequentially, with MgSO₄ added first and allowed to incubate for 15 minutes at RT with stirring, followed by addition of CTAB to a desired final concentration using a 10% stock solution. CTAB was allowed to incubate for 30 minutes at RT with stirring. The percentage of CTAB selected for these studies was lower than the percentage (0.25% w/v) used in the combination with MgSO₄, octoxynol-9 and DB. Bioreactor productivities were calculated at the stage of clarified lysate, affinity elution pool and AEX elution pool (when deemed appropriate). The impact on product quality with the lower CTAB harvest conditions was evaluated by determining the % monomer, analytical ultracentrifugation (AUC) or SEC A260/A280 ratio of the affinity elution pool. Both AUC and SEC A260/A280 ratio are a measure of the % of full capsids in the sample (Table 12). There was no affinity eluate pool material available for analysis from run 016 due to a technical issue which occurred during the affinity chromatography step.

TABLE 11
Process productivity for lysis and flocculation using CTAB

| | | Vg/L | Vg/L | % | vg/L | % |
|---|---|---|---|---|---|---|
| Bioreactor | | clarified | affinity | monomer | AEX | monomer |
| ID/Scale | Harvest method | lysate | pool | affinity | pool | AEX |
| 010/1 L | 50 mM MgSO4/0.05% CTAB | 1.9e+13 | 1.1e+14 | 99.3 | 4.4e+13 | >99 |
| | control condition | 1.6e+14 | 2.3e+14 | 97.8 | 2.31e+13 | >99 |
| 012/10 L | 50 mM MgSO4/0.05% CTAB | 9.1e+13 | 8.34e+13 | 99.5 | No AEX was | |
| | control condition | 1.5e+14 | 1.3e+14 | 99 | performed | |
| 014/10 L | 50 mM MgSO4/0.05% CTAB | 1.3e+14 | 5.7e+13 | 99.5 | 7.9e+13 | >99 |
| | 50 mM MgSO4/0.025% CTAB | 1.9e+14 | 7.4e+13 | 99.9 | 7.6e+13 | >99 |
| | control condition | 1.5e+14 | 5.4e+13 | 99.4 | 3.7e+13 | >99 |
| 016/10 L | 50 mM MgSO4/0.05% CTAB | 3.9e+14 | n/a | n/a | No AEX was | |
| | 50 mM MgSO4/0.025% CTAB | 4.2e+14 | 2.6e+14 | 99.2 | performed | |
| | 50 mM MgSO4/0.01% CTAB | 2.8e+14 | n/a | n/a | | |
| | control condition | 2.9e+14 | 1.3e+14 | 98 | | |

Control condition: 50 mM MgSO₄, 0.25% CTAB, 0.5% octoxynol-9 and 0.2% DB (all percentages w/v).

These data demonstrated that lower CTAB concentrations (0.01% to 0.05% w/v) plus 50 mM MgSO₄ did not result in significantly lower vg recoveries as compared to the combination of MgSO₄, CTAB, octoxynol-9 and DB (control condition). CTAB plus MgSO₄ alone also appeared to have a positive effect on AEX chromatography step yield (vg/L), as on average, higher recoveries were observed as compared to the combination of MgSO₄, CTAB, octoxynol-9 and DB. As far as product quality, no difference was observed with respect to capsid aggregation as judged by % monomer in the affinity elution pool and the AEX elution pool, as well as no difference in % full capsid as measured by either AUC or SEC A260/A280 data.

TABLE 12
Evaluation of full capsid content for
lysis and flocculation using CTAB

| | | % full at | SEC 260/280 |
|---|---|---|---|
| | | affinity | at affinity |
| ID | Harvest method | pool (AUC) | pool |
| 012-10L | 50 mM MgSO₄/0.05% CTAB | 56.4 | not |
| | Control condition | 56.6 | tested |
| 016-10L | 50 mM MgSO₄/0.025% CTAB | not | 1.24 |
| | Control condition | tested | 1.29 |

Control condition: 50 mM MgSO₄, 0.25% CTAB, 0.5% octoxynol-9 and 0.2% DB (all percentages w/v).

These data demonstrated that CTAB was useful as a detergent alternative to octoxynol-9, as the data demonstrated that CTAB is capable of effectively lysing cells and recovering the rAAV3B vectors even at a low concentration (0.01% w/v) of CTAB. Furthermore, the quality of rAAV3B vector recovered using 50 mM MgSO₄/0.025% w/v CTAB to prepare the clarified lysate was equivalent to the quality of the rAAV3B vector recovered using the combination of MgSO₄, CTAB, octoxynol-9 and DB.

### Example 7: Operational Concentration Range of CTAB as a Flocculation Reagent

CTAB was reported in the literature to function as a DNA precipitation reagent (Adbel-Latif and Osman, Plant Methods (2017) 13:1; Minas et al., FEMS Microbiol. Lett (2011) 325:162-169). No increase in system pressures were observed during either the affinity chromatography step or the AEX chromatography step when a clarified lysate was prepared using CTAB with MgSO₄, or in combination in MgSO₄, octoxynol-9 and DB, as described in the preceding Examples. Thus, it was hypothesized that CTAB could also function as an effective flocculant of host cell DNA without the need for DB. The ability of CTAB to clear host cell DNA (HCD) from clarified lysate was assessed at three concentrations (0.01%, 0.025% and 0.05%) and compared to HCD clearance by the combination of CTAB, MgSO₄, octoxynol-9 and DB (Table 13).

TABLE 13
Residual HCD levels (total ng DNA/1E+9 vg) in clarified lysate

| | HCD ng/1E+9 vg at clarified lysate | |
|---|---|---|
| Harvest method | Run No. 012 | Run No. 016 |
| 50 mM MgSO₄/0.25% CTAB/ | 6.93E+01 | 1.98E+01 |
| 0.5% octoxynol-9/0.2% DB | | |
| 50 mM MgSO₄/0.05% CTAB | 2.81E+02 | 2.33E+02 |
| 50 mM MgSO₄/0.025% CTAB | not tested | 4.22E+03 |
| 50 mM MgSO₄/0.01% CTAB | not tested | 1.41E+04 |

The ability of CTAB to clear HCD from an affinity eluate pool was assessed at three concentrations (0.01%, 0.025% and 0.05%) and compared to HCD clearance by the combination of CTAB/MgSO₄/octoxynol-9/DB (Table 10).

TABLE 14
Residual HCD levels (total pg DNA/1E+9 vg)
in affinity eluate pool

| | HCD pg/1E+9 at affinity pool | | | | | |
|---|---|---|---|---|---|---|
| Harvest method | TS4 | TS5 | 007 | 008 | 014 | 016 |
| 0.5% octoxynol-9/0.2% DB | NT | NT | 19 | 9 | NT | NT |
| 50 mM MgSO4/0.25% | 34 | 26 | NT | 3 | 12 | 0.4 |
| CTAB/0.5% octoxynol-9/ | | | | | | |
| 0.2% DB | | | | | | |
| 50 mM MgSO₄/0.05% CTAB | NT | NT | NT | NT | 18 | NT |
| 50 mM MgSO₄/0.05% CTAB | NT | NT | NT | NT | 15 | 3 |

NT = not tested

Detected residual HCD levels in a clarified lysate using a CTAB process with concentrations of (0.01%, 0.025% and 0.05%) were within an acceptable range as compared to both a 0.5% octoxynol-9/0.2% DB and a 50 mM MgSO₄/0.25% CTAB/0.5% octoxynol-9/0.2% DB harvest methods. Detected residual HCD levels in an affinity eluate pool using a CTAB process with concentrations of 0.025% and 0.05% were within an acceptable range as compared to both a 0.5% octoxynol-9/0.2% DB and a 50 mM MgSO₄/0.25% CTAB/0.5% octoxynol-9/0.2% DB harvest method. The data demonstrated that harvest method conditions containing low CTAB concentrations (0.01% and 0.025% w/v) could achieve efficient flocculation as well as cell lysis and product recovery, thus providing an advantageous alternative to octoxynol-9/DB processing.

Additional host cell DNA clearance data at the clarified lysate process step was generated for CTAB at different concentrations using high cell density (˜30E+6 cells/mL) cell culture for the production of a rAAV9 vector comprising a Duchenne muscular dystrophy transgene (SEQ ID NO:2). A Picogreen based DNA detection assay was used to the detect amount of HCD present in the clarified lysate. The experiment was conducted per manufacturing protocol, see Invitrogen User Guide “Quant-iT Picogreen dsDNA Reagent and Kit (Pub. No. MAN0001931 Rev A.0). In short Picogreen reagent (200× stock) was added to 15 mL of clarified lysate in an 50 mL Falcone tube to achieve final concentration of picogreen reagent of 1×. The reaction was allowed to proceed for 15 minutes at RT while mixing on a rocker. Upon completion of reaction time fluorescent measurement was taken with excitation wavelength set to 480 nm and emission to 520 nm. Standard curve was generated per manufacturer's protocol and used to process collected raw data. Lysis and flocculation using 50 mM MgSO₄ and 0.25% CTAB exhibited improved HCD clearance as compared to lower amounts of CTAB, though adequate levels of HCD removal (<1E+07) were demonstrated using a CTAB concentration as low as 0.05% w/v (Table 15).

TABLE 15
HCD levels in clarified lysate

| Sample ID | Harvest method | HCD, pg/mL |
|---|---|---|
| HCD 1 | Raw unlysed harvest | 1.59E+07 |
| HCD 2 | 0.5% octoxynol-9 and no DB | 3.06E+07 |
| HCD 3 | 0.5% oxtoxynol-0/0.25% DB | 3.83E+06 |
| HCD 4 | 50 mM MgSO4/0.25% CTAB | 1.68E+06 |
| HCD 5 | 50 mM MgSO4/0.1% CTAB | 5.18E+06 |
| HCD 6 | 50 mM MgSO4/0.05% CTAB | 9.23E+06 |
| HCD 7 | 50 mM MgSO4/0.025% CTAB | 2.63E+07 |
| HCD 8 | 50 mM MgSO4/0.01% CTAB | 2.42E+07 |

Overall, the performance of the 50 mM MgSO₄/0.01%-0.05% w/v CTAB harvest method was equivalent to that of the current process with respect to both recovery and productivity as well as residual HCD clearance. The combination of 50 mM MgSO₄ and 0.025% CTAB as a lysis (detergent) and flocculation reagent is a suitable replacement for use of 0.5% octoxynol-9/0.2% DB to prepare a rAAV3B vector clarified lysate (Table 16).

TABLE 16
Process performance

| | Harvest method | | |
|---|---|---|---|
| | 50 mM MgSO4/ | 50 mM | 50 mM |
| | 0.25% CTAB/ | MgSO₄ | MgSO₄ |
| | 0.5% octoxynol- | 0.05% | 0.025% |
| | 9/0.2% DB | CTAB | CTAB |
| Bioreactor | 1.1E+14 vg/L | 7.94E+13 vg/L | 1.45E+14 vg/L |
| productivity at | | | |
| affinity pool, vg/L | | | |
| % full capsid at | 53% | 56.4% | 62.9% |
| affinity pool | | | |
| HCD pg/1E+9 vg at | 24 | 190 | 29 |
| affinity pool | | | |
| HCP pg/1E+9 vg at | 91 | 530 | 77 |
| affinity pool | | | |
| Bioreactor | 5.5E+13 | 5.1E+13 | 7.6E+13 |
| productivity at AEX | | | |
| pool, vg/L | | | |
| SEC A260/A280 | 1.33 SEC | 1.34 SEC | 1.35 SEC |
| | A260/A280 | A260/A280 | A260/A280 |

### Example 8: Use of CTAB for Lysis and Flocculation of rAAV3B Vector Comprising a Glucosylceramidase Beta (GBA) Transgene

HEK293 cells were grown in 10 L low cell density suspension cultures and transfected with 3 plasmids to produce a rAAV3B vector using standard methods known in the art. The rAAV3B vector contained a vector genome with a transgene encoding a polypeptide comprising the amino acid sequence of SEQ ID NO:3 (glucosylceramidase beta polypeptide). The HEK293 cells were harvested, lysed and flocculated using 50 mM MgSO₄ with 0.1% (w/v) CTAB (reactor 005, Table 17). In this study, a solution comprising MgSO₄ was added to the cell culture followed by a solution comprising CTAB. The lysate was filtered to produce a clarified lysate. A separate cell feed material grown under the same conditions was lysed and flocculated using 0.5% (w/v) octoxynol-9 with 0.3% (w/v) DB. The productivity as measured by vg/L in the affinity elution pool was comparable between harvest methods.

TABLE 17
Productivity at affinity elution pool
for rAAV3B vector with GBA transgene.

| Cell density | LCD | |
|---|---|---|
| Scale | 10 L | |
| Bioreactor no. | 002 | 005 |
| Harvest method | 0.5% octoxynol-9/ | 50 mM MgSO₄/ |
| | 0.3% DB | 0.1% CTAB |
| Productivity at affinity elution | 4.5E+13 vg/L | 7.1E+13 vg/L |
| pool | | |
| SEC A260/A280 affinity pool | 1.23 | 1.29 |
| Affinity resin used | AAVx | AAVx |

### Example: 9: Use of CTAB for Lysis and Flocculation of rAAV9 Vector Comprising a Therapeutic Transgene

HEK293 cells were grown in a 1 L low cell density suspension culture and transfected with 3 plasmids to produce a rAAV9 vector using standard methods known in the art. HEK293 cells were harvested, lysed, flocculated, and the resulting lysate was filtered to produce a clarified lysate. The cells were lysed and flocculated using 50 mM MgSO₄ with 0.05% (w/v) CTAB or using 0.5% (w/v) octoxynol-9 with 0.2% (w/v) DB. The productivity as measured by vg/L in the clarified lysate and affinity elution pool was comparable between harvest methods. Product quality and product recovery at the clarified lysate and affinity elution pool steps were also comparable between the two methods. While the rAAV9 capsid serotype does not have the same strong affinity for heparin receptors as the rAAV3B serotype, this Example demonstrated that the combination of CTAB and MgSO₄ was useful for preparation of a clarified lysate comprising other vectors, including rAAV9 vectors, and eliminated use of less desirable reagents such as octoxynol-9 and DB from gene therapy vector purification processes.

TABLE 18
Productivity at affinity elution pool for rAAV9 vector

| Cell density | LCD | | | |
|---|---|---|---|---|
| Scale | 1 L | | | |
| Bioreactor no. | 004 | | 006 | |
| Harvest method | 0.5% | 50 mM MgSO4 | 0.5% | 50 mM MgSO4 |
| | octoxynol-9 | 0.05% CTAB | octoxynol-9 | 0.05% CTAB |
| | 0.2% DB | | 0.2% DB | |
| Productivity | 5.0E+14 vg/L | 5.3E+14 vg/L | 4.1E+14 vg/L | 3.7E+14 vg/L |
| at clarified lysate | | | | |
| Productivity | 2.6E14 vg/L | 2.4E14 vg/L | 1.9E+14 vg/L | 2.5E+14 vg/L |
| at affinity elution | | | | |
| pool | | | | |
| SEC A260/A280 | 1.16 | 1.12 | 1.19 | 1.19 |
| affinity elution pool | | | | |

| | |
|---|---|
| Affinity resin used | AAV9 |

The use of a MgSO₄/CTAB harvest method was identified as providing better product quality and recovery as comparted to a 0.5% Triton/0.2% DB harvest method in small scale screens. The CTAB conditions were shown to be scalable and improved process performance at the 1 L, 10 L and 250 L cell culture scales, as judged by bioreactor productivity (total vg/L bioreactor) at clarified lysate, affinity pool, AEX pool and drug substance (250 L only). No impact on product quality was detected.

Evaluation of a lower concentration range of CTAB (less than 0.1%) showed that CTAB detergent was capable of effectively lysing and recovering rAAV3B vectors even at a concentration of 0.025% w/v. The ability of CTAB to operate at such low concentrations while maintaining capsid product quality attributes, makes it an excellent candidate for rAAV gene therapy vector harvest methods, and to replace the use of octoxynol-9.

Furthermore, the absence of increased pressure readings across both depth filter and affinity chromatography unit operations and the clearance of HCD from the harvest demonstrated that the combination of MgSO₄ and CTAB could be used for flocculation as well as lysis. This eliminated the need for an additional reagent (e.g., DB) and shortened the process time since the lysis and flocculation occurred simultaneously. The processing time using MgSO₄ and CTAB was also shorter than the processing time when using DB because there was no flocculant mass that required settling prior to filtration. Also without a flocculant mass there is no loss of residual harvest solution within the mass, thereby increasing the amount of harvest solution that enters the filtration step of the purification process.

### Example 10: Use of CTAB for Viral Clearance

The goal of this study was to evaluate viral clearance of a clarified lysate using CTAB and to compare the clearance to viral clearance using octoxynol-9. The clarified lysate comprised a gene therapy vector.

Clarified lysate prepared from a host cell culture expressing an AAV gene therapy vector comprising a dystrophin transgene was used for the viral clearance assessment. Three concentrations of CTAB were studied, 0.25% (w/v), 0.05% (w/v) and 0.025% (w/v) and the results were compared to viral clearance using 0.5% (wv) octoxynol-9. The octoxynol-9 control was used for direct comparison of clearance levels between the two detergents. In total, four separate clarified lysate samples were prepared, each in duplicate.

Host cell culture samples were generated by aliquoting 0.75 L of culture into four PETG bottles. 2 M MgSO₄ stock solution was added to three of the bottles to achieve a final concentration of 50 mM MgSO₄. After 15 minutes of stirring (stir bar at 350 rpm) at room temperature, an aliquot of 10% (w/v) CTAB stock solution was added to each of the three bottles to achieve a final CTAB concentration of 0.25% (w/v), 0.05% (w/v), or 0.025% (w/v).

The octoxynol-9 control was prepared by adding an aliquot of a 10% stock solution to the fourth bottle to achieve a final concentration of 0.5% (w/v). After 30 minutes of stirring at room temperature, an aliquot of a 10% domiphen bromide (DB) stock solution was added to achieve a final concentration of 0.2% (w/v).

Following chemical addition and incubation, all samples were filtered through a depth filter, followed by application of a chase to the filter to ensure complete removal of clarified lysate from the depth filter. This resulted in dilution of the percent CTAB in the clarified lysate from 0.25% (w/v) to 0.2% (w/v), from 0.05% (w/v) to 0.04% (w/v) and from 0.025% (w/v) to 0.02% (w/v). The percent octoxynol-9 in the clarified lysate was reduced from 0.5% (w/v) to 0.4% (w/v).

The conductivity of all test samples was adjusted to −35 mS/cm using 5 M NaCl stock as a titrant. Final absolute filtration was performed prior to freezing of samples before testing in the viral clearance assay (performed by Charles River Laboratories Wayne, PA).

Murine leukemia virus (MuLV) and pseudo rabies virus (PRV) were spiked with detergent containing samples at a spike ratio of 10% (v/v) to start inactivation. Two spiked samples were tested, and they were stored in the water bath at 18+/1° C. during the entire process (240 minutes total). Samples were withdrawn and analyzed per virus titer.

TABLE 19
Process parameters for viral inactivation using
0.25% CTAB and 0.025% CTAB test samples.

| | MuLV | | PRV | |
|---|---|---|---|---|
| Process parameter | Run 1 | Run 2 | Run 1 | Run 2 |
| Vol. of test sample (mL) | 15 | 15 | 15 | 15 |
| Vol. of viral sample (mL) | 1.5 | 1.5 | 1.5 | 1.5 |
| Vol. of inactivation assay (mL) | 16.5 | 16.5 | 16.5 | 16.5 |

TABLE 20
Process parameters for viral inactivation using
0.05% CTAB and 0.5% octoxynol-9 test samples.

| | MuLV | | PRV | |
|---|---|---|---|---|
| Process parameter | Run 1 | Run 2 | Run 1 | Run 2 |
| Vol. of test sample (mL) | 10 | 10 | 10 | 10 |
| Vol. of viral sample (mL) | 1 | 1 | 1 | 1 |
| Vol. of inactivation assay (mL) | 11 | 11 | 11 | 11 |

Log reduction values were obtained by comparing the titer of the control sample (no detergent) to the titer of the test sample. Samples were assay by both Limiting Dilution (Titer) and Bulk Analysis (large volume plating, LVP). The Bulk Analysis testing utilized a larger volume of material giving a higher sensitivity than the Limiting Dilution. The Bulk Analysis could only calculate a viral titer for the sample when less than 50% of the wells were detected. If less than 50% of the wells were detected, the Bulk Analysis value obtained was used in the comparison.

TABLE 21
MuLV results summary.

| | | | Process demonstrated viral |
|---|---|---|---|
| | Minimum reduction values | | removal or inactivation (Log |
| Process | Run 1 | Run 2 | reduction >1) |
| 0.25% CTAB, 90 mins¹ | ≥4.37 ± 0.31 | ≥4.31 ± 0.25 | Demonstrated inactivation |
| 0.025% CTAB, 90 mins² | 0.47 ± 0.36 | 0.23 ± 0.37 | Did not demonstrate inactivation |
| 0.05% CTAB, 90 mins¹ | ≥4.00 ± 0.23 | ≥4.18 ± 0.25 | Demonstrated inactivation |
| 0.5% octoxynol-9, 90 mins¹ | ≥4.43 ± 0.25 | ≥4.49 ± 0.27 | Demonstrated inactivation |

¹value from large volume plating
²value from titer plate

TABLE 22
PRV results summary.

| | | | Process demonstrated viral |
|---|---|---|---|
| | Minimum reduction values | | removal or inactivation (Log |
| Process | Run 1 | Run 2 | reduction >1) |
| 0.25% CTAB, 90 mins¹ | ≥4.73 ± 0.25 | ≥4.73 ± 0.31 | Demonstrated inactivation |
| 0.025% CTAB, 90 mins² | 0.71 ± 0.44 | 0.71 ± 0.39 | Did not demonstrate inactivation |
| 0.05% CTAB, 90 mins¹ | ≥4.31 ± 0.27 | ≥4.67 ± 0.21 | Demonstrated inactivation |
| 0.5% octoxynol-9, 90 mins¹ | ≥4.43 ± 0.28 | ≥4.19 ± 0.18 | Demonstrated inactivation |

¹value from large volume plating
²value from titer plate

Viral clearance levels for the 0.25% w/v and 0.05% w/v CTAB concentration conditions were comparable to octoxynol-9 viral clearance levels. The 0.025% w/v CTAB condition showed minimal to no viral clearance ability. The 0.05% CTAB concentration condition and 0.5% octoxynol-9 condition required the same amount of time to achieve similar viral clearance levels, i.e., 90 minutes.

SEQUENCES

| SEQ ID | |
|---|---|
| NO | DESCRIPTION AND SEQUENCE |
| SEQ ID | copper transporting ATPase 2 polypeptide with deletion of |
| NO: 1 | metal binding sites 1-4 |
| | MPEQERQITAREGASRKILSKLSLPTRAWEPAMKKSFAFDNVGYEGGLDGLGPSSQPQKC |
| | FLQIKGMTCASCVSNIERNLQKEAGVLSVLVALMAGKAEIKYDPEVIQPLEIAQFIQDLG |
| | FEAAVMEDYAGSDGNIELTITGMTCASCVHNIESKLTRINGITYASVALATSKALVKFDP |
| | EIIGPRDIIKIIEEIGFHASLAQRNPNAHHLDHKMEIKQWKKSFLCSLVFGIPVMALMIY |
| | MLIPSNEPHQSMVLDHNIIPGLSILNLIFFILCTFVQLLGGWYFYVQAYKSLRHRSANMD |
| | VLIVLATSIAYVYSLVILVVAVAEKAERSPVTFFDTPPMLFVFIALGRWLEHLAKSKTSE |
| | ALAKLMSLQATEATVVTLGEDNLIIREEQVPMELVQRGDIVKVVPGGKFPVDGKVLEGNT |
| | MADESLITGEAMPVTKKPGSTVIAGSINAHGSVLIKATHVGNDTTLAQIVKLVEEAQMSK |
| | APIQQLADRFSGYFVPFIIIMSTLTLVVWIVIGFIDFGVVQRYFPNPNKHISQTEVIIRF |
| | AFQTSITVLCIACPCSLGLATPTAVMVGTGVAAQNGILIKGGKPLEMAHKIKTVMFDKTG |
| | TITHGVPRVMRVLLLGDVATLPLRKVLAVVGTAEASSEHPLGVAVTKYCKEELGTETLGY |
| | CTDFQAVPGCGIGCKVSNVEGILAHSERPLSAPASHLNEAGSLPAEKDAVPQTFSVLIGN |
| | REWLRRNGLTISSDVSDAMTDHEMKGQTAILVAIDGVLCGMIAIADAVKQEAALAVHTLQ |
| | SMGVDVVLITGDNRKTARAIATQVGINKVFAEVLPSHKVAKVQELONKGKKVAMVGDGVN |
| | DSPALAQADMGVAIGTGTDVAIEAADVVLIRNDLLDVVASIHLSKRTVRRIRINLVLALI |
| | YNLVGIPIAAGVFMPIGIVLQPWMGSAAMAASSVSVVLSSLQLKCYKKPDLERYEAQAHG |
| | HMKPLTASQVSVHIGMDDRWRDSPRATPWDQVSYVSQVSLSSLTSDKPSRHSAAADDDGD |
| | KWSLLLNGRDEEQYI |
| SEQ ID | Dystrophin3978 polypeptide |
| NO: 2 | MLWWEEVEDCYEREDVQKKTFTKWVNAQFSKFGKQHIENLFSDLQDGRRLLDLLEGLTGQ |
| | KLPKEKGSTRVHALNNVNKALRVLQNNNVDLVNIGSTDIVDGNHKLTLGLIWNIILHWQV |
| | KNVMKNIMAGLQQTNSEKILLSWVRQSTRNYPQVNVINFTTSWSDGLALNALIHSHRPDL |
| | FDWNSVVCQQSATORLEHAFNIARYQLGIEKLLDPEDVDTTYPDKKSILMYITSLFQVLP |
| | QQVSIEAIQEVEMLPRPPKVTKEEHFQLHHOMHYSQQITVSLAQGYERTSSPKPRFKSYA |
| | YTQAAYVTTSDPTRSPFPSQHLEAPEDKSFGSSLMESEVNLDRYQTALEEVLSWLLSAED |
| | TLQAQGEISNDVEVVKDQFHTHEGYMMDLTAHQGRVGNILQLGSKLIGTGKLSEDEETEV |
| | QEQMNLLNSRWECLRVASMEKQSNLHRVLMDLQNQKLKELNDWLTKTEERTRKMEEEPLG |
| | PDLEDLKRQVQQHKVLOEDLEQEQVRVNSLTHMVVVVDESSGDHATAALEEQLKVLGDRW |
| | ANICRWTEDRWVLLQDQPDLAPGLTTIGASPTQTVTLVTQPVVTKETAISKLEMPSSLML |
| | EVPTHRLLQQFPLDLEKFLAWLTEAETTANVLQDATRKERLLEDSKGVKELMKQWQDLQG |
| | EIEAHTDVYHNLDENSQKILRSLEGSDDAVLLQRRLDNMNFKWSELRKKSLNIRSHLEAS |
| | SDQWKRLHLSLQELLVWLQLKDDELSRQAPIGGDFPAVQKQNDVHRAFKRELKTKEPVIM |
| | STLETVRIFLTEQPLEGLEKLYQEPRELPPEERAQNVTRLLRKQAEEVNTEWEKLNLHSA |
| | DWQRKIDETLERLQELQEATDELDLKLRQAEVIKGSWQPVGDLLIDSLQDHLEKVKALRG |
| | EIAPLKENVSHVNDLARQLTTLGIQLSPYNLSTLEDLNTRWKLLQVAVEDRVRQLHEAHR |
| | DFGPASQHFLSTSVQGPWERAISPNKVPYYINHETQTTCWDHPKMTELYQSLADLNNVRF |
| | SAYRTAMKLRRLQKALCLDLLSLSAACDALDQHNLKQNDQPMDILQIINCLTTIYDRLEQ |
| | EHNNLVNVPLCVDMCLNWLLNVYDTGRTGRIRVLSFKTGIISLCKAHLEDKYRYLFKQVA |
| | SSTGFCDQRRLGLLLHDSIQIPRQLGEVASFGGSNIEPSVRSCFQFANNKPEIEAALFLD |
| | WMRLEPQSMVWLPVLHRVAAAETAKHQAKCNICKECPIIGFRYRSLKHFNYDICQSCFFS |
| | GRVAKGHKMHYPMVEYCTPTTSGEDVRDFAKVLKNKFRTKRYFAKHPRMGYLPVQTVLEG |
| | DNMET |
| SEQ ID | glucosylceramidase beta polypeptide |
| NO: 3 | ARPCIPKSFGYSSVVCVCNATYCDSFDPPTFPALGTFSRYESTRSGRRMELSMGPIQANH |
| | TGTGLLLTLQPEQKFQKVKGFGGAMTDAAALNILALSPPAQNLLLKSYFSEEGIGYNIIR |
| | VPMASCDFSIRTYTYADTPDDFQLHNFSLPEEDTKLKIPLIHRALQLAQRPVSLLASPWT |
| | SPTWLKTNGAVNGKGSLKGQPGDIYHQTWARYFVKFLDAYAEHKLQFWAVTAENEPSAGL |
| | LSGYPFQCLGFTPEHQRDFIARDLGPTLANSTHHNVRLLMLDDQRLLLPHWAKVVLTDPE |
| | AAKYVHGIAVHWYLDFLAPAKATLGETHRLFPNTMLFASEACVGSKFWEQSVRLGSWDRG |
| | MQYSHSIITNLLYHVVGWTDWNLALNPEGGPNWVRNFVDSPIIVDITKDTFYKQPMFYHL |
| | GHFSKFIPEGSQRVGLVASQKNDLDAVALMHPDGSAVVVVLNRSSKDVPLTIKDPAVGFL |
| | ETISPGYSIHTYLWRRQ |

1. A method of preparing a cell lysate, the method comprising lysing host cells and precipitating DNA released from the host cells to produce a lysate comprising a flocculant and a supernatant,
wherein the host cells are suspended in a physiologically compatible fluid, forming a cell suspension, and are lysed by adding to the cell suspension a solution comprising a detergent in a concentration sufficient to cause cell lysis; and
wherein the concentration of detergent is sufficient to precipitate host cell DNA from the lysate.

2.-3. (canceled)

4. The method of claim 1, wherein the detergent is a long chain quaternary ammonium salt.

5. The method of claim 4, wherein the long chain quaternary ammonium salt is cetyltrimethylammonium bromide (CTAB).

6. The method of claim 1, further comprising adding a solution comprising a modifier to the fluid.

7. The method of claim 6, wherein the solution comprising the modifier is added to the fluid before the solution comprising the detergent is added to the fluid.

8. The method of claim 6, wherein the modifier is a salt selected from the group consisting of ZnSO₄, MgSO₄, MgCl₂, Na₂SO₄, NaCl, sodium citrate, sodium acetate, ammonium acetate and a combination thereof.

9.-11. (canceled)

12. The method of claim 6, wherein the cell suspension is mixed with the solution comprising the modifier for at least 5 minutes followed by addition of the solution comprising the detergent followed by mixing for at least 5 minutes.

13. (canceled)

14. The method of claim 1, wherein prior to lysis, the viable cell density of the host cells suspended in the physiologically compatible fluid is at least about 10×10⁶ viable cells (vc)/mL.

15. (canceled)

16. The method of claim 1, wherein the host cells are mammalian cells or insect cells.

17. (canceled)

18. The method claim 1, wherein a final concentration of detergent in the lysate is at least 0.01% (w/v), and optionally about 0.05% (w/v).

19.-20. (canceled)

21. The method of claim 5, wherein prior to lysis, the viable cell density of the host cells in the physiologically compatible fluid is at least about 10×10⁶ vc/mL, and a final concentration of CTAB in the lysate is at least about 0.05% (w/v).

22. The method of claim 6, wherein a final concentration of modifier in the lysate is at least 10 mM, and optionally about 50 mM.

23.-24. (canceled)

25. The method of claim 8, wherein prior to lysis, the viable cell density of the host cells in the physiologically compatible fluid is at least about 10×10⁶ vc/mL, and a final concentration of MgSO₄ in the lysate is at least about 50 mM.

26.-28. (canceled)

29. The method of claim 1, wherein the supernatant is separated from the flocculant by filtration to produce a clarified lysate.

30. The method of claim 8, wherein prior to lysis, the viable cell density of the host cells in the physiologically compatible fluid is at least about 10×10⁶ vc/mL, the final concentration of CTAB in the lysate is at least about 0.01% (w/v) and the final concentration of MgSO₄ in the lysate is at least about 10 mM.

31.-36. (canceled)

37. The method of claim 29, wherein the clarified lysate comprises about 1.5 fold to 10 fold more, 1.5 fold to 7 fold more, or 1.5 fold to 5 fold more vector genomes (vg) as compared to a clarified lysate prepared using octoxynol-9 and domiphen bromide to lyse the host cells and precipitate the host cell DNA.

38.-39. (canceled)

40. The method of claim 29, further comprising purifying a biological product from the clarified lysate by performing a downstream purification processing step.

41. The method of claim 40, wherein the biological product is a recombinant viral vector for expressing a heterologous gene and wherein the recombinant viral vector is an AAV vector comprising a capsid that binds more strongly to HSPG as compared to sialic acid or galactose.

42.-44. (canceled)

45. The method of claim 41, wherein the AAV vector comprises an AAV2, AAV3 (including AAV3A or AAV3B), AAV6, AAV13 or AAV-DJ capsid.

46.-51. (canceled)

52. A biological product produced by the method of claim 1.

53.-57. (canceled)
